SEQUENCE LISTING (25 sequences)

SEQ ID NO: 1 - protein, 6 aa, Artificial Sequence
  Description of Artificial Sequencecleavable ubiquitin fusion polypeptide second branch, ubiquitin moiety, minimal length ubiquitin C-terminal peptide
LRLRGG

SEQ ID NO: 2 - protein, 9 aa, Artificial Sequence
  Description of Artificial Sequencecleavable ubiquitin fusion polypeptide first branch
KSSTYKTVA

SEQ ID NO: 3 - protein, 16 aa, Artificial Sequence
  Description of Artificial Sequencepeptide product of proteolysis
AVSEGTKAVTKYTSSK

SEQ ID NO: 4 - protein, 16 aa, Artificial Sequence
  Description of Artificial Sequencesequence from human histone H2b substrate protein
AVSEGTKAVTXYTSSK

SEQ ID NO: 5 - protein, 11 aa, Artificial Sequence
  Description of Artificial Sequencepeptide product of proteolysis
TLHLVLRLRGG

SEQ ID NO: 6 - protein, 11 aa, Artificial Sequence
  Description of Artificial Sequencepeptide branch from C-terminus of ubiquitin
TLHLVLRLRGX

SEQ ID NO: 7 - protein, 16 aa, Artificial Sequence
  Description of Artificial Sequencemutated sequence from human histone H2b substrate protein
AVSEGTKAVXXYTSSK

SEQ ID NO: 8 - protein, 16 aa, Artificial Sequence
  Description of Artificial Sequencepeptide product of proteolysis
AVSEGXKAVTKYTSSK

SEQ ID NO: 9 - DNA, 2903 bp, Homo sapiens
  deubiquitinating agent UbpM (USP16) cDNA
ctcaattcgtcaccaggaggaagacggagctggctgcccagcccaaaggcccatgaggggatgcagttatgggctctgtcgccgtggattgttattttgtgtcagtaagtaatccataaagtgccaacatgggaaagaaacggacaaagggaaaaactgttccaatcgatgattcctctgaaactttagaacctgtgtgcagacacattagaaaaggattggaacaaggtaatttgaaaaaggctttagtgaatgtggaatggaatatctgccaagactgtaagactgacaataaagtgaaagataaagctgaagaagaaacagaagaaaagccttcagtttggctgtgtcttaaatgtggccatcagggctgtggcagaaattctcaggagcagcatgccttgaagcactatctgacgccaagatctgaacctcactgtctggttcttagtttggacaactggagtgtatggtgttacgtatgtgataatgaggtccagtattgtagttcaaaccagttgggtcaagtggttgattatgtcagaaaacaagccagcattacaactccaaagccagcagagaaagataatggaaatattgaacttgaaaataaaaaattagaaaaagagagtaagaatgaacaagagagagaaaagaaggaaaacatggctaaagagaatcctcccatgaattctccttgccaaataaccgtgaaaggactcagtaatttgggaaacacatgtttcttcaatgcagttatgcagaacttgtcacaaacaccagtgcttagagaactactaaaagaagtgaaaatgtctggaacaattgtaaaaattgaaccacctgatttggcattaacagaaccattagaaataaaccttgagcctccaggccctcttactttagccatgagccagtttcttaatgagatgcaagagaccaaaaagggggttgtgacaccgaaagaactcttttctcaggtctgtaaaaaagcagtgcggtttaaaggctatcagcagcaagacagccaggagctgcttcgctacttattggatgggatgagagcagaagaacaccaaagagtgagtaaaggaatacttaaagcatttggtaattctactgaaaagttggatgaagaactaaaaaataaagttaaagattatgagaagaaaaaatcaatgccaagttttgttgaccgcatctttggtggtgaactaactagtatgatcatgtgtgatcaatgcagaactgtctccttggttcatgaatctttccttgatttgtccctcccagttttagatgatcagagtggtaagaaaagtgtaaatgataaaaatctgaaaaagacagtggaggatgaagatcaagatagtgaggaagaaaaagataacgacagttacataaaagagagaagtgatattccttctggaacaagtaagcacttacagaaaaaagcaaagaaacaagccaaaaagcaagccaagaaccaacgaagacaacaaaaaattcaaggaaaagttcttcatttaaatgatatttgtactattgaccatcctgaagacagtgaatatgaagctgaaatgtcacttcaaggagaagtaaatattaaatccaaccatatttcacaagagggtgttatgcataaagaatattgtgtcaaccagaaagatttgaatggccaagcaaaaatgatcgaaagtgtaactgacaatcaaaaatccacagaggaagtagatatgaaaaatatcaacatggataatgatctggaggttttaacatcttctcccactaggaatttaaatggtgcctacctaacggaagggagcaatggagaagtggacatttccaatggtttcaaaaacctaaatttgaatgctgctcttcatcctgatgaaataaatatagagattctgaatgatagtcatactcctggaacaaaggtgtatgaggttgtaaatgaagatccagaaactgctttctgtactcttgcaaacagggaagttttcaatactgatgagtgttcaatccaacattgtttatatcagttcacccgtaatgagaaacttcgagatgcgaataaactgctttgtgaagtatgcacacggagacagtgtaatggaccaaaggcaaatataaaaggtgaaaggaagcatgtttacaccaatgccaaaaagcagatgctaatttctcttgctcctcctgttcttactcttcatttaaagagatttcagcaggctggttttaacctacgcaaagttaacaaacacataaagtttccggaaatcttagatttggctcctttttgcacccttaaatgtaagaatgttgcagaagaaaatacaagggtactctattccttatatggagttgttgaacacagtggtactatgaggtcggggcattacactgcctatgccaaggcaagaaccgcaaatagtcatctctctaatcttgttcttcacggtgatattccacaagattttgaaatggaatcaaaagggcagtggtttcacatcagcgacacacatgtgcaagctgtgcctacaactaaagtactaaactcacaagcgtacctcctattttatgagagaatactgtaataatatcaaaagcactttttctggaaacacatttatggcttttataatggctgaaataacgataaaaaaagactaattaaaatcatgttcacttaacattaaatacatgccagaagaaatcatgtttatttaaatattgaagggaaaaatacctaaaaatgtacaaaggttttatattgtcatagtggtttttattcctgctttgtttctggaaaggaaatcctgaattacttaagtactttgtgtttaatatatctgggtgatggatcacaacacatcaataaactgacttaccctaaaatc

SEQ ID NO: 10 - protein, 823 aa, Homo sapiens
  deubiquitinating agent UbpM (USP16)
MGKKRTKGKTVPIDDSSETLEPVCRHIRKGLEQGNLKKALVNVEWNICQDCKTDNKVKDKAEEETEEKPSVWLCLKCGHQGCGRNSQEQHALKHYLTPRSEPHCLVLSLDNWSVWCYVCDNEVQYCSSNQLGQVVDYVRKQASITTPKPAEKDNGNIELENKKLEKESKNEQEREKKENMAKENPPMNSPCQITVKGLSNLGNTCFFNAVMQNLSQTPVLRELLKEVKMSGTIVKIEPPDLALTEPLEINLEPPGPLTLAMSQFLNEMQETKKGVVTPKELFSQVCKKAVRFKGYQQQDSQELLRYLLDGMRAEEHQRVSKGILKAFGNSTEKLDEELKNKVKDYEKKKSMPSFVDRIFGGELTSMIMCDQCRTVSLVHESFLDLSLPVLDDQSGKKSVNDKNLKKTVEDEDQDSEEEKDNDSYIKERSDIPSGTSKHLQKKAKKQAKKQAKNQRRQQKIQGKVLHLNDICTIDHPEDSEYEAEMSLQGEVNIKSNHISQEGVMHKEYCVNQKDLNGQAKMIESVTDNQKSTEEVDMKNINMDNDLEVLTSSPTRNLNGAYLTEGSNGEVDISNGFKNLNLNAALHPDEINIEILNDSHTPGTKVYEVVNEDPETAFCTLANREVFNTDECSIQHCLYQFTRNEKLRDANKLLCEVCTRRQCNGPKANIKGERKHVYTNAKKQMLISLAPPVLTLHLKRFQQAGFNLRKVNKHIKFPEILDLAPFCTLKCKNVAEENTRVLYSLYGVVEHSGTMRSGHYTAYAKARTANSHLSNLVLHGDIPQDFEMESKGQWFHISDTHVQAVPTTKVLNSQAYLLFYERIL

SEQ ID NO: 11 - DNA, 5213 bp, Homo sapiens
  deubiquitinating agent USP25 cDNA
aagcagcccgcggaccggcagcaaaggaacgtgcgaacgcgtgacgccgcccgactggctcgcgctctcccgtgccccggcgtcctccgcccgctcatggcccgggccgccgcggacgatcggcgctgaggcgggccgcgtggagacgtgaggcggccgccgtggccctcacagtcggcgtttcgccgcctgcccgcggtgcccgcgcacgcctgccgccatcgccttcgcgcctggctggcgggggcgctgtcctcccaggccgtccgcgccgctccctggagctcggcggagcgcggcagccagggccggcggaggcgcgaggagccgggcgccaccgccgccgccgccgccgccgccgcgggggccatgaccgtggagcagaacgtgctgcagcagagcgcggcgcagaagcaccagcagacgtttttgaatcaactgagagaaattacggggattaatgacacccagatactacagcaagccttgaaggatagtaatggaaacttggaattagcagtggctttccttactgcgaagaatgctaagacccctcagcaggaggagacaacttactaccaaacagcacttcctggcaatgatagatacatcagtgtgggaagccaagcagatacaaatgtgattgatctcactggagatgataaagatgatcttcagagagcaattgccttgagtttggccgaatcaaacagggcattcagggagactggaataactgatgaggaacaagccattagcagagttcttgaagccagcatagcagagaataaagcatgtttgaagaggacacctacagaagtttggagggattctcgaaacccttatgatagaaaaagacaggacaaagctcccgttgggctaaagaatgttggcaatacttgttggtttagtgctgttattcagtcattatttaatcttttggaatttagaagattagttctgaattacaagcctccatcaaatgctcaagatttaccccgaaaccaaaaggaacatcggaatttgccttttatgcgtgagctgaggtatctatttgcacttcttgttggtaccaaaaggaagtatgttgatccatcaagagcagttgaaattcttaaggatgctttcaaatcaaatgactcacagcagcaagatgtgagtgagtttacacacaaattattagattggttagaagatgccttccaaatgaaagctgaagaggagacggatgaagagaagccaaagaaccccatggtagagttgttctatggcagattcctggctgtgggagtacttgaaggtaaaaaatttgaaaacactgaaatgtttggtcagtacccacttcaggtcaatgggttcaaagatctgcatgagtgcctagaagctgcaatgattgaaggagaaattgagtctttacattcagagaattcaggaaaatcaggccaagagcattggtttactgaattaccacctgtgttaacatttgaattgtcaagatttgaatttaatcaggcattgggaagaccagaaaaaattcacaacaaattagaatttccccaagttttatatttggacagatacatgcacagaaacagagaaataacaagaattaagagggaagagatcaagagactgaaagattacctcacggtattacaacaaaggctagaaagatatttaagctatggttccggtcccaaacgattccccttggtagatgttcttcagtatgcattggaatttgcctcaagtaaacctgtttgcacttctcctgttgacgatattgacgctagttccccacctagtggttccataccatcacagacattaccaagcacaacagaacaacagggagccctatcttcagaactgccaagcacatcaccttcatcagttgctgccatttcatcgagatcagtaatacacaaaccatttactcagtcccggatacctccagatttgcccatgcatccggcaccaaggcacataacggaggaaaaactttctgtgctggaaagttgtttacatcgctggaggacagaaatagaaaatgacaccagagatttgcaggaaagcatatccagaatccatcgaacaattgaattaatgtactctgacaaatctatgatacaagttccttatcgattacatgccgttttagttcacgaaggccaagctaatgctgggcactactgggcatatatttttgatcatcgtgaaagcagatggatgaagtacaatgatattgctgtgacaaaatcatcatgggaagagctagtgagggactcttttggtggttatagaaatgccagtgcatactgtttaatgtacataaatgataaggcacagttcctaatacaagaggagtttaataaagaaactgggcagccccttgttggtatagaaacattaccaccggatttgagagattttgttgaggaagacaaccaacgatttgaaaaagaactagaagaatgggatgcacaacttgcccagaaagctttgcaggaaaagcttttagcgtctcagaaattgagagagtcagagacttctgtgacaacagcacaagcagcaggagacccagaatatctagagcagccatcaagaagtgatttctcaaagcacttgaaagaagaaactattcaaataattaccaaggcatcacatgagcatgaagataaaagtcctgaaacagttttgcagtcgatcatgatgacaccgaacatgcaaggtattatcatggcgataggtaaatccaggagtgtatatgacaggtgtggccctgaagcagggttctttaaggcaattaagttggaatatgcaaggttggttaagttggcccaagaagacaccccaccagaaaccgattatcgtttacatcatgtagtggtctactttatccagaaccaggcaccaaagaaaattattgagaaaacattactagaacaatttggagatagaaatttgagttttgatgaaaggtgtcacaacataatgaaagttgctcaagccaaactggaaatgataaaacctgaagaagtaaacttggaggaatatgaggagtggcatcaggattataggaaattcagggaaacaactatgtatctcataattgggctagaaaattttcaaagagaaagttatatagattccttgctgttcctcatctgtgcttatcagaataacaaagaactcttgtctaaaggcttatacagaggacatgatgaagaattgatatcacattatagaagagaatgtttgctaaaattaaatgagcaagccgcagaactcttcgaatctggagaggatcgagaagtaaacaatggtttgattatcatgaatgagtttattgtcccatttttgccattattactggtggatgaaatggaagaaaaggatatactagctgtagaagatatgagaaatcgatggtgttcctaccttggtcaagaaatggaaccacacctccaagaaaagctgacagattttttgccaaaactgcttgattgttctatggagattaaaagtttccatgagccaccgaagttaccttcatattccacgcatgaactctgtgagcgatttgcccgaatcatgttgtccctcagtcgaactcctgctgatggaagataaactgcacactttccctgaacacactgtataaactctttttagttcttaacccttgccttcctgtcacagggtttgcttgttgctgctatagtttttaacttttttttattttaataactgcaaaagacaaaatgactatacagactttagtcagactgcagacaataaagctgaaaatcgcatggcgctcagacattttaaccggaactgatgtataatcacaaatctaattgattttattatggccaaactatgcttttgccaccttcctgttgcagtattactttgcttttatcttttctttctcaacagctttccattcagtctggatccttccatgactacagccatttaagtgttcagcactgtgtacgatacataatatttggtagcttgtaaatgaaataaagaataaagttttatttatggctacctatgtgtttgtaagcaggtatattgtatattagtgtattagtaatactagataaatgaattttgtctggggattaagattggatagttaatagattaatacaatcttttaattctgctctaatgctagcaaattggaaaatgtttaagtctttgacacttaaatttatctatatttttaacaaagttcttgaacttagtatggcaccggaacctgttttgaattcagtcaggtttttactcaagtaagtggtgttttttttaagtcaaactacactgaaacttttatccttttcttagattaatcttactttttaaatgtatttacaatatacagcaaggtgattatttcaagagaatcccaaagtacttgaataagggctattgtaaaatttaaaagaaatatttatatatacacatatatacacatacacacatgtatatatatattcttcataatggaggacaatgttttgcaatatataaatcattctatttttgtaaattgtatatcactttaattgaaaatgttctctactaattaatactgtgaaacaaaattgatgttgtttaactagaagttatgagtatcttaactgcctttattccttttcaaaaagaaaaagctgtagaacattttgtagatgaaactactgtttaagattaatgaattaatattgtgaatgaaaatcaaaatccatactttaaaggtaatcatgttactaaccacctatttttgaattcataaaaatttctttataaatgatgttttgtgaacatagtaaaatagaccattatactatgtgtatgtttgatacagcgtcgccaaaactagtgttctttattagtgcctctcacaaaagatcctggatggaggagtaagatgaaatattatgctattatatgatgctgtttgtaaaggtattaatgtactagtaaggtgttaatgacaaggaattagtactattcctgttgtaaagttagattttgcatattgtatctatcaaaatatgtttgggtttagattttaagttgtctactgagcagatttctgcattggttttccagtcctgttaaaagtttagaaacttcatatgtgtcatcacagcttttgtaaagaaagtatccttaatattttatgacattct

SEQ ID NO: 12 - protein, 1087 aa, Homo sapiens
  deubiquitinating agent USP25
MTVEQNVLQQSAAQKHQQTFLNQLREITGINDTQILQQALKDSNGNLELAVAFLTAKNAKTPQQEETTYYQTALPGNDRYISVGSQADTNVIDLTGDDKDDLQRAIALSLAESNRAFRETGITDEEQAISRVLEASIAENKACLKRTPTEVWRDSRNPYDRKRQDKAPVGLKNVGNTCWFSAVIQSLFNLLEFRRLVLNYKPPSNAQDLPRNQKEHRNLPFMRELRYLFALLVGTKRKYVDPSRAVEILKDAFKSNDSQQQDVSEFTHKLLDWLEDAFQMKAEEETDEEKPKNPMVELFYGRFLAVGVLEGKKFENTEMFGQYPLQVNGFKDLHECLEAAMIEGEIESLHSENSGKSGQEHWFTELPPVLTFELSRFEFNQALGRPEKIHNKLEFPQVLYLDRYMHRNREITRIKREEIKRLKDYLTVLQQRLERYLSYGSGPKRFPLVDVLQYALEFASSKPVCTSPVDDIDASSPPSGSIPSQTLPSTTEQQGALSSELPSTSPSSVAAISSRSVIHKPFTQSRIPPDLPMHPAPRHITEEKLSVLESCLHRWRTEIENDTRDLQESISRIHRTIELMYSDKSMIQVPYRLHAVLVHEGQANAGHYWAYIFDHRESRWMKYNDIAVTKSSWEELVRDSFGGYRNASAYCLMYINDKAQFLIQEEFNKETGQPLVGIETLPPDLRDFVEEDNQRFEKELEEWDAQLAQKALQEKLLASQKLRESETSVTTAQAAGDPEYLEQPSRSDFSKHLKEETIQIITKASHEHEDKSPETVLQSIMMTPNMQGIIMAIGKSRSVYDRCGPEAGFFKAIKLEYARLVKLAQEDTPPETDYRLHHVVVYFIQNQAPKKIIEKTLLEQFGDRNLSFDERCHNIMKVAQAKLEMIKPEEVNLEEYEEWHQDYRKFRETTMYLIIGLENFQRESYIDSLLFLICAYQNNKELLSKGLYRGHDEELISHYRRECLLKLNEQAAELFESGEDREVNNGLIIMNEFIVPFLPLLLVDEMEEKDILAVEDMRNRWCSYLGQEMEPHLQEKLTDFLPKLLDCSMEIKSFHEPPKLPSYSTHELCERFARIMLSLSRTPADGR

SEQ ID NO: 13 - DNA, 1119 bp, Homo sapiens
  deubiquitinating agent Yuh1 homolog ubiquitin carboxyl-terminal esterase L1 (UCHL1) cDNA
ggcacgaggcctgggcggctccgctagctgtttttcgtcttccctaggctatttctgccgggcgctccgcgaagatgcagctcaagccgatggagatcaaccccgagatgctgaacaaagtgctgtcccggctgggggtcgccggccagtggcgcttcgtggacgtgctggggctggaagaggagtctctgggctcggtgccagcgcctgcctgcgcgctgctgctgctgtttcccctcacggcccagcatgagaacttcaggaaaaagcagattgaagagctgaagggacaagaagttagtcctaaagtgtacttcatgaagcagaccattgggaattcctgtggcacaatcggacttattcacgcagtggccaataatcaagacaaactgggatttgaggatggatcagttctgaaacagtttctttctgaaacagagaaaatgtcccctgaagacagagcaaaatgctttgaaaagaatgaggccatacaggcagcccatgatgccgtggcacaggaaggccaatgtcgggtagatgacaaggtgaatttccattttattctgtttaacaacgtggatggccacctctatgaacttgatggacgaatgccttttccggtgaaccatggcgccagttcagaggacaccctgctgaaggacgctgccaaggtctgcagagaattcaccgagcgtgagcaaggagaagtccgcttctctgccgtggctctctgcaaggcagcctaatgctctgtgggagggactttgctgatttcccctcttcccttcaacatgaaaatatatacccccccatgcagtctaaaatgcttcagtacttgtgaaacacagctgttcttctgttctgcagacacgccttcccctcagccacacccaggcacttaagcacaagcagagtgcacagctgtccactgggccattgtggtgtgagcttcagatggtgaagcattctccccagtgtatgtcttgtatccgatatctaacgctttaaatggctactttggtttctgtctgtaagttaagaccttggatgtggtttaattgtttgtcctcaaaaggaataaaacttttctgctgataagataaaaaaaaaaaaaaaaaa

SEQ ID NO: 14 - protein, 223 aa, Homo sapiens [sequence omitted]

SEQ ID NO: 15 - DNA, 3064 bp, Homo sapiens
  deubiquitinating agent Unph proto-oncogene (USP4) cDNA
gagatggcggaaggtggaggctgccgtgagcgaccggatgcggagactcagaagtccgagcttggacccttaatgaggaccacactccaacgcggggcgcagtggtatcttattgacagccggtggttcaagcagtggaagaagtatgtgggctttgacagctgggacatgtacaatgtgggtgaacataacctatttcctggcccaatagacaactctgggctattttcagatcctgagagtcagaccttgaaagaacacttaattgatgaattggactatgtattggtccctaccgaggcgtggaataaactactaaactggtacggctgtgtagaaggccagcaacccatcgtcagaaaagttgtggagcatggcctgtttgtcaagcactgcaaagtcgaggtgtatttgctggaactgaagctctgtgagaacagtgaccccaccaatgtgctgagttgccatttcagcaaggcagacaccattgcaaccatcgagaaagagatgcggaagctattcaacatccctgcggagcgtgaaacacggctctggaacaaatacatgagcaacacctacgagcagttgagcaagctagacaacactgtccaggatgctgggctataccagggtcaggtgctagtaattgagcctcaaaatgaagatggcacatggcccaggcagaccttgcagtcaaaatcaagcactgcgcctagcagaaattttactacctctccaaaatcatcagcaagtccctattcctcagtgtctgcctctctcattgcaaatggtgatagcactagcacctgtgggatgcacagttccggtgtcagcaggggtggatctggcttttctgcttcgtataattgtcaggagccaccatcctctcatatacaacctgggctctgtggacttggaaacctgggaaacacctgcttcatgaactccgctttgcagtgtttgagcaacactgcaccactgactgactactttctcaaagatgagtatgaagccgaaatcaacagagacaaccctctggggatgaaaggggaaattgcagaagcctatgctgaactcattaagcagatgtggtctggaagggacgcccatgtggcaccctcgatgttcaaaactcaagtaggacgttttgctcctcaattttctggctaccagcaacaagattctcaggagctgctggcctttcttctagatggattgcatgaagatctgaaccgggtaaagaaaaagccctacttggagctgaaggatgccaatgggcggccagatgcggtggtggcaaaggaagcctgggagaatcacaggttgaggaatgattctgtgattgtggatactttccatggcctcttcaaatctactttggtttgcccagaatgtgctaaggtttctgtgacctttgacccattttgctatctaacgctgccactgcccttgaagaaagatcgagttatggaggttttcctggttcctgctgaccctcactgcagacctactcagtaccgtgtgactgtgccgctgatgggggctgtgtccgacctgtgcgaggctctctccaggctgtctggcattgctgcagaaaatatggtggtcgcagatgtgtataatcaccgattccacaaaattttccaaatggatgaaggtttaaaccacatcatgcctcgggatgacattttcgtgtacgaggtctgcagcacttccgtggatggctcggaatgtgtcacgcttccagtctacttcagggagaggaagtccaggccatcaagcacttcctccgcatcagcgctatatgggcagccactattgctttctgtccccaagcacaagttaacccttgagtctttgtaccaggctgtttgtgatcgtatcagccgctatgtgaaacagcctttacctgatgagtttggcagctcacccttggagccaggggcctgcaatggctccaggaacagctgtgaaggagaagatgaggaagaaatggagcatcaggaagaaggcaaagagcagctttcagaaacagaaggcagtggggaagatgagccaggaaatgaccccagtgagaccacccaaaagaagatcaaaggccagccctgcccaaaaaggctttttaccttcagtcttgtgaactcctatggaacagctgacataaattcacttgcagctgatggaaaactacttaaactcaactctcgatctacactggccatggattgggacagagaaactcggagactttactatgatgagcaagaatctgaggcctacgagaagcatgtgagcatgttgcagcctcagaagaagaagaagaccacagtggccctgagagactgcatcgagctcttcaccaccatggagacccttggggagcatgacccctggtactgtcccaactgtaagaagcatcaacaggccacaaaaaagtttgacctatggtccttgcccaagatcctggtggtccacctcaaacgtttctcctacaacagatactggagggataagctcgacacagtcgtagaattcccaatcagagggctgaacatgtccgagtttgtctgtaacctgtcagcaaggccttatgtgtacgacctcattgccgtgtccaatcattatggagccatgggggttggccactacactgcatatgcgaagaacaaactgaatggtaaatggtattactttgatgatagcaacgtgtccctggcctctgaggatcagatagtgactaaagcagcttatgtgctattttaccaacgtcgagatgatgaattttataagacaccttcacttagcagttctggttcctctgatggagggacacgaccaagcagctctcagcagggctttggggatgatgaggcttgcagcatggacaccaactaatgctgactccacgatcctgccaccctgtagcgccagtgtaatcccccaggagaacatctttgacactctgcagactgctagtgttctgtctaaaaaccagacaaggaaatacccttcttttatgagcagaaggaaacaaaaaaaaaaagaagaccgtttacctagaaag

SEQ ID NO: 16 - protein, 963 aa, Homo sapiens
  deubiquitinating agent Unph proto-oncogene (USP4)
MAEGGGCRERPDAETQKSELGPLMRTTLQRGAQWYLIDSRWFKQWKKYVGFDSWDMYNVGEHNLFPGPIDNSGLFSDPESQTLKEHLIDELDYVLVPTEAWNKLLNWYGCVEGQQPIVRKVVEHGLFVKHCKVEVYLLELKLCENSDPTNVLSCHFSKADTIATIEKEMRKLFNIPAERETRLWNKYMSNTYEQLSKLDNTVQDAGLYQGQVLVIEPQNEDGTWPRQTLQSKSSTAPSRNFTTSPKSSASPYSSVSASLIANGDSTSTCGMHSSGVSRGGSGFSASYNCQEPPSSHIQPGLCGLGNLGNTCFMNSALQCLSNTAPLTDYFLKDEYEAEINRDNPLGMKGEIAEAYAELIKQMWSGRDAHVAPSMFKTQVGRFAPQFSGYQQQDSQELLAFLLDGLHEDLNRVKKKPYLELKDANGRPDAVVAKEAWENHRLRNDSVIVDTFHGLFKSTLVCPECAKVSVTFDPFCYLTLPLPLKKDRVMEVFLVPADPHCRPTQYRVTVPLMGAVSDLCEALSRLSGIAAENMVVADVYNHRFHKIFQMDEGLNHIMPRDDIFVYEVCSTSVDGSECVTLPVYFRERKSRPSSTSSASALYGQPLLLSVPKHKLTLESLYQAVCDRISRYVKQPLPDEFGSSPLEPGACNGSRNSCEGEDEEEMEHQEEGKEQLSETEGSGEDEPGNDPSETTQKKIKGQPCPKRLFTFSLVNSYGTADINSLAADGKLLKLNSRSTLAMDWDRETRRLYYDEQESEAYEKHVSMLQPQKKKKTTVALRDCIELFTTMETLGEHDPWYCPNCKKHQQATKKFDLWSLPKILVVHLKRFSYNRYWRDKLDTVVEFPIRGLNMSEFVCNLSARPYVYDLIAVSNHYGAMGVGHYTAYAKNKLNGKWYYFDDSNVSLASEDQIVTKAAYVLFYQRRDDEFYKTPSLSSSGSSDGGTRPSSSQQGFGDDEACSMDTN

SEQ ID NO: 17 - DNA, 2063 bp, Homo sapiens
  deubiquitinating agent BRAP cDNA
gttcgaggagctgctgctgctgaggcggcggcaactgcattgaggtggtggcggcgctgccggccccggccgctcgctctcggctcgccttccagcctcgcctgagcccgccgggcccgcgccggccagcgcctgccctatgagtgtgtcactggttgttatccgattggagctcgcggaacactcgcctgtccccgccggcttcggcttcagcgccgcggccggggaaatgtctgatgaggagataaaaaagacgacactagcctcagctgtagcctgtttagaaggcaagtcaccaggagagaaagtagcgattatccatcagcatctcggccgtcgagaaatgacagatgtgatcattgagaccatgaagtccaacccagatgaactaaaaactacagtggaagaaaggaagtcttcagaagcctcccccactgcgcaaagaagtaaagatcacagtaaggaatgcataaacgctgccccagattctccgtccaaacagcttccagaccagatttcattcttcagtggaaatccatcagttgaaatagttcatggtattatgcacctatataagacaaataagatgacctccttaaaagaagatgtgcggcgcagtgccatgctgtgtattctcacagtccctgctgcaatgaccagtcatgaccttatgaagtttgttgccccatttaacgacgtaattgaacaaatgaaaattatcagagactctactcccaaccaatatatggtgctgataaagtttcgtgcacaggctgatgcggatagtttttatatgacatgcaatggccgccagttcaactcaatagaagatgacgtttgccagctagtgtatgtggaaagagctgaagtgctcaaatctgaagatggcgccagcctcccagtgatggacctgactgaactccccaagtgcacggtgtgtctggagcgcatggacgagtctgtgaatggcatcctcacaacgttatgtaaccacagcttccacagccagtgtctacagcgctgggacgataccacgtgtcctgtttgccggtactgtcaaacgcccgagccagtagaagaaaataagtgttttgagtgtggtgttcaggaaaatctttggatttgtttaatatgcggccacataggatgtggacggtatgtcagtcgacatgcttataagcactttgaggaaacgcagcacacgtatgccatgcagcttaccaaccatcgagtctgggactatgctggagataactatgttcatcgactggttgcaagtaaaacagatggaaaaatagtacagtatgaatgtgagggggatacttgccaggaagagaaaatagatgccttacagttagagtattcatatttactaacaagccagctggaatctcagcgaatctactgggaaaacaagatagttcggatagagaaggacacagcagaggaaattaacaacatgaagaccaagtttaaagaaacaattgagaagtgtgataatctagagcacaaactaaatgatctcctaaaagaaaagcagtctgtggaaagaaagtgcactcagctaaacacaaaagtggccaaactcaccaacgagctcaaagaggagcaggaaatgaacaagtgtttgcgagccaaccaagtcctcctgcagaacaagctaaaagaggaggagagggtgctgaaggagacctgtgaccaaaaagatctgcagatcaccgagatccaggagcagctgcgtgacgtcatgttctacctggagacacagcagaagatcaaccatctgcctgccgagacccggcaggaaatccaggagggacagatcaacatcgccatggcctcggcctcgagccctgcctcttcggggggcagtgggaagttgccctccaggaagggccgcagcaagaggggcaagtgaccttcagagcaacagacatccctgagactgttctccctgacactgtgagagtgtgctgggaccttcagctaaatgtgagggtgggccctaataagtacaagtgaggacgaaggccggccttcgtggccttagagatggatgaggc

SEQ ID NO: 18 - protein, 592 aa, Homo sapiens
  deubiquitinating agent BRAP
MSVSLVVIRLELAEHSPVPAGFGFSAAAGEMSDEEIKKTTLASAVACLEGKSPGEKVAIIHQHLGRREMTDVIIETMKSNPDELKTTVEERKSSEASPTAQRSKDHSKECINAAPDSPSKQLPDQISFFSGNPSVEIVHGIMHLYKTNKMTSLKEDVRRSAMLCILTVPAAMTSHDLMKFVAPFNDVIEQMKIIRDSTPNQYMVLIKFRAQADADSFYMTCNGRQFNSIEDDVCQLVYVERAEVLKSEDGASLPVMDLTELPKCTVCLERMDESVNGILTTLCNHSFHSQCLQRWDDTTCPVCRYCQTPEPVEENKCFECGVQENLWICLICGHIGCGRYVSRHAYKHFEETQHTYAMQLTNHRVWDYAGDNYVHRLVASKTDGKIVQYECEGDTCQEEKIDALQLEYSYLLTSQLESQRIYWENKIVRIEKDTAEEINNMKTKFKETIEKCDNLEHKLNDLLKEKQSVERKCTQLNTKVAKLTNELKEEQEMNKCLRANQVLLQNKLKEEERVLKETCDQKDLQITEIQEQLRDVMFYLETQQKINHLPAETRQEIQEGQINIAMASASSPASSGGSGKLPSRKGRSKRGK

SEQ ID NO: 19 - DNA, 3599 bp, Homo sapiens
  deubiquitinating agent BAP1 cDNA
gcccgttgtctgtgtgtgggactgaggggccccgggggcggtgggggctcccggtgggggcagcggtggggagggagggcctggacatggcgctgaggggccgccccgcgggaagatgaataagggctggctggagctggagagcgacccaggcctcttcaccctgctcgtggaagatttcggtgtcaagggggtgcaagtggaggagatctacgaccttcagagcaaatgtcagggccctgtatatggatttatcttcctgttcaaatggatcgaagagcgccggtcccggcgaaaggtctctaccttggtggatgatacgtccgtgattgatgatgatattgtgaataacatgttctttgcccaccagctgatacccaactcttgtgcaactcatgccttgctgagcgtgctcctgaactgcagcagcgtggacctgggacccaccctgagtcgcatgaaggacttcaccaagggtttcagccctgagagcaaaggatatgcgattggcaatgccccggagttggccaaggcccataatagccatgccaggcccgagccacgccacctccctgagaagcagaatggccttagtgcagtgcggaccatggaggcgttccactttgtcagctatgtgcctatcacaggccggctctttgagctggatgggctgaaggtctaccccattgaccatgggccctggggggaggacgaggagtggacagacaaggcccggcgggtcatcatggagcgtatcggcctcgccactgcaggggagccctaccacgacatccgcttcaacctgatggcagtggtgcccgaccgcaggatcaagtatgaggccaggctgcatgtgctgaaggtgaaccgtcagacagtactagaggctctgcagcagctgataagagtaacacagccagagctgattcagacccacaagtctcaagagtcacagctgcctgaggagtccaagtcagccagcaacaagtccccgctggtgctggaagcaaacagggcccctgcagcctctgagggcaaccacacagatggtgcagaggaggcggctggttcatgcgcacaagccccatcccacagccctcccaacaaacccaagctagtggtgaagcctccaggcagcagcctcaatggggttcaccccaaccccactcccattgtccagcggctgccggcctttctagacaatcacaattatgccaagtcccccatgcaggaggaagaagacctggcggcaggtgtgggccgcagccgagttccagtccgcccaccccagcagtactcagatgatgaggatgactatgaggatgacgaggaggatgacgtgcagaacaccaactctgcccttaggtataaggggaagggaacagggaagccaggggcattgagcggttctgctgatgggcaactgtcagtgctgcagcccaacaccatcaacgtcttggctgagaagctcaaagagtcccagaaggacctctcaattcctctgtccatcaagactagcagcggggctgggagtccggctgtggcagtgcccacacactcgcagccctcacccacccccagcaatgagagtacagacacggcctctgagatcggcagtgctttcaactcgccactgcgctcgcctatccgctcagccaacccgacgcggccctccagccctgtcacctcccacatctccaaggtgctttttggagaggatgacagcctgctgcgtgttgactgcatacgctacaaccgtgctgtccgtgatctgggtcctgtcatcagcacaggcctgctgcacctggctgaggatggggtgctgagtcccctggcgctgacagagggtgggaagggttcctcgccctccatcagaccaatccaaggcagccaggggtccagcagcccagtggagaaggaggtcgtggaagccacggacagcagagagaagacggggatggtgaggcctggcgagcccttgagtggggagaaatactcacccaaggagctgctggcactgctgaagtgtgtggaggctgagattgcaaactatgaggcgtgcctcaaggaggaggtagagaagaggaagaagttcaagattgatgaccagagaaggacccacaactacgatgagttcatctgcacctttatctccatgctggctcaggaaggcatgctggccaacctagtggagcagaacatctccgtgcggcggcgccaaggggtcagcatcggccggctccacaagcagcggaagcctgaccggcggaaacgctctcgcccctacaaggccaagcgccagtgaggactgctggccctgactctgcagcccactcttgccgtgtggccctcaccagggtccttccctgccccacttccccttttcccagtattactgaatagtcccagctggagagtccaggccctgggaatgggaggaaccaggccacattccttccatcgtgccctgaggcctgacacggcagatcagccccatagtgctcaggaggcagcatctggagttggggcacagcgaggtactgcagcttcctccacagccggctgtggagcagcaggacctggcccttctgcctgggcagcagaatatatattttacctatcagagacatctatttttctgggctccaacccaacatgccaccatgttgacataagttcctacctgactatgctttctctcctaggagctgtcctggtgggcccaggtccttgtatcatgccacggtcccaactacagggtcctagctgggggcctgggtgggccctgggctctgggccctgctgctctagccccagccaccagcctgtccctgttgtaaggaagccaggtcttctctcttcattcctcttaggagagtgccaaactcagggacccagcactgggctgggttgggagtagggtgtcccagtggggttggggtgagcaggctgctgggatcccatggcctgagcagagcatgtgggaactgttcagtggcctgtgaactgtcttccttgttctagccaggctgttcaagactgctctccatagcaaggttctagggctcttcgccttcagtgttgtggccctagctatgggcctaaattgggctctaggtctctgtccctggcgcttgaggctcagaagagcctctgtccagcccctcagtattaccatgtctccctctcaggggtagcagagacagggttgcttataggaagctggcaccactcagctcttcctgctactccagtttcctcagcctctgcaaggcactcagggtgggggacagcaggatcaagacaacccgttggagcccctgtgttccagaggacctgatgccaaggggtaatgggcccagcagtgcctctggagcccaggccccaacacagccccatggcctctgccagatggctttgaaaaaggtgatccaagcaggcccctttatctgtacatagtgactgagtggggggtgctggcaagtgtggcagctgcctctgggctgagcacagcttgacccctctagcccctgtaaatactggatcaatgaatgaataaaactctcctaagaatctcctgagaaaaaaaaaaaaaaaaa

SEQ ID NO: 20 - protein, 729 aa, Homo sapiens
  deubiquitinating agent BAP1
MNKGWLELESDPGLFTLLVEDFGVKGVQVEEIYDLQSKCQGPVYGFIFLFKWIEERRSRRKVSTLVDDTSVIDDDIVNNMFFAHQLIPNSCATHALLSVLLNCSSVDLGPTLSRMKDFTKGFSPESKGYAIGNAPELAKAHNSHARPEPRHLPEKQNGLSAVRTMEAFHFVSYVPITGRLFELDGLKVYPIDHGPWGEDEEWTDKARRVIMERIGLATAGEPYHDIRFNLMAVVPDRRIKYEARLHVLKVNRQTVLEALQQLIRVTQPELIQTHKSQESQLPEESKSASNKSPLVLEANRAPAASEGNHTDGAEEAAGSCAQAPSHSPPNKPKLVVKPPGSSLNGVHPNPTPIVQRLPAFLDNHNYAKSPMQEEEDLAAGVGRSRVPVRPPQQYSDDEDDYEDDEEDDVQNTNSALRYKGKGTGKPGALSGSADGQLSVLQPNTINVLAEKLKESQKDLSIPLSIKTSSGAGSPAVAVPTHSQPSPTPSNESTDTASEIGSAFNSPLRSPIRSANPTRPSSPVTSHISKVLFGEDDSLLRVDCIRYNRAVRDLGPVISTGLLHLAEDGVLSPLALTEGGKGSSPSIRPIQGSQGSSSPVEKEVVEATDSREKTGMVRPGEPLSGEKYSPKELLALLKCVEAEIANYEACLKEEVEKRKKFKIDDQRRTHNYDEFICTFISMLAQEGMLANLVEQNISVRRRQGVSIGRLHKQRKPDRRKRSRPYKAKRQ

SEQ ID NO: 21 - protein, 16 aa, Artificial Sequence
  Description of Artificial Sequencefirst branch of branched ubiquitin peptide
KSSTYKTVAKTGESVA

SEQ ID NO: 22 - protein, 19 aa, Artificial Sequence
  Description of Artificial Sequencedeubiquitinating agent HIS box
YXLXXXXXHXGXXXXXGHY

SEQ ID NO: 23 - protein, 17 aa, Artificial Sequence
  Description of Artificial Sequencedeubiquitinating agent CYS box
GXXXXXXXCXXXXXXQC

SEQ ID NO: 24 - protein, 37 aa, Artificial Sequence
  Description of Artificial Sequencedeubiquitinating agent HIS box
XXXXXXXHFXXXXXXXGXLXELDXXXXPXXXGXXXXX

SEQ ID NO: 25 - protein, 37 aa, Artificial Sequence
  Description of Artificial Sequencedeubiquitinating agent CYS box
XXXXXXXXXXXXXXXQXIXNXCXTXXXXXXXXXXXNX

(19) United States
(12) Patent Application Publication
(10) Pub. No.: US 2004/0053324 A1
(43) Pub. Date: Mar. 18, 2004

(54) Assays and compositions for identifying agents that modulate the activity of deubiquitinating agents

(76) Inventors: Brian Wong, San Mateo, CA (US); Todd R. Pray, San Francisco, CA (US); Jianing Huang, Foster City, CA (US); David Anderson, San Bruno, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/232,759

(22) Filed: Aug. 30, 2002

(51) Int. Cl. G01N 33/53; C12Q 1/37

(52) U.S. Cl. 435/7.1; 435/23

(57) ABSTRACT

Provided are methods and compositions for assaying for deubiquitinating agents that are enzymatic components of ubiquitin-mediated proteolysis and, their function, and agents that modulate the activity of such deubiquitinating agents.

### FIELD OF THE INVENTION

The invention relates to the field of ubiquitin-mediated proteolysis. In particular, the invention relates to methods and compositions for assaying for deubiquitinating agents that are enzymatic components of ubiquitin-mediated proteolysis and, more particularly, to methods and compositions for assaying for agents that modulate the activity of such deubiquitinating agents.

### BACKGROUND OF THE INVENTION

Ubiquitin is a highly conserved 76 amino acid protein expressed in all eukaryotic cells and is best known for its role in targeting proteins for degradation by the 26S proteasome. However, ubiquitin is involved in a variety of other cellular processes (Ciechanover and Schwartz (1994) FASEB J., 8:182-191; Wilkinson et al. (1995) Biochem., 34:14535-14546; Jentsch (1992) Trends Cell Biol. 2:98-103; Finley and Varshavsky (1985) Trends Biochem. Sci 10:343). The ubiquitination of target proteins involves the covalent ligation of the carboxyl terminus (C-terminus) of ubiquitin to the lysine side chains of target proteins, and is mediated by the enzymatic activity of at least three ubiquitin agents, including a ubiquitin activating agent, ubiquitin conjugating agent, and ubiquitin ligating agent. In this process, an isopeptide bond is formed between the carboxyl-terminal glycine of a ubiquitin and the ε-amino group of a lysine residue in a target protein and, thereby, monomers or oligomers of ubiquitin are attached to the target protein. Similarly, a lysine sidechain of one ubiquitin can be covalently ligated to another ubiquitin through the activity of a ubiquitin ligating agent. Thus, ubiquitin itself can serve as a ubiquitin substrate for ubiquitination. Further, a branched polyubiquitin chain can be formed by the sequential ligation of ubiquitin or polyubiquitin to another ubiquitin.

In addition, an unbranched polyubiquitin chain or linear fusion of two or more ubiquitin moieties, or ubiquitin fused to another polypeptide, can be formed via a peptide bond between the carboxyl-terminal glycine residue of a ubiquitin and the α-amino group at the terminus of another ubiquitin or another polypeptide. For example, ubiquitin is synthesized as a linear head-to-tail polyubiquitin precursor. Release of the monomeric ubiquitin by a deubiquitinating agent involves specific enzymatic cleavage between residues of the fused ubiquitin moieties of the polyubiquitin precursor. Each ubiquitin moiety is linked via an α-amino group, or a ubiquitin moiety is followed by a C-terminal peptide extension (Özkaynak et al. (1987) EMBO J. 6:1429-1439). The last ubiquitin moiety in many of these precursors is encoded with an extra C-terminal residue that can be removed to expose the active C-terminal Gly. Further, a chimeric ubiquitin can be constructed such that the ubiquitin moiety is fused to another protein and the ubiquitin moiety of the chimeric construct is specifically cleaved by a deubiquitinating agent at the precise junction where the ubiquitin moiety is fused to another protein (Bachmair et al. (1986) Science 234:179-186; Bachmair and Varshavsky (1989) Cell 56:1019-1032; Gonda et al., (1989) J. Biol. Chem. 26f4:16700-16712; and Varshavsky et al., U.S. Pat. Ser. No. 5,391,490).

Deubiquitination is catalyzed by deubiquitinating agents. For example, DeUbiquitinating (DUB) enzymes, are cysteine proteases that can hydrolyze either the ε-linked isopeptide bond or α-linked peptide bond at the C-terminus of a ubiquitin. In general, the deubiquitinating agents can specifically cleave ubiquitin complexes having the structure ubiquitin-N (Ub-N), where N is any number of leaving groups ranging in size, for example, from small amines and thiols to ubiquitin moieties and other proteins. These deubiquitinating agents can process polyubiquitin chains to generate free ubiquitin from precursor fusion polypeptides; affect pools of free ubiquitin by recycling branched chain ubiquitin and, also, remove ubiquitin from polyubiquitin or monomeric ubiquitin attached to a target protein (Johnston et al. (1999) EMBO 18:3877-3887; Johnston et al. (1997) EMBO 16:3787-3796), and specifically cleave the ubiquitin moiety of a chimeric ubiquitin fusion polypeptide (Dang et al. (1998) Biochemistry 37:1868-1879). In addition, ubiquitin-like proteases also process ubiquitin moieties in a similar manner to the DUB proteases (Olvera and Wool (1993) J. Biol. Chem. 268:17967-17974); Haas et al. (1996) Mol. Cell. Biol. 35:5385-5394; Matunis et al. (1996) J. Cell. Biol. 135:1457-1470; Narasimhan et al. (1996) J. Biol. Chem. 271:324-330; Mahajan et al. (1997) Cell 88:97-107).

In general, the deubiquitinating agents involved in the recycling of ubiquitin are thiol proteases that recognize the C-terminal domain or C-terminal residue of ubiquitin. The deubiquitinating agents may be divided into at least four classes: ubiquitin C-terminal hydrolases (UCH) (Pickart and Rose (1985) J. Biol. Chem. 261:10210-10217), ubiquitin-specific proteases (UBP; isopeptidases) (Tobias and Varshavsky (1991) 266:12021-12028), sentrin specific proteases (SENP) (Gong et al., J. Biol. Chem. (2000) 275:3355-3359 (2000)) and JAMM motif-containing proteases (JAMM-CP) (Deshuies, unpublished data).

UBPs contain six conserved domains, including a domain called the “CYS box” containing a conserved cysteine, a domain containing a conserved aspartic acid, and a domain called the “HIS box” containing a conserved histidine, which distinguish members of the UBP class. In particular, the domain containing the cysteine residue and domain containing the histidine residue have short sequences flanking these residues which are highly conserved in UBPs. Some members of the UBP class contain multiple ubiquitin binding sites, for example, DUB1, isoT, UBP3, Doa4, Tre2, and FAF. In addition, some members of the UBP class are transcriptionally induced in response to cytokines.

The members of the UCH class are also cysteine proteases. However, members of this class do not contain the six conserved domains characteristic of the UBP class. Members of the UCH class contain only one ubiquitin binding site and preferentially cleave ubiquitin from small molecules, for example, peptides and amino acids. In addition, these two classes of deubiquitinating agents share little sequence homology.

As with the UBP and UCH classes, members of the SENP class of deubiquitinating enzymes are cysteine proteases. However, the SENP proteases differ significantly from both the UBP and UCH proteases in structure and organization.

The JAMM-CP are the least well characterized class. They are believed to be metaloproteases and may have selectivity for NEDD8.

The function of the deubiquitinating enzymes include, for example, the disassembly of polyubiquitin to recycle ubiquitin, releasing ubiquitin from 26S proteasome substrates, releasing monomeric ubiquitin from ubiquitin fusion polypeptide precursors, the reversal of regulatory ubiquitination (e.g. the stabilization of protein substrates), the editing of ubiquitinated proteins that have been inappropriately ubiquitinated proteins, and regenerating active ubiquitin from adducts with small nucleophiles (e.g., glutathione) that may be generated by side reactions (Wilkinson and Hoschstrasser (1998) In Peters, J. M., Harris, J. R. and Finley, D. (Eds), Ubiquitin and the Biology of the Cell. Plenum Press, New York, N.Y., pp. 99-125). The end result of each of these activities can affect the level of free ubiquitin and other specific proteins in the cell (D'Andrea et al. (1998) Critical Reviews In Biochemistry and Molecular Biology 33:337-352).

For example, the yeast UBP14p deubiquitinating agent and its human homologue, isopeptidase-T, hydrolyze free polyubiquitin chains and promote the degradation of polyubiquitinated protein substrates by the 26S proteasome. One of the functions of isopeptidase-T in cells is thought to be the dissembly of unanchored polyubiquitin chains and sequential degradation of the polyubiquitin chains into ubiquitin monomers.

Further the yeast Doa4 deubiquitinating agent promotes ubiquitin-mediated proteolysis of cellular substrates. In particular, Doa4 appears to function in the hydrolysis of isopeptide-linked polyubiquitin chains from peptides that are the by-products of proteasome degradation. In addition, Doa4 appears to function in the cleavage of polyubiquitin from peptide degradation products. In general, the isopeptidases can produce free monomeric ubiquitin from branched or linear polyubiquitin chains, and from ubiquitin or polyubiquitin attached to target proteins or attached to degradation products or remnants of the ubiquitin substrate, for example, peptides or amino acids.

Deubiquitinating agents that promote stabilization of substrates include the FAF protein, which deubiquitinates and rescues a ubiquitin-conjugated target protein from degradation by the proteasome. The PA700 isospeptidase, another deubiquitinating agent, also prevents proteasome degradation apparently by removing ubiquitin moieties, one at a time, beginning from the distal end of a polyubiquitin chain.

Deubiquitinating agents are also associated with growth control. For example, the mammalian oncoprotein Tre-2 is a member of the UBP class of deubiquitinating agents. The truncated UPB lacking the histidine domain and lacking deubiquitinating activity is the transforming isoform of the Tre-2 oncoprotein, while, the full-length Tre-2 protein has deubiquitinating activity but does not have transforming activity. The full-length Tre-2 protein is thought to act as an intracellular growth suppressor. DUB-1 is another UBP that is thought to regulate cellular cellular processes. DUB-1 is induced by interleukin-3 stimulation. In general, members of this class of deubiquitinating agents are thought to be responsive to cytokines. Further, DUB-2, another member of this class, is induced by interleukin-2. (Zhu et al. (1997) Journal of Biological Chemistry 272:51-57). This class of deubiquitinating agents may deubiquitinate cell surface growth factor receptors, thereby, prolonging receptor half life and amplifying growth signals; and may also deubiquitinate proteins involved in signal transduction and proteins that are cell cycle regulators, for example, cyclins and cyclin-CDK inhibitors.

UBPs are known to be involved in the chromatin regulatory process and transcriptional silencing. For example, UBP-3 forms a complex with SIR-4, a trans-acting factor that is required for activating and maintaining transcriptional silencing. Consequently, UBP-3 is thought to act as an inhibitor of transcriptional silencing by stabilizing an inhibitor or by removing a positive regulator. As a further example, the murine UNP protooncogene encodes a nuclear ubiquitin protease that when overexpressed results in oncogenic transformation in NIH3T3 cells. The cDNA corresponding to the human homologue of the murine UNP protooncogene was cloned and mapped to a chromosomal region frequently rearranged in human tumor cells. Moreover, the levels of the protooncogene were elevated in small cell tumors and adenocarcinomas of the lung. Thus, the this protooncogene may have a causitive role in the neoplastic process (Gray et al. (1995) Oncogene 10:2179-2183).

Another UBP designated UBP-43, was cloned from a leukemia fusion protein in AML1-ETO Knockout mice, and has been shown to function in hematopoitic cell differentiation. The overexpression of this gene blocks cytokine-induced terminal differentiation of monocytic cells (Liu et al. (1999) Molecular and Cellular Biology 19:3029-3038).

Thus, as described above, deubiquitinating agents are key determinants of the ubiquitin-mediated proteolytic pathway that results in the degradation of targeted proteins and regulation of a variety of cellular processes. Consequently, agents that modulate the activity of such deubiquitinating agents may be used to upregulate or downregulate specific molecules in the cell involved in signal transduction. Thus, diseases can be treated by the upregulation or downregulation of such molecules to modulate (e.g., stimulate or inhibit) specific cellular responses or processes, and drugs for treatment of diseases can be designed based such modulation.

Due to the importance of ubiquitin-mediated proteolysis in cellular processes there is a need for a rapid and simple means for identifying deubiquitinating agents that are catalytic components of ubiquitin-mediated proteolysis, and for identifying modulating agents that modulate the activity of such deubiquitinating agents. Thus, an object of the present invention is to provide methods of assaying for deubiquitinating agents that are catalytic components of ubiquitin-mediated proteolysis and, more particularly, methods of assaying for agents that modulate the activity of such deubiquitinating agents.

### SUMMARY OF THE INVENTION

In accordance with the above objects, the present invention provides cell-free and cell-based methods and compositions for assaying for deubiquitinating agents that are enzymatic components of ubiquitin-mediated protein regulation. More particularly, the present invention provides cell-free and cell-based methods and compositions for assaying for an agent that modulates the activity of a deubiquitinating agent. Specifically, the methods of the present invention are directed to identifying deubiquitinating agents such as ubiquitin-specific proteases(UBPs) and ubiquitin C-terminal hydrolases (UCHs); and to identifying agents that modulate the activity of these deubiquitinating agents, for example, the binding, cleavage, or release of a ubiquitin moiety from a ubiquitin complex. In one aspect, the invention provides assaying methods that do not require a ubiquitin target protein. In the methods of the present invention, to assay for deubiquitinating activity, a candidate deubiquitinating agent and ubiquitin complex is combined in a reaction mixture in vitro or in a cell in vivo and assayed for deubiquitinating activity, for example, the specific cleavage or release of the ubiquitin moiety from the ubiquitin complex by the candidate deubiquitinating agent. Further, in the methods of the present invention, to assay for the modulation of deubiquitinating activity, a candidate modulating agent, deubiquitinating agent, and ubiquitin complex can be combined in a reaction mixture in vitro or in a cell in vivo and assayed for the modulation of deubiquitinating activity.

In one aspect, the invention provides methods of assaying for a candidate modulating agent that modulates the cleavage of a ubiquitin complex by a deubiquitinating agent, the method comprising the steps of: a) combining a candidate modulating agent, a ubiquitin complex, and a deubiquitinating agent; and b) assaying for the modulation of the cleavage by the candidate modulating agent.

In another aspect, the invention provides methods of assaying for a candidate modulating agent that modulates the cleavage of a ubiquitin complex in a cell by a deubiquitinating agent, the method comprising the steps of: a) providing a cell comprising a deubiquitinating agent and a ubiquitin complex; b) introducing into the cell a candidate modulating agent; and c) assaying for the modulation of the cleavage by the candidate modulating agent. In a further aspect, the cell is a mammalian cell.

The following are further aspects of the methods and compositions of the present invention for assaying, in vitro or in vivo, for a candidate modulating agent that modulates the cleavage of a ubiquitin complex by a deubiquitinating agent.

In an aspect of the methods and compositions of the present invention, the deubiquitinating agent is a mammalian UBP, UCH, SENP or JAMM-CP and in a further aspect, the deubiquitinating agent is a derivative of a UBP, UCH, SENP or JAMM-CP. In another aspect, the deubiquitinating agent comprises a subsequence of the full length amino acid sequence of a UBP, UCH, SENP or JAMMCP. In a further aspect, the subsequence of the full length amino acid sequence has deubiquitinating activity, for example, specific binding, cleavage or release of a ubiquitin moiety in a ubiquitin complex.

In an aspect of the methods and compositions of the present invention, the ubiquitin complex comprises the general structure Ub-N, where Ub is a ubiquitin moiety and attached to N via an isopeptide or peptide bond, and N can be any number of leaving groups ranging from a small amine or thiol to another ubiquitin moiety or another protein. For example, N can be a ubiquitin substrate; and Ub-N can be a cleavable ubiquitin fusion polypeptide. Examples of cleavable ubiquitin fusion polypeptides include, but are not limited to, a ubiquitin moiety fused to another ubiquitin moiety or another polypeptide; or a branched ubiquitin peptide. The ubiquitin moiety can comprise a full-length ubiquitin or ubiquitin-like polypeptide or a peptide having a subsequence of the full-length amino acid sequence of a ubiquitin or ubiquitin-like polypeptide that can be specifically cleaved by a deubiquitinating agent. In one aspect, the ubiquitin moiety comprises the C-terminus of a ubiquitin moiety. Examples of ubiquitin substrates include, but are not limited to, a ubiquitin agent, a target protein, or a mono- or poly-ubiquitin moiety which is may be attached to a ubiquitin agent or target protein.

In some aspects of the methods and compositions of the present invention, the ubiquitin agent is a ubiquitin conjugating agent (E2) or a ubiquitin ligating agent (E3). In other aspects, the target protein is a mammalian target protein, and in further aspects, the target protein is a human target protein. In other aspects, the ubiquitin moiety is mammalian, preferably human. In another aspect, the ubiquitin moiety is a ubiquitin derivative or comprises a subsequence of the full length amino acid sequence of a ubiquitin polypeptide. In some aspects, the ubiquitin moiety comprises a label, and in further aspects, the label comprises an epitope tag. In other aspects, at least a first and a second ubiquitin moiety is used, wherein the first and second ubiquitin moieties comprise different fluorescent labels, and wherein the labels form a fluorescence resonance energy transfer (FRET) pair.

In another aspect, the ubiquitin complex comprises a poly-ubiquitin chain, and the poly-ubiquitin chain comprises at least two ubiquitin moieties. Also in another aspect, the ubiquitin complex comprises a poly-ubiquitin chain, and the poly-ubiquitin chain comprises a first ubiquitin moiety and a second ubiquitin moiety. In a further aspect, the first ubiquitin moiety comprises a first label and the second ubiquitin moiety comprises a second label. In a further aspect, the first ubiquitin moiety comprises a first FRET label and the second ubiquitin moiety comprises a second FRET label. In a further aspect, the first ubiquitin moiety comprises a FRET label and the second ubiquitin moiety comprises a Quencher.

In another aspect, the ubiquitin complex in the methods of the present invention is formed by combining a ubiquitin moiety and ubiquitin substrate in a reaction mixture in vitro or in a cell in vivo. In another aspect, the ubiquitin complex is purified and the purified ubiquitin complex is combined in a reaction mixture in vitro with a deubiquitinating agent and assayed for deubiquitinating activity and, in a further aspect, the reaction mixture further comprises a candidate modulating agent and then assayed for the ability to modulate deubiquitinating activity.

In another aspect, the ubiquitin complex comprises a target protein comprising at least one ubiquitin moiety. Also in another aspect, the ubiquitin complex comprises a ubiquitin agent comprising at least one ubiquitin moiety. In a further aspect, the ubiquitin agent is an E2 or an E3.

In a further aspect, the target protein comprises a first FRET label and the ubiquitin moiety comprises a second FRET label. In a further aspect, one of the target protein and the ubiquitin moiety comprises a FRET label and the other comprises a Quencher. Also in a further aspect, the target protein is linked to a reporter protein. In another aspect, the target protein comprises an attachment moiety, and in a further aspect, the target protein is provided on a solid support, for example a microtiter plate or a bead.

In another aspect of the invention, the ubiquitin complex comprises a ubiquitin agent bound to a ubiquitin moiety via an isopeptide bond, wherein the ubiquitin agent comprises a first FRET label and the ubiquitin moiety comprises a second FRET label. Alternatively, one of the ubiquitin moiety and the ubiquitin agent comprises a FRET label and the other comprises a Quencher.

In another aspect, one member of the ubiquitin complex comprises an attachment moiety. In another aspect of the invention, the ubiquitin complex is provided on a solid support, for example, a microtiter plate or a bead, preferably via attachment of at least one less than the total number of members of the ubiquitin complex.

In another aspect, the ubiquitin complex is a cleavable ubiquitin fusion polypeptide. In a further aspect, the cleavable ubiquitin fusion polypeptide is a branched ubiquitin peptide comprising a first branch and a second branch wherein: a) the first branch comprises, from amino to carboxyl terminus: i) flanking amino acids 1, 2, and 3; ii) a branched lysine, K; and iii) flanking amino acids 4, 5, and 6, wherein flanking amino acids 1, 2, 3, 4, 5, and 6 are selected from amino acids flanking the lysine in a ubiquitin substrate and located within about 10 amino acids from the lysine in the ubiquitin substrate; and b) the second branch comprises an amino acid sequence encoded by the C-terminus of a ubiquitin moiety, wherein the amino acid sequence is at least about 4-6 amino acids in length, and wherein the second branch is joined to the branched lysine of the first branch. In another aspect, the amino acid sequence of the second branch is, from amino to carboxyl terminus, LRLRGG. Also, in another aspect, the first branch comprises the amino acid sequence, from amino to carboxyl terminus, KSSTYKTVA, wherein K is the branch point.

In further aspect, the cleavable ubiquitin fusion polypeptide comprises at least one tag. In another aspect, the cleavable ubiquitin fusion polypeptide comprises a first tag and a second tag. Also in a further aspect, the first tag is at the amino terminus of the cleavable ubiquitin fusion polypeptide and the second tag is at the carboxyl terminus of the ubiquitin moiety. In another aspect, the first tag is a first label and the second tag is a second label. Also, in another aspect, first label is a first FRET label and the second label is a second FRET label; or the label at one terminus is a FRET label and the label at the other terminus is a Quencher of the FRET label. In another aspect, the tag at one terminus comprises a Flag tag and the tag at the other terminus comprises a His tag.

In another aspect, the cleavable ubiquitin fusion polypeptide comprises a first ubiquitin moiety comprising a first tag bound, via a peptide bond or an isopeptide bond, to a second ubiquitin moiety comprising a second tag. In another aspect, the first tag is at the amino terminus of the first ubiquitin moiety and the second tag is at the carboxyl terminus of the second ubiquitin moiety and in a further aspect, either the first tag or the second tag is a His tag; or the first tag or the second tag is a GST tag. In another aspect, the cleavable ubiquitin fusion polypeptide comprises a ubiquitin moiety operably linked to a reporter protein.

In a further aspect, the cleavable ubiquitin fusion polypeptide comprises a ubiquitin moiety bound, via a peptide bond or an isopeptide bond, to a reporter protein. In another aspect, the reporter protein is beta-galactosidase or a fluorescent reporter protein, for example, Green Fluorescent Protein (GFP) and more specifically, Green Fluorescent Protein (GFP) of a renilla species.

In another aspect, assaying is by FACS. In another aspect, assaying is by high pressure liquid chromatography (HPLC), for example, reverse phase HPLC. Assaying can also be done by capillary electrophoresis, fluorescence analysis (e.g. FRET, as described below) and by mass spectrometry.

In one aspect, the candidate modulating agent is a mutant cDNA encoding a catalytically inactive polypeptide. Examples of such catalytically inactive polypeptides include, but are not limited to, catalytically inactive deubiquitinating agents and, more specifically, catalytically inactive UBP, UCH, SENP or JAMM-CP.

In another aspect, the candidate modulating agent is an RNA, for example an antisense RNA or siRNA. In a further aspect, the siRNA cleaves RNA encoding a deubiquitinating agent, for example a UBP, UCH, SENP or JAMM-CP.

In another aspect, the candidate modulating agent is a polypeptide. In a further aspect, the polypeptide is a peptide having at least 4 and up to 20 or more amino acids and may be a cyclic peptide. Also in a further aspect, the polypeptide is a catalytically inactive polypeptide. Examples of catalytically inactive polypeptides include, but are not limited to, catalytically inactive deubiquitinating agent and, more specifically a catalytically inactive UBP, UCH, SENP or JAMM-CP

In a most preferred embodiment, the candidate agent is an organic molecule.

In addition, the invention provides a method comprising providing a library of cells comprising a library of nucleic acids comprising nucleic acid encoding at least one negative effector of a deubiquitinating agent, screening the library of cells for an altered phenotype as compared to control cells, isolating at least one altered cell with the altered phenotype and identifying the negative effector in the altered cell. The negative effector may be a siRNA, an antisense nucleic acid, a peptide, including a cyclic peptide, or a variant of a deubiquitinating agent, including a truncated variant or fragment of the deubiquitinating agent.

In a still further aspect, the invention provides a method of identifying and a phenotype modulated by a deubiquitinating agent. The method comprises contacting a cell with a negative effector of a deubiquitinating agent and screening the cell for an altered phenotype. The negative effector may be an siRNA or antisense nucleic acid directed against a nucleic acid encoding the deubiquitinating agent. The negative effector may be a dominant negative variant of a deubiquitinating agent.

### BRIEF DESCRIPTION OF THE DRAWINGS

FIG. 1 depicts the results of an assay using Ubiquitin-AMC cleavable ubiquitin fusion polypeptide as a ubiquitin complex, as described in Example 1. The results indicate that Ub-AMC was hydrolyzed with release of highly fluorescent AMC, which was detected by a fluoro-scanner.

FIG. 2 depicts the results of an assay using purified poly-Ubiquitin2-7 cleavable ubiquitin fusion polypeptide as a ubiquitin complex, as described in Example 2. The results indicate the polyubiquitin ladders of the ubiquitin complex were reduced, and ubiquitin moiety was cleaved and released from the ubiquitin complex by Gst-UbpM or His-UbpM, thereby, forming free ubiquitin moiety.

FIG. 3 depicts the results of an assay using linear Flag-ubiquitin-His(6) cleavable ubiquitin fusion polypeptide as a ubiquitin complex and UbpM as the deubiquitinating agent, as described in Example 3. The results indicate that the Flag-ubiquitin moiety was cleaved and released from the ubiquitin complex and thus the ni-plate by Gst-UbpM or His-UbpM leaving only His(6) attached to the plate, and that any uncleaved Flag-ubiquitin-His attached to the Ni-plate was detected by anti-Flag.

FIG. 4 depicts the results of an assay using auto-ubiquitinated ligase containing poly-ubiquitin chain as a ubiquitin substrate and UbpM as the deubiquitinating agent, as described in Example 4. The results indicate that Flag-ubiquitin of the polyubiquitin chain was cleaved and released from the ubiquitin complex by the deubiquitinating agent, thereby forming free Flag-ubiquitin moiety, and the remaining poly-Flag-ubiquitin attached to the ni-plate was detected by an anti-Flag immunoassay.

FIG. 5 depicts an example of the structure and sequence of a branched ubiquitin peptide. The branched lysine is depicted as “K” (in bold).

FIG. 6 depicts an example of the structure and sequence of a branched ubiquitin peptide SAR useful as a modulator, for example an inhibitor, of deubiquitinating activity; and also depicts how to screen for and make a mutated branched ubiquitin peptide for inhibitor development.

FIG. 7 depicts fluorescein dequenching upon cleavage of poly-Ub by UbpM.

FIG. 8 depicts dose-dependence of UbpM cleavage of FRET-quenched APC2/APC11-(poly-Ub)

FIGS. 9A and 9B show the nucleic acid sequence and amino acid sequence, respectively, for deubiquitinating agent UbpM (USP16).

FIGS. 10A-10C show the nucleic acid sequence (10A-10B) and amino acid sequence (10C), for deubiquitinating agent USP-25.

FIGS. 11A and 11B show the nucleic acid sequence and amino acid sequence, respectively, for deubiquitinating agent Yuh1 homolog (ub c-terminal esterase L1).

FIGS. 12A and 2B show the nucleic acid sequence and amino acid sequence, respectively, for deubiquitinating agent Unph Protooncogene (USP4).

FIG. 13A and 13B show the nucleic acid sequence and amino acid sequence, respectively, for deubiquitinating agent BRAP.

FIGS. 14A-14C show the nucleic acid sequence (14A-14B) and amino acid sequence (14C) dor deubiquitiniating agent BAP1.

### DETAILED DESCRIPTION OF THE INVENTION

The present invention provides cell based and cell free methods and compositions for assaying for deubiquitinating agents and their function. More particularly, the present invention provides methods and compositions for assaying for agents that modulate the activity of a deubiquitinating agent and, thereby, its function. Specifically, the methods of the present invention are directed to identifying deubiquitinating agents such as ubiquitin-specific proteases (UBPs), ubiquitin C-terminal hydrolases (UCHs) , sentrin specific proteases (SENP) and JAMM motif-containing proteases (JAMM-CP); and to identifying agents that modulate the activity of these deubiquitinating agents.

The advantages of the present invention include providing methods and compositions for assaying for the activity of deubiquitinating agents in one reaction vessel thus obviating the need for subsequent steps, for example, for separating and purifying the products of the reaction. Consequently, this approach allows multi-well array analysis and high throughput screening techniques for agents that modulate the activity of deubiquitinating agents. In addition, the present invention provides methods and compositions that allow the analysis of many different deubiquitinating agents and modulators of deubiquitinating agents, without requiring prior identification of specific target proteins. In particular, the present invention provides methods that allow the analysis of different deubiquitinating agents and modulators of deubiquitinating agents in the absence of a target protein. Alternatively, the present invention provides methods that allow the analysis of deubiquitinating agents and modulators of deubiquitinating agents in the presence of a target protein.

Other advantages of the methods and compositions of the present invention include the use of ubiquitin fusion polypeptides and branched ubiquitin peptides that are specifically cleaved by deubiquitinating agents. The assays can be multiplexed using reverse phase high pressure liquid chromatography and mass spectrometry for detection of deubiquitinating activity and the modulation of such activity in the presence of a candidate modulating agent. More particularly, the assays can be used for detecting the specific products of deubiquitination. Further, the use of these ubiquitin complexes in the methods of the present invention provides assays for the facile identification of structure-activity relationships by varying individual amino acids in the branched ubiquitin peptide. Thus, the methods and compositions of the present invention are particularly useful for high throughput screening because many different reactions can be performed concurrently and, further, for the precise site of cleavage by the deubiquitinating agent.

In preferred embodiments, the invention provides methods of assaying for the deubiquitinating activity of a deubiquitinating agent, or candidate deubiquitinating agent, by combining the agent with a ubiquitin complex in a reaction mixture in vitro or in a cell in vivo and assaying for deubiquitinating activity (e.g., for identifying and/or characterizing deubiquitinating agents). In other preferred embodiments, the invention provides methods of assaying for the modulation of deubiquitinating activity by a candidate modulating agent by combining a deubiquitinating agent, ubiquitin complex, and candidate modulating agent in a reaction mixture in vitro or in a cell in vivo and assaying for the modulation of deubiquitinating activity.

Accordingly, the invention provides methods of assaying for a candidate modulating agent that modulates the cleavage of a ubiquitin complex by a deubiquitinating agent, the method comprising the steps of: a) combining a candidate modulating agent, a ubiquitin complex, and a deubiquitinating agent; and b) assaying for modulation of the cleavage by the candidate modulating agent. The order of combining the deubiquitinating agent, ubiquitin complex, and candidate modulating agent can be varied. For example, the order of combining can be varied so that the reactants of the deubiquitinating reaction are sequentially or concurrently combined in the reaction mixture, in vitro. In a preferred embodiment, a ubiquitin complex is combined with a deubiquitining agent, or both a deubiquitinating agent and a candidate agent, in a reaction mixture and then assayed for deubiquitinating activity or modulation of this activity, respectively. In a preferred embodiment, the ubiquitin complex is purified prior to use in the assays of the present invention. In another preferred embodiment, the candidate modulating agent is first combined with the ubiquitin complex, followed by combining of the deubiquitinating agent in the reaction mixture, in vitro, and assayed for modulation of deubiquitinating activity. For example, the ubiquitin complex can be preincubated with the candidate modulating agent in a reaction mixture prior to addition of the deubiquitinating agent; or the candidate modulating agent can be preincubated with the deubiquitinating agent prior to addition of the ubiquitin complex.

In another preferred embodiment, the invention provides methods of assaying for a candidate modulating agent that modulates the cleavage of a ubiquitin complex in a cell by a deubiquitinating agent, the method comprising the steps of: a) providing a cell comprising a deubiquitinating agent and a ubiquitin complex; b) introducing into the cell a candidate modulating agent; and c) assaying for modulation of the cleavage by the candidate modulating agent. In a preferred embodiment, the cell is a mammalian cell. The cell can be a native cell expressing a deubiquitinating agent. In a preferred embodiment, the cell is a recombinant cell where nucleic acid encoding a deubiquitinating agent, a candidate modulating agent, a ubiquitin agent, and/or a ubiquitin substrate can be introduced into the cell and expressed. The deubiquitinating agent, ubiquitin complex, and a candidate modulating agent can be introduced into a host cell and expressed inducibly or consitutively, or transiently or stably using the recombinant methods described herein. Nucleic acids encoding the deubiquitinating agent, ubiquitin complex, and candidate modulating agents can be introduced into the cell sequentially or concurrently, in trans or in cis. The order of combining can be varied so that the reactants of the deubiquitinating reaction are introduced into the cell and/or expressed in the cell sequentially or concurrently. In one embodiment, the constituents for forming the ubiquitin complex, a ubiquitin moiety and, optionally, a ubiquitin substrate and one or more including ubiquitin agents, are first introduced to form the ubiquitin complex, followed by introduction of a deubiquitinating agent. In a preferred embodiment, the reactants for forming the ubiquitin complex (e.g., ubiquitin moiety and ubiquitin substrate) are first introduced to form the ubiquitin complex, followed by introduction of a deubiquitinating agent and a candidate modulating agent. In another preferred embodiment, the candidate modulating agent is first combined with a ubiquitin complex, followed by combining of the deubiquitinating agent.

As used herein, “deubiquitinating activity” refers to any biological activity associated with a deubiquitinating agent and described herein or known in the art, for example, a cellular process, catalytic property, and more specifically, the binding, release, or cleavage of a ubiquitin moiety from a ubiquitin complex. Examples of cellular processes involving deubiquitinating agents include, but are not limited to, the disassembly of polyubiquitin to recycle ubiquitin; releasing of ubiquitin from 26S proteasome substrates, releasing of monomeric ubiquitin from ubiquitin fusion polypeptide precursors, reversal of regulatory ubiquitination, editing of ubiquitinated proteins that have been inappropriately ubiquitinated proteins, and regeneration of active ubiquitin from adducts with small nucleophiles (e.g., glutathione) that may be generated by side reactions (Wilkinson and Hoschstrasser, 1998, incorporated herein by reference). Generally, such deubiquitinating activity affects the level of free ubiquitin and other specific proteins in the cell (D'Andrea et al. (1998) Critical Reviews In Biochemistry and Molecular Biology 33:337-352, incorporated herein by reference).

A preferred deubiquitinating activity is the release of a ubiquitin moiety from either a polyubiquitin chain or protein substrate.

Deubiquitinating activity, or the modulation of deubiquitinating activity, can be detected and measured using the methods described herein or known in the art (e.g., see Sjolander et al. (1991) Anal. chem. 63:2338-2345; Szabo et al. (1995) Curr. Opin. Struct. Biol. 5:699-705; and U.S. Pat. Ser. No. 6,329,171 to Kapeller-Libermann et al.; Zhu et al. (1997) Journal of Biological Chemistry 272:51-57, Mitch et al. (1999) American Journal of Physiology 276: C 1132-C1138; Liu et al. (1999) Molecular and Cell Biology 19:3029-3038; Ciechanover et al. (1994) The FASEB Journal 8:182-192; Chiechanover (1994) Biol. Chem. Hoppe-Seyler 375:565-581; Hershko et al. (1998) Annual Review of Biochemistry 67:425-479; Swartz (1999) Annual Review of Medicine 50:57-74, Ciechanover (1998) EMBO Journal 17:7151-7160; and D'Andrea et al. (1998) Critical Reviews in Biochemistry; and Molecular Biology 33:337-352), all of which are expressly incorporated herein by reference. Examples of assays for the detection and measurement of deubiquitinating activity include, but are not limited to, the disappearance of ubiquitinated polypeptides (i.e., ubiquitin complexes), including decrease in the amount of polyubiquitin or ubiquitinated protein or protein remnant or fragment; appearance of intermediate and end products of deubiquitining activity, e.g., the appearance of free ubiquitin monomers or released or cleaved ubiquitin moiety; general or specific protein turnover; binding to ubiquitin moiety; binding to ubiquitinated polypeptides (i.e., ubiquitin complexes); and stabilization of specific proteins.

As used herein, “specific” when used with reference to the deubiquitinating activity of a deubiquitinating agent, for example, “specific binding”, “specific release”, “specific cleavage” of a ubiquitin moiety of ubiquitin complex or “specific deubiquitination” of a ubiquitin complex, refers to an activity dependent on the presence of the deubiquitinating agent; or dependent on a biological property that is characteristic of, or associated with, the deubiquitinating agent; or dependent on a biologically active domain of the deubiquitinating agent; and more preferably dependent on a catalytic property of the deubiquitinating agent.

As used herein, “deubiquitinating agent” refers to an agent that has deubiquitinating activity, generally, a deubiquitinating enzyme. In general, the deubiquitinating agents of the present invention can be prepared using synthetic or recombinant methods described herein or known in the art. Preferably, the deubiquitinating agent is a polypeptide of a full-length protein or peptide encoding a subsequence of the full-length amino acid sequence of a full-length protein having deubiquitinating activity. Preferably, the peptide is a biologically active peptide, and more preferably, the biologically active peptide has a deubiquitinating activity. In a preferred embodiment, the deubiquitinating agent is a mammalian UBP, UCH, SENP or JAMM-CP. In another preferred embodiment, the deubiquitinating agent is a derivative of a UBP, UCH, SENP or JAMM-CP (e.g., variants of deubiquitinating agents having amino acid substitutions, insertions, and deletions). In another preferred embodiment, the deubiquitinating agent comprises a subsequence of the full length amino acid sequence of a UBP, UCH, SENP or JAMM-CP. In a further aspect, the subsequence of the full length amino acid sequence has deubiquitinating activity. Variants and derivatives of deubiquitinating agents are contemplated for use in the methods of the present invention and are included in the definition of deubiquitinating agent. Also, fragments or subsequences of a deubiquitinating agent, for example a full-length deubiquitinating agent, are also contemplated for used in the methods of the present invention and are included in the definition of a deubiquitinating agent.

As used herein, “ubiquitin complex” refers to a polypeptide comprising a ubiquitin moiety and a ubiquitin substrate or another polypeptide. In a preferred embodiment, the ubiquitin moiety of the ubiquitin complex is specifically recognized, bound, or cleaved by a deubiquitinating agent. In a preferred embodiment, the ubiquitin complex comprises an amino acid sequence from the C-terminus of a ubiquitin moiety or biologically active domain of a ubiquitin moiety. In a another preferred embodiment, the ubiquitin complex comprises the general structure Ub-N, where Ub is a ubiquitin moiety and attached to N via an isopeptide or peptide bond, and N can be any number of leaving groups ranging from a small amine or thiol to another ubiquitin moiety or another protein. Further examples of N include, but are not limited to, a target protein, ubiquitin agent, reporter protein, or other protein (e.g., a drug or polypeptide attached to a drug). In a preferred embodiment, Ub-N is a cleavable ubiquitin fusion polypeptide. Examples of cleavable ubiquitin fusion polypeptides include, but are not limited to, a ubiquitin moiety fused to another ubiquitin moiety or another polypeptide; or a branched ubiquitin peptide. In another embodiment, Ub-N is specifically cleaved by a deubiquitinating agent at the C-terminus of Ub and, preferably, the cleavage site is at the junction between Ub and N.

In a preferred embodiment, N is a ubiquitin substrate. Examples of ubiquitin substrates include, but are not limited to, a ubiquitin agent, a target protein, or a mono- or poly-ubiquitin moiety which is preferably attached to a ubiquitin agent or target protein. The ubiquitin moiety of Ub-N can comprise a full-length ubiquitin polypeptide or a peptide encoding a subsequence of the full-length amino acid sequence of a ubiquitin polypeptide that can be specifically cleaved by a deubiquitinating agent. In one aspect, the ubiquitin moiety comprises the amino acid sequence or peptide domain of the C-terminus of a ubiquitin moiety.

As used herein with reference to a ubiquitin moiety, “C-terminus” refers to the peptide domain or amino acid sequence of a ubiquitin moiety that has deubiquitinating activity and more particularly, is specifically recognized, bound, or cleaved by a deubiquitinating agent.

As used herein, “ubiquitin fusion polypeptide” refers to a ubiquitin moiety fused to another polypeptide or peptide. Further, as used herein, “cleavable ubiquitin fusion polypeptide” refers to a ubiquitin fusion polypeptide that can be specifically bound, released, or cleaved by a deubiquitinating agent. The fusion may be direct or indirect (e.g., a linker may be used to fuse a ubiquitin moiety to another polypeptide or peptide).

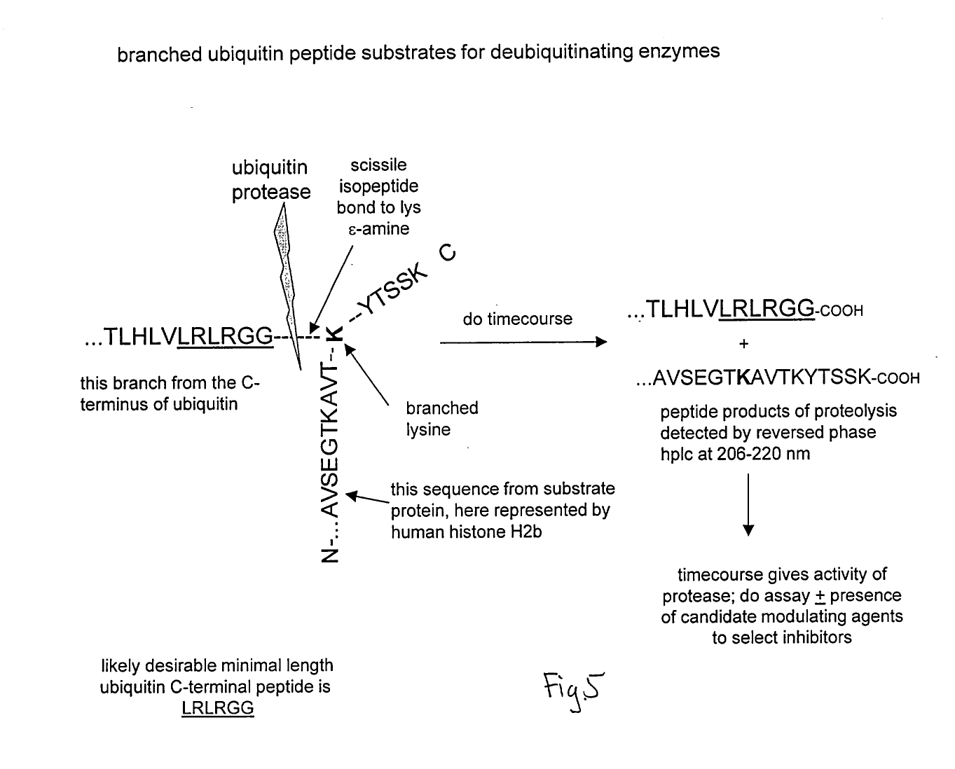

In another preferred embodiment, the cleavable ubiquitin fusion polypeptide is a branched ubiquitin peptide. As used herein, “branched ubiquitin peptide” refers to a peptide comprising at least a first branch and a second branch: where the first branch comprises from amino to carboxyl terminus, a) preferably up to 20 flanking amino acids, and more preferably 3-6 flanking amino acids; b) a branched lysine designated “K” (in bold); and c) preferably up to 20 flanking amino acids, and more preferably 3-6 flanking amino acids; and where the second branch comprises an amino acid sequence encoded by the C-terminus of a ubiquitin moiety, and the length of the amino acid sequence extending from at least 3-20 amino acids up to the length of a full-length ubiquitin protein; and where the second branch is joined to the branched lysine “K” via a scissile isopeptide bond to the ε-amine of the branched lysine “K”, and the scissile isopeptide bond is cleavable by a deubiquitinating agent. See, for example, the preferred embodiment depicted in FIGS. 5 and 6.

As used herein with reference to a branched ubiquitin peptide, “flanking amino acids” refer to amino acid residues in the first branch of the branched ubiquitin peptide which are located immediately adjacent to the branched lysine “K” and either preceding or following “K”. Preferably the flanking amino acids are selected from amino acid residues that: 1) precede or follow the lysine of a ubiquitin substrate; and 2) preferably, are located within 20-50 amino acids of the lysine of a ubiquitin substrate. In a preferred embodiment, the branched ubiquitin peptide has up to 20 flanking amino acids; and more preferably, the branched ubiquitin peptide had 1-6 flanking amino acids.

In a preferred embodiment, the second branch of the branched ubiquitin peptide comprises the amino acid sequence, from amino to carboxyl terminus, LRLRGG as depicted in FIGS. 5 and 6.

In another preferred embodiment, the first branch of the branched ubiquitin peptide comprises the amino acid sequence, from amino to carboxyl terminus, KSSTYKTVAKTGESVA as depicted in FIGS. 5 and 6.

In some preferred embodiments, the branched ubiquitin peptide is a minimal peptide sufficient for proteolytic activity by a deubiquitinating agent, e.g., sufficient for cleavage by a deubiquitinating agent. An example of such a minimal peptide is a branched ubiquitin peptide containing a scissile bond located at the isopeptide ubiquitin-lysine bond.

As will be appreciated by those in the art, there are a variety of methods to detect cleavage of the ubiquitin peptide. For example, in a preferred embodiment, cleavage of the branched ubiquitin peptide is detected by HPLC, including reverse phase HPLC, electrophoresis (including capillary electrophoresis), and fluorescence analysis (e.g. FRET analysis, as outlined below) etc. The branched ubiquitin peptides of the present invention can be prepared by synthesis or by the recombinant methods described herein, and can be used and detected without additional chromophores or fluorophores since the ubiquitin branched peptide and products can be detected by peptide bond absorbance. In a preferred embodiment, to assay for the modulation of deubiquitinating activity by a candidate modulating agent, the candidate modulating agent is combined and optionally preincubated with the branched ubiquitin peptide in the reaction mixture, thereafter, the deubiquitinating agent is combined in the reaction mixture and the modulation of deubiquitinating activity, over a time course is directly measured (see e.g., FIG. 5). Such methods of the present invention can be easily automated, and permit facile development of structure-activity relationships by varying individual amino acids (“X”) at any position in the two branches of the substrate, or varying the branched lysine (to another amino-containing amino acid, e.g., ornithine).

Thus, the assays of the present invention can be used to rapidly and easily perform high throughput searches for deubiquitinating activity or modulators of deubiquitinating activity. More particularly, the methods of the present invention can be used to assay for inhibitors of deubiquitinating activity. For example, the assays can be multiplexed using reverse phase HPLC-mass spectrometry or capillary electrophoresis for detection, thereby, allowing examination of the cleavage of equimolar mixtures of multiple ubiquitin branched peptides, with many different natural X amino acids in a single X position, e.g., 19 different natural X amino acids. Such amino acid variation can be easily prepared and analyzed because: 1) each ubiquitin branched peptide and any one of the peptide products of the deubiquitinating activity have different masses and/or charge; 2) each ubiquitin branched peptide can be made in a single solid phase synthesis by coupling a mixture of, for example, all 19 natural amino acids of different mass in a single coupling step at the X position in the synthesis; 3) each timecourse can be used as part of a series of assays (e.g., varying the concentration of the ubiquitin branched peptide) to calculate Km's for each substrate. Further, substrates with the smallest Km's (e.g., that bind with high affinity) can then selected and the isopeptide bond converted to a peptidomimetic bond, using methods known in the art and described herein, to make an uncleavable ubiquitin branched peptide that is an inhibitor of the deubiquitinating agent, and binds with high affinity to a deubiquitinating agent and thereby inhibits deubiquitinating activity of the deubiquitinating agent.

In another preferred embodiment, a branched ubiquitin peptide contains the C-terminus of a ubiquitin moiety ligated via an isopeptide bond to the lysine of a ubiquitin substrate (e.g., a target protein), and two or more amino acid residues from a ubiquitin substrate flanking the lysine attached to ubiquitin. Again, cleavage can be detected in a variety of ways, for example by observing the disappearance of the peptide absorbance peak representing the branched ubiquitin peptide, and appearance of two or more product peptides using reverse HPLC-mass spectrometry for detection. In a preferred embodiment the branched ubiquitin peptide minimally comprises: the ubiquitin moiety LRLRGG-, the branched lysine -K-, and the flanking amino acids (aa) 1-3 and 5-7 (e.g., (N-) aa1-aa2-aa3-K-aa5aa6-aa7-(-C)). Thus, the branched ubiquitin peptide minimally comprises the amino acid sequence, from amino to carboxl terminus, -aa1-aa2-aa3-K(GGRLRL)-aa5-aa6-aa7-, where flanking amino acids aa1-7 can be selected from the amino acid sequence flanking the lysine of a known ubiquitin substrate, where the lysine of the known ubiquitin substrate can be used for the attachment of a ubiquitin moiety. In another preferred embodiment, the branched ubiquitin peptide minimally comprises the amino acid sequence, from amino to carboxyl terminus KSSTY-(LRLRGG)-KTVA, where the lysine branch K and flanking amino acids are found in the histone H2B, a substrate for the deubiquitinating agent UbpM. In a preferred embodiment, the branched ubiquitin peptides are used in the methods of the present invention to assay candidate modulating agents for the ability to modulate deubiquitinating activity, e.g., block the cleavage or change the rate of cleavage of the ubiquitin branch peptide by a deubiquitinating agent. Such assays to not require the use of fluorophores or chromophores because the ubiquitin branched peptides and the peptide products of a deubiquitinating activity can be detected by absorption at a wave length of 206-220 nm.

In general, the components of the ubiquitin complex can be prepared by recombinant or synthetic methods as described herein or known in the art. For example, a ubiquitin moiety or ubiquitin substrate can be made by constructing and expressing a nucleic acid encoding such a polypeptide. Further, the ubiquitin complex of the present invention can be prepared using in vitro and in vivo ubiquitination reactions according to the methods as known in the art, described herein, or in e.g., U.S. Ser. No.10/091,174, filed Mar. 4, 2002; U.S. Ser. No. 10/091,139, filed Mar. 4, 2002; U.S. Ser. No.10/108,767, filed Mar. 26, 2002; U.S. Ser. No.10/109,460, filed Mar. 26, 2002; Weissman (2001) Nature Reviews 2:169-178, each expressly incorporated herein by reference. For example, branched ubiquitin peptide or ubiquitin fusion polypeptide can readily be synthesized or prepared by recombinant methods as in Dang et al. (1998) Biochemistry 37(7):1868-79.

In some preferred embodiments, the ubiquitin complex comprises a ubiquitin substrate. Such ubiquitin complexes can be formed using a variety of methods as described herein and known in the art. A preferred method for production of a ubiquitin complex in vitro is described in U.S. Ser. No. 10/091,174, filed Mar. 4, 2002; U.S. Ser. No.10/091,139, filed Mar. 4, 2002; U.S. Ser. No. 10/108,767, filed Mar. 26, 2002; U.S. Ser. No.10/109,460, filed Mar. 26, 2002, each of which is incorporated herein in their entirety. In a preferred embodiment, the ubiquitin complex in the methods of the present invention is formed by combining ubiquitin moiety and ubiquitin substrate in a reaction mixture in vitro or in a cell in vivo. In another preferred embodiment, the ubiquitin complex is purified and the purified ubiquitin complex combined in a reaction mixture in vitro with a deubiquitinating agent and assayed for deubiquitinating activity and, in a further aspect, the reaction mixture further comprises a candidate modulating agent and then assayed for the ability to modulate deubiquitinating activity.

In a preferred embodiment, the ubiquitin complex comprises a target protein comprising at least one ubiquitin moiety. In another preferred embodiment, the ubiquitin complex comprises a ubiquitin agent comprising at least one ubiquitin moiety. In another preferred embodiment, the ubiquitin agent is a ligating agent or a ubiquitin conjugating agent, as defined below. Thus, in general, ubiquitin agents are enzymes involved in ubiquitination.

In particular, the present invention provides ubiquitin agents that can be combined in different combinations with a ubiquitin moiety to form a ubiquitin complex where one or more ubiquitin moieties are attached to at least one of the following ubiquitin substrate molecules: a ubiquitin agent, a target protein, or a mono- or poly-ubiquitin moiety which is preferably attached to a ubiquitin agent or target protein.

In addition, the invention provides a variety of approaches for assaying for a candidate modulating agent that modulates the deubiquitinating activity of a deubiquitinating agent and, preferably, modulates the binding, release, or cleavage of a ubiquitin moiety from a ubiquitin complex. Examples of these approaches are as follows:

1. the components of the assay are combined in solution phase, and then assayed for modulation of deubiquitinating activity; or

2. the components of the assay are combined in solid phase by providing a ubiquitin complex or component of the ubiquitin complex (e.g., a target protein, ubiquitin agent, ubiquitin moiety, or cleavable ubiquitin fusion polypeptide) on a solid support, and then assayed for modulation of deubiquitinating activity; or

3. the components of the assay are combined in solution phase, thereafter a ubiquitin complex or component of a ubiquitin complex (e.g., a target protein, ubiquitin agent, ubiquitin moiety, or cleavable ubiquitin fusion polypeptide) is attached to a solid substrate, and then assayed for modulation of deubiquitinating activity; or

4. the components for forming the ubiquitin complex are combined in solution phase to form a ubiquitin complex, then a ubiquitin complex is purified, the purified ubiquitin complex or component of the purified ubiquitin complex (e.g., a target protein, ubiquitin agent, ubiquitin moiety, or cleavable ubiquitin fusion polypeptide) is then attached to a solid substrate and thereafter, a deubiquitinating agent is combined with the attached ubiquitin complex or component thereof, and assayed for modulation of deubiquitinating activity.

Examples of ubiquitin agents are ubiquitin activating agents, ubiquitin conjugating agents, and ubiquitin ligating agents.

As used herein “ubiquitin activating agent” refers to a ubiquitin agent, preferably a protein, capable of transferring or attaching a ubiquitin moiety to a ubiquitin conjugating agent. In a preferred embodiment, the ubiquitin activating agent forms a high energy thiolester bond with ubiquitin moiety, thereby “activating” the ubiquitin moiety. In another preferred embodiment, the ubiquitin activating agent binds or attaches ubiquitin moiety.

In a preferred embodiment the ubiquitin activating agent is an E1. In a preferred embodiment, the E1 is capable of transferring or attaching ubiquitin moiety to an E2, defined below.

Sequences encoding a ubiquitin activating agent may also be used to make variants thereof that are suitable for use in the methods and compositions of the present invention. The ubiquitin activating agents and variants suitable for use in the methods and compositions of the present invention can be prepared using the methods and sequences known in the art, described herein, or in e.g., U.S. Ser. No.10/091,174, filed Mar. 4, 2002; U.S. Ser. No.10/091,139, filed Mar. 4, 2002; U.S. Ser. No. 10/108,767, filed Mar. 26, 2002; U.S. Ser. No.10/109,460, filed Mar. 26, 2002; Weissman (2001) Nature Reviews 2:169-178, each expressly incorporated herein by reference.

In some embodiments, the methods of the present invention comprise the use of a ubiquitin conjugating agent. As used herein “ubiquitin conjugating agent” refers to a ubiquitin agent, preferably a protein, capable of transferring or attaching ubiquitin moiety to a ubiquitin ligating agent. In many cases, the ubiquitin conjugating agent is capable of directly transferring or attaching ubiquitin moiety to lysine residues in a target protein (Hershko et al. (1983) J. Biol. Chem. 258:8206-8214). In a preferred embodiment, the ubiquitin conjugating agent is capable of transferring or attaching ubiquitin moiety to a mono- or poly-ubiquitin moiety preferably attached to a ubiquitin agent or target protein. In a preferred embodiment, the ubiquitin conjugating agent is capable of transferring ubiquitin moiety to a mono- or poly-ubiquitinated ubiquitin ligating agent.

In a preferred embodiment the ubiquitin conjugating agent is an E2. In a preferred embodiment, ubiquitin moiety is transferred from E1 to E2. In a preferred embodiment, the transfer results in a thiolester bond formed between E2 and ubiquitin moiety. In a preferred embodiment, E2 is capable of transferring or attaching ubiquitin moiety to an E3, defined below.

Sequences encoding a ubiquitin conjugating agent may also be used to make variants thereof that are suitable for use in the methods and compositions of the present invention. The ubiquitin conjugating agents and variants suitable for use in the methods and compositions of the present invention can be prepared using the methods and sequences known in the art, described herein, or in e.g., U.S. Ser. No.10/091,174, filed Mar. 4, 2002; U.S. Ser. No.10/091,139, filed Mar. 4, 2002; U.S. Ser. No.10/108,767, filed Mar. 26, 2002; U.S. Ser. No.10/109,460, filed Mar. 26, 2002; Weissman (2001) Nature Reviews 2:169-178, each expressly incorporated herein by reference.

In a preferred embodiment, E2 has a tag, as defined below, with the complex being referred to herein as “tag-E2”. Preferred E2 tags include, but are not limited to, labels as defined below, partners of binding pairs and substrate binding elements. In a most preferred embodiment, the tag is a His-tag or GST-tag.

In some embodiments, the methods of the present invention comprise the use of a ubiquitin ligating agent. As used herein “ubiquitin ligating agent” refers to a ubiquitin agent, preferably a protein, capable of transferring or attaching a ubiquitin moiety to a target molecule or directing the transfer or attachment of a ubiquitin moiety from an E2 to a target molecule. In some cases, the ubiquitin agent is capable of transferring or attaching or directing the transfer or attachment of a ubiquitin moiety to itself or another ubiquitin ligating agent. In a preferred embodiment, the ubiquitin ligating agent is an E3.

As used herein “E3” refers to a ubiquitin ligating agent comprising one or more subunits, preferably polypeptides, associated with the activity of E3 as a ubiquitin ligating agent (i.e., associated with the ligation or attachment of ubiquitin moiety to a target protein, and in some cases, to itself or another E3). In a preferred embodiment, E3 is a member of the HECT domain E3 ligating agents. In another preferred embodiment, E3 is a member of the RING finger domain E3 ligating agents. In a preferred embodiment, E3 comprises a ring finger subunit and a Cullin subunit. Examples of RING finger polypeptides suitable for use in the methods and compositions of the present invention include, but are not limited to, ROC1, ROC2 and APC11. Examples of Cullin polypeptides suitable for use in the methods and compositions of the present invention include, but are not limited to, CUL1, CUL2, CUL3, CUL4A, CUL4B, CUL5 and APC2. In another preferred embodiment, the E3 is mdm2.

Sequences encoding a ubiquitin ligating agent may also be used to make variants thereof that are suitable for use in the methods and compositions of the present invention. The ubiquitin ligating agents and variants suitable for use in the methods and compositions of the present invention can be prepared using the methods and sequences known in the art, described herein, or in e.g., U.S. Ser. No.10/091,174, filed Mar. 4, 2002; U.S. Ser. No.10/091,139, filed Mar. 4, 2002; U.S. Ser. No. 10/108,767, filed Mar. 26, 2002; U.S. Ser. No.10/109,460, filed Mar. 26, 2002; Weissman (2001) Nature Reviews 2:169-178, each expressly incorporated herein by reference.

In a preferred embodiment, E3 comprises the RING finger protein/Cullin combination APC11/APC2. In another preferred embodiment, E3 comprises the RING finger protein/Cullin combination ROC1/CUL1. In yet preferred embodiment, E3 comprises the RING finger protein/Cullin combination ROC1/CUL2. In still another preferred embodiment, E3 comprises the RING finger protein/Cullin combination ROC2/CUL5. However, the skilled artisan will appreciate that any combination of E3 components may be produced and used in the invention described herein.

In a preferred embodiment, the E3 components are produced recombinantly, as described herein. In a preferred embodiment, the E3 components are co-expressed in the same host cell. Co-expression may be achieved by transforming the cell with a vector comprising nucleic acids encoding two or more of the E3 components, or by transforming the host cell with separate vectors, each comprising a single component of the desired E3 protein complex. In a preferred embodiment, the RING finger protein and Cullin are expressed in a single host transfected with two vectors, each comprising nucleic acid encoding one or the other polypeptide, as described in further detail in the Examples.

In a preferred embodiment, E3 has a tag, and this complex is referred to herein as “tag-E3”. Preferably, the tag is attached to only one component of the E3. Preferred E3 tags include, but are not limited to, labels, partners of binding pairs and substrate binding elements. More preferably, the tag is a surface substrate binding molecule. Most preferably, the tag is a His-tag or GST-tag.

In preferred embodiments, the ubiquitin activating agent is preferably an E1 or a variant thereof; the ubiquitin conjugating agent is preferably an E2 or a variant thereof; and the ubiquitin ligating agent is preferably an E3 or variant thereof. In a preferred embodiment, the E3 is Mdm2. In another preferred embodiment, the Mdm2 is a fusion protein, and more preferably a GST-Mdm2 fusion protein. Thus, in preferred embodiments, the ubiquitin complex comprises a ubiquitin moiety attached to a ubiquitin agent, target protein, or mono- or poly-ubiquitin moiety that is preferably attached to a ubiquitin agent or target protein. Alternatively, in another preferred embodiment, the ubiquitin complex comprises a cleavable ubiquitin fusion polypeptide comprising a ubiquitin moiety. For example, in a preferred embodiment, the ubiquitin fusion polypeptide is a branched ubiquitin peptide.

The ubiquitin complex comprising one or more ubiquitin or polyubiquitin moieties attached to a ubiquitin substrate can be formed by combining a ubiquitin moiety and one or more ubiquitin agents either in the presence of or in the absence of a target protein. The ubiquitin complex can be formed in cells in vivo or in a reaction mixture in vitro and used without purification in the methods of the present invention. In a preferred embodiment, the ubiquitin complex is purified for use in the methods of the present invention.

As used herein, “ubiquitin substrate molecule”, “ubiquitin substrate”, or “target substrate” and grammatical equivalents thereof means a molecule, preferably a protein, to which a ubiquitin moiety can be bound or attached by the activity of a ubiquitin agent or process of ubiquitination; or alternatively, by synthetic or recombinant means. As used herein with reference to the activity of ubiquitin agents, “attachment” refers to the transfer, binding, ligation, and/or ubiquitination of a mono- or poly-ubiquitin moiety to a ubiquitin substrate. Thus, “ubiquitination” and grammatical equivalents thereof means the attachment, or transfer, binding, and/or ligation of ubiquitin moiety to a ubiquitin substrate; and “ubiquitination reaction” and grammatical equivalents thereof refer to the combining of components under conditions that permit ubiquitination (i.e., the attachment or transfer, binding, and/or ligation of ubiquitin moiety to a substrate molecule).

In some preferred embodiments, the ubiquitin agent comprises a ubiquitin moiety. As used herein, the phrase “comprising a ubiquitin moiety” or grammatical equivalents thereof refers to a ubiquitin moiety fused, ligated, attached, or bound to another polypeptide. For example, the ubiquitin fusion polypeptide of the present invention comprises a ubiquitin moiety. Additionally, the phrase “comprising a ubiquitin moiety” or grammatical equivalents thereof, when used with reference to a ubiquitin agent, refers to the pre-loading, pre-conjugation, or pre-attachment of a ubiquitin moiety to a polypeptide, for example, a ubiquitin agent (forming a “pre-conjugated ubiquitin agent” or “pre-loaded ubiquitin agent”) such that the attachment of a ubiquitin moiety to a ubiquitin substrate does not require combining all three ubiquitin agents (i.e., a ubiquitin activating agent, ubiquitin conjugating agent, and ubiquitin ligating agent) and/or combining ubiquitin moiety that is not pre-conjugated. For example in the case of a ubiquitin activating agent comprising a ubiquitin moiety, the attachment of ubiquitin moiety to a ubiquitin conjugating agent can be performed in the absence of ubiquitin moiety that is not pre-conjugated. For example, in the case of a ubiquitin conjugating agent comprising a ubiquitin moiety, the attachment of ubiquitin moiety to a ubiquitin ligating agent can be performed in the absence of a ubiquitin activating agent and ubiquitin moiety that is not pre-conjugated. Also, for example, in the case of a ubiquitin ligating agent comprising a ubiquitin moiety, the attachment of ubiquitin moiety to a target molecule can be performed in the absence of a ubiquitin activating agent, ubiquitin conjugating agent, and ubiquitin moiety that is not pre-conjugated. A pre-conjugated ubiquitin agent suitable for use in the methods and compositions of the present invention can be prepared using methods described herein. In a preferred embodiment, pre-conjugated ubiquitin agents are prepared according to Zhihong et al. (2001) J.Biol.Chem. 276:31,357-31,367.

By “target protein” herein is meant a protein other than a ubiquitin moiety to which a ubiquitin moiety is bound or attached through the activity of a ubiquitin agent or by the process of ubiquitination. In preferred embodiments, the target protein is a mammalian target protein, and more preferably a human target protein. In a preferred embodiment, the target protein is p53.

In a preferred embodiment of the methods for assaying for a candidate modulating agent, the combining further comprises combining a ubiquitin activating agent, ubiquitin conjugating agent, ubiquitin ligating agent, and the target protein, and thereby forming the target protein comprising at least one ubiquitin moiety.

In a preferred embodiment of the methods for assaying for a candidate modulating agent, the combining further comprises combining a ubiquitin activating agent comprising the ubiquitin moiety, ubiquitin conjugating agent, and the target protein, and thereby forming the target protein comprising at least one ubiquitin moiety.

In a preferred embodiment of the methods for assaying for a candidate modulating agent, the combining further comprises combining a ubiquitin conjugating agent comprising the ubiquitin moiety and the target protein, and thereby forming the target protein comprising at least one ubiquitin moiety.

In a preferred embodiment, a ubiquitin complex (e.g., a ubiquitin fusion polypeptide) or a component of the ubiquitin complex (e.g., a ubiquitin substrate) is attached to the surface of a reaction vessel, such as the well of a multi-well plate, or other type of solid support, e.g., a bead, including fluroscent or magnetic beads. This embodiment facilitates separation and detection of the products of a deubiquitinating reaction, e.g., the intact ubiquitin complex, cleaved ubiquitin complex, and released, cleaved, or free ubiquitin moiety. Means for attaching a ubiquitin complex or components of a ubiquitin complex to the surface of a reaction vessel are described below. The present methods permits the entire assay to occur in one vessel, making the assay useful for high-throughput screening applications.

In a preferred embodiment, the ubiquitin complex or a component of the ubiquitin complex, and preferably the ubiquitin moiety of the ubiquitin complex is labeled, either directly or indirectly, as further described below, and the amount of label is measured and indicative of the presence and/or amount of deubiquitinating activity. Thus, the invention provides methods that permit the easy and rapid detection and measurement of deubiquitinating activity, making the assay useful for high-throughput screening applications. In one preferred embodiment, the signal of the label varies with the extent of the cleavage or release of ubiquitin moiety from the ubiquitin complex, such as in the FRET system described below (see also, e.g., the FRET system of Boisclair et al. (2000) J. Biomol. Screen 5(5):319-328, incorporated herein by reference). One of ordinary skill in the art will recognize the applicability of the present invention to screening for agents which modulate deubiquitinating activity or to screen for deubiquitinating agents.

As used herein, “ubiquitin moiety” refers to polypeptide compromising a biologically active domain of a ubiquitin polypeptide or ubiquitin-like molecule, for example, a domain associated with an activity or function of ubiquitin (e.g., is recognized, bound, transferred, or ligated by a ubiquitin agent; or is recognized, bound, released, or cleaved by a deubiquitinating agent). In preferred embodiments, the ubiquitin moiety comprises: 1) a full-length ubiquitin polypeptide, or biologically active domain thereof, fused to another ubiquitin or to another polypeptide (e.g., a ubiquitin fusion polypeptide, particularly, a cleavable ubiquitin fusion polypeptide or a branched ubiquitin peptide); or 2) which is transferred or attached to another polypeptide (e.g., a ubiquitin substrate) by a ubiquitin agent. The ubiquitin moiety can comprise a ubiquitin from any species of organism, preferably a eukaryotic species. In preferred embodiments the ubiquitin moiety comprises is a mammalian ubiquitin, and more preferably a human ubiquitin. In a preferred embodiment, the ubiquitin moiety comprises a 76 amino acid human ubiquitin. Other embodiments utilize variants and derivative of ubiquitin, as further described below. As used herein, “poly-ubiquitin moiety” or grammatical equivalents thereof refers to a chain of ubiquitin moieties comprising more than one ubiquitin moiety. As used herein, “mono-ubiquitin moiety” or grammatical equivalents thereof refers to a single ubiquitin moiety. In the methods of the present invention, a mono- or poly-ubiquitin moiety can serve as a substrate molecule for the transfer or attachment of ubiquitin moiety (which can itself be a mono- or poly-ubiquitin moiety).

In a preferred embodiment, when ubiquitin moiety is attached to a target protein, that protein is targeted for degradation by the 26S proteasome.

As used herein, “ubiquitin moiety” encompasses naturally occurring alleles and man-made variants of such a 76 amino acid polypeptide. Ubiquitin moiety also includes variants of ubiquitin-like molecules. The ubiquitin moiety and variants suitable for use in the methods and compositions of the present invention can be prepared using the methods and sequences known in the art, described herein, or in e.g., U.S. Ser. No.10/091,174, filed Mar. 4, 2002; U.S. Ser. No.10/091,139, filed Mar. 4, 2002; U.S. Ser. No.10/108,767, filed Mar. 26, 2002; U.S. Ser. No.10/109,460, filed Mar. 26, 2002; Weissman (2001) Nature Reviews 2:169-178, each expressly incorporated herein by reference.

In a preferred embodiment, the ubiquitin moiety comprises an amino acid sequence or nucleic acid sequence corresponding to a sequence of GENBANK accession number P02248, incorporated herein by reference. In other preferred embodiments, the ubiquitin moiety comprises an amino acid sequence or nucleic acid sequence of a sequence corresponding to one of the following GENBANK accession numbers: NM_{—}006156 (NEDD8); NM_{—}003352 (SUMO-1, aka, UBL1); XM_{—}048691 (SUMO-1, aka, UBL1); NM_{—}006936 (smt3a); XM_{—}009805 (smt3a); XM_{—}095400 (smt3b); NM_{—}006937 (smt3b); XM_{—}041583 (smt3b); NM_{—}015783 (ISG15); or NM_{—}005101 (ISG15), each incorporated herein by reference.

As described below, GENBANK accession numbers and their corresponding amino acid sequences or nucleic acid sequences are found in the GenBank data base. Sequences corresponding to GenBank accession numbers cited herein are incorporated herein by reference. GenBank is known in the art, see, e.g., Benson, D A, etal., Nucleic Acids Research 26:1-7 (1998) and www.ncbi.nlm.nih.gov. In a preferred embodiment, variants of a particular ubiquitin moiety have an overall amino acid sequence identity of preferably greater than about 75%, more preferably greater than about 80%, even more preferably greater than about 85% and most preferably greater than 90% of the amino acid sequence of the particular ubiquitin moiety. In some embodiments the sequence identity will be as high as about 93 to 95 or 98%.

In another preferred embodiment, variants of a particular ubiquitin moiety have an overall sequence similarity with the amino acid sequence of the particular ubiquitin moiety of greater than about 80%, more preferably greater than about 85%, even more preferably greater than about 90% and most preferably greater than 93%. In some embodiments the sequence identity will be as high as about 95 to 98 or 99%.

As is known in the art, a number of different programs can be used to identify whether a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http://blast.wustl/edu/blast/README.html]. WU-BLAST-2 uses several search parameters, most of which are set to the default values. The adjustable parameters are set with the following values: overlap span ′1, overlap fraction ′0.125, word threshold (T) ′11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25:3389-3402. Gapped BLAST uses BLOSUM-62 substitution scores; threshold T parameter set to 9; the two-hit method to trigger ungapped extensions; charges gap lengths of k a cost of 10+k; Xᵤ set to 16, and X_{g} set to 40 for database search stage and to 67 for the output stage of the algorithms. Gapped alignments are triggered by a score corresponding to ˜22 bits.

A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment may include the introduction of gaps in the sequences to be aligned. In addition, for sequences which contain either more or fewer amino acids than a particular amino acid sequence of interest, it is understood that in one embodiment, the percentage of sequence identity will be determined based on the number of identical amino acids in relation to the total number of amino acids. Thus, for example, sequence identity of sequences shorter than that of a sequence of interest, as discussed below, will be determined using the number of amino acids in the shorter sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

Ubiquitin moieties of the present invention are polypeptides that may be shorter or longer than a full-length ubiquitin protein. In one embodiment herein, fragments of ubiquitin moiety are considered ubiquitin moieties if they are attached to another polypeptide by a ubiquitin agent.

In addition, as is more fully outlined below, ubiquitin moieties of the present invention are polypeptides that can be made longer than the amino acid sequence encoding a ubiquitin amino acid sequence; for example, by the addition of tags, the addition of other fusion sequences, or the elucidation of additional coding and non-coding sequences. As described below, the fusion of a ubiquitin moiety to a fluorescent peptide, such as Green Fluorescent Peptide (GFP), is particularly preferred.

The ubiquitin moiety, as well as other proteins of the present invention, are preferably recombinant proteins. A “recombinant protein” is a protein made using recombinant techniques, i.e. through the expression of a recombinant nucleic acid as described below. In a preferred embodiment, the ubiquitin moiety of the invention is made through the expression of a nucleic acid sequence corresponding to GENBANK accession number M26880 or AB003730, or a fragment thereof. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

As used herein and further defined below, “nucleic acid” may refer to either DNA or RNA, or molecules which contain both deoxy- and ribonucleotides. The nucleic acids include genomic DNA, cDNA and oligonucleotides including sense and anti-sense nucleic acids. Such nucleic acids may also contain modifications in the ribose-phosphate backbone to increase stability and half life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”); thus the sequences depicted herein also include the complement of the sequence.

By the term “recombinant nucleic acid” herein is meant nucleic acid, originally formed in vitro, in general, by the manipulation of nucleic acid by endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

The terms “polypeptide” and “protein” may be used interchangeably throughout this application and mean at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradation.

In one embodiment, the present invention provides compositions containing protein variants, for example, variants of deubiquitinating agents, ubiquitin moieties, ubiquitin agents, e.g., E1, E2 and E3. These variants fall into one or more of three classes: substitutional, insertional or deletional variants. These variants ordinarily are prepared by site specific mutagenesis of nucleotides in the DNA encoding a protein of the present compositions, using cassette or PCR mutagenesis or other techniques well known in the art, to produce DNA encoding the variant, and thereafter expressing the DNA in recombinant cell culture as outlined above. However, variant protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed variants screened for the optimal desired activity. Techniques for making substitution mutations at predetermined sites in DNA having a known sequence are well known, for example, M13 primer mutagenesis and PCR mutagenesis. Rapid production of many variants may be done using techniques such as the method of gene shuffling, whereby fragments of similar variants of a nucleotide sequence are allowed to recombine to produce new variant combinations. Examples of such techniques are found in U.S. Pat. Nos. 5,605,703; 5,811,238; 5,873,458; 5,830,696; 5,939,250; 5,763,239; 5,965,408; and 5,945,325, each incorporated by reference herein in its entirety. Screening of the mutants is performed using the activity assays of the present invention.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the protein are desired, substitutions of an original residue are generally made in accordance with exemplary substitutions listed below.

| Original | Exemplary |
|---|---|
| Residue | Substitutions |
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in the above list. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the proteins as needed. Alternatively, the variant may be designed such that the biological activity of the protein is altered. For example, glycosylation sites may be altered or removed.

Covalent modifications of polypeptides are included within the scope of this invention. One type of covalent modification includes reacting targeted amino acid residues of a polypeptide with an organic derivatizing agent that is capable of reacting with selected side chains or the N-or C-terminal residues of a polypeptide. Derivatization with bifunctional agents is useful, for instance, for crosslinking a protein to a water-insoluble support matrix or surface for use in the method for screening assays, as is more fully described below. Commonly used crosslinking agents include, e.g., 1,1-bis(diazoacetyl)-2phenylethane, glutaraldehyde, Bhydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the □-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of a polypeptide included within the scope of this invention comprises altering the native glycosylation pattern of the polypeptide. “Altering the native glycosylation pattern” is intended for purposes herein to mean deleting one or more carbohydrate moieties found in native sequence polypeptide, and/or adding one or more glycosylation sites that are not present in the native sequence polypeptide.

Addition of glycosylation sites to polypeptides may be accomplished by altering the amino acid sequence thereof. The alteration may be made, for example, by the addition of, or substitution by, one or more serine or threonine residues to the native sequence polypeptide (for O-linked glycosylation sites). The amino acid sequence may optionally be altered through changes at the DNA level, particularly by mutating the DNA encoding the polypeptide at preselected bases such that codons are generated that will translate into the desired amino acids.

Another means of increasing the number of carbohydrate moieties on a polypeptide is by chemical or enzymatic coupling of glycosides to the polypeptide. Such methods are described in the art, e.g., in WO 87/05330 published Sep. 11, 1987, and in Aplin and Wriston, CRC Crit. Rev. Biochem., pp. 259-306 (1981).

Removal of carbohydrate moieties present on the polypeptide may be accomplished chemically or enzymatically or by mutational substitution of codons encoding for amino acid residues that serve as targets for glycosylation. Chemical deglycosylation techniques are known in the art and described, for instance, by Hakimuddin, etal., Arch. Biochem. Biophys., 259:52 (1987) and by Edge etal., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of a protein comprises linking the polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

Polypeptides of the present invention may also be modified in a way to form chimeric molecules comprising a first polypeptide fused to another, heterologous polypeptide or amino acid sequence. In a preferred embodiment, such a chimeric molecule is a ubiquitin fusion polypeptide, and more preferably a cleavable ubiquitin fusion polypeptide, comprising a ubiquitin moiety and another polypeptide. In a preferred embodiment, the chimeric polypeptide comprises a tag polypeptide which provides an epitope to which an anti-tag antibody can selectively bind. Also, in another embodiment, the chimeric molecule comprises a fusion of a ubiquitin substrate molecule (e.g., a ubiquitin moiety, ubiquitin agent, or target protein) with such a tag polypeptide. The epitope tag is generally placed at the amino-or carboxyl-terminus of the polypeptide. The presence of such epitope-tagged forms of a polypeptide can be detected using an antibody against the tag polypeptide. Also, providing an epitope tag enables the polypeptide to be readily purified by affinity purification using an anti-tag antibody or another type of affinity matrix that binds to the epitope tag. In an alternative embodiment, the chimeric molecule may comprise a fusion of a polypeptide disclosed herein with an immunoglobulin or a particular region of an immunoglobulin. For a bivalent form of the chimeric molecule, such a fusion could be to the Fc region of an IgG molecule. Tags for components of the invention are defined and described in detail below.

By “ubiquitin activating activity”, “ubiquitin moiety activation” and grammatical equivalents thereof is meant the binding or attachment of ubiquitin moiety to a substrate molecule that is preferably a ubiquitin activating agent. In a preferred embodiment, the ubiquitin activating agent is an E1. Preferably, the E1 forms a high energy thiolester bond with the ubiquitin moiety.

By “ubiquitin conjugating activity”, “ubiquitin moiety conjugation” and grammatical equivalents thereof is meant the binding or attachment of an activated ubiquitin moiety to a ubiquitin conjugating agent. As will be appreciated by those in the art, due to the presence of the high energy thiolester bond in the conjugate of the ubiquitin moiety-ubiquitin conjugating agent, the attached ubiquitin moiety may be joined to other ubiquitin moiety at a low rate in the absence of the catalytic activity of a ubiquitin ligating agent (e.g., E3). Therefore, some of the ubiquitin moiety can be attached in the form of polyubiquitin moiety.

By “ubiquitin ligating activity”, “ubiquitin moiety ligation” and grammatical equivalents thereof is meant the transfer or attachment of ubiquitin moiety to a substrate molecule that is preferably a target protein or mono- or poly-ubiquitin moiety preferably attached to a target protein. Preferably, each ubiquitin moiety is covalently attached by the ubiquitin ligating agent such that a subsequent ubiquitin moiety may be attached to it, to form chains (poly-ubiquitin moieties) comprising a plurality of ubiquitin moiety molecules.

By “combining” is meant the combining of the various components in a reaction mixture in vitro or in a cell in vivo under conditions in which the deubiquitination of a ubiquitin complex can occur (and optionally, formation of a ubiquitin complex). In some embodiments, the combining further comprises combining ubiquitin moiety and ubiquitin substrate to form a ubiquitin complex. In a preferred embodiment, the reaction mixture or cells are contained in a well of a 96 well plate or other commercially available multiwell plate. In an alternate preferred embodiment, the reaction mixture or cells are in a FACS machine. Other multiwell plates useful in the present invention include, but are not limited to 384 well plates and 1536 well plates. Still other vessels for containing the reaction mixture or cells and useful in the present invention will be apparent to the skilled artisan.

The addition of the components of the assay for deubiquitinating activity, or modulation of such activity, may be sequential or in a predetermined order or grouping under conditions appropriate for the activity that is assayed for, e.g. under conditions suitable for deubiquination. Such conditions are described here and known in the art,. Moreover, further guidance is provided below.

In a preferred embodiment, one or more components of the methods of the present invention comprise a tag. By “tag” is meant an attached molecule or molecules useful for the identification or isolation of the attached molecule(s), which are preferably substrate molecules. For example, a tag can be an attachment tag or a label tag. Components having a tag are referred to as “tag-X”, wherein X is the component. For example, a ubiquitin moiety comprising a tag is referred to herein as “tag-ubiquitin moiety” or a ubiquitin fusion polypeptide comprising a tag is referred to herein as “tag-ubiquitin fusion polypeptide”. Similarly, a cleavable ubiquitin fusion polypeptide comprising a tag is referred to herein as a “tag-cleavable ubiquitin fusion polypeptide”. Preferably, the tag is covalently bound to the attached component. When more than one component of a combination has a tag, the tags will be numbered for identification, for example “tag1-ubiquitin moiety”. Components may comprise more than one tag, in which case each tag will be numbered, for example “tag 1,2-ubiquitin moiety”. Preferred tags include, but are not limited to, a label, a partner of a binding pair, and a surface substrate binding molecule (or attachment tag). As will be evident to the skilled artisan, many molecules may find use as more than one type of tag, depending upon how the tag is used.

By “label” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is performed will depend on the label. Preferred labels include, but are not limited to, fluorescent labels, label enzymes and radioisotopes.

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705 and Oregon green. Suitable optical dyes are described in the 1996 Molecular Probes Handbook by Richard P. Haugland, hereby expressly incorporated by reference. Suitable fluorescent labels also include, but are not limited to, green fluorescent protein (GFP; Chalfie, et al, Science 263(5148):802-805 (Feb. 11, 1994); and EGFP; Clontech-Genbank Accession Number U55762 ), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), -galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558), and Ptilosarcus green fluorescent proteins (pGFP) (see WO 99/49019). All of the above-cited references are expressly incorporated herein by reference.

In a preferred embodiment, the fluorescent label is preferably a GFP and, more preferably, a renilla, ptilosarcus, or aequorea species of GFP (see e.g., U.S. Ser. No. 10/133,973, Filed Apr. 24, 2002, expressly incorporated herein by reference).

In some instances, multiple fluorescent labels are employed. In a preferred embodiment, at least two fluorescent labels are used which are members of a fluorescence resonance energy transfer (FRET) pair. In another preferred embodiment, the ubiquitin complex comprises a first and a second ubiquitin moiety, wherein the first and second ubiquitin moieties comprise different fluorescent labels, and wherein the labels form a FRET pair. Also, in another preferred embodiment, the first ubiquitin moiety comprises a FRET label and the second ubiquitin moiety comprises a quencher as further described below.

FRET is phenomenon known in the art wherein excitation of one fluorescent dye is transferred to another without emission of a photon. A FRET pair consists of a donor fluorophore and an acceptor fluorophore. The fluorescence emission spectrum of the donor and the fluorescence absorption spectrum of the acceptor must overlap, and the two molecules must be in close proximity. The distance between donor and acceptor at which 50% of donors are deactivated (transfer energy to the acceptor) is defined by the Förster radius (R₀), which is typically 10-100 Å. Changes in the fluorescence emission spectrum comprising FRET pairs can be detected, indicating changes in the number of that are in close proximity (i.e., within 100 Å of each other). This will typically result from the binding or dissociation of two molecules, one of which is labeled with a FRET donor and the other of which is labeled with a FRET acceptor, wherein such binding brings the FRET pair in close proximity. Binding of such molecules will result in an increased fluorescence emission of the acceptor and/or quenching of the fluorescence emission of the donor.

FRET pairs (donor/acceptor) useful in the invention include, but are not limited to, EDANS/fluorescien, IAEDANS/fluorescein, fluorescein/tetramethylrhodamine, fluorescein/LC Red 640, fluorescein/Cy 5, fluorescein/Cy 5.5 and fluorescein/LC Red 705.

In another aspect of FRET, a fluorescent donor molecule and a nonfluorescent acceptor molecule (“quencher”) may be employed. In this application, fluorescent emission of the donor will increase when quencher is displaced from close proximity to the donor and fluorescent emission will decrease when the quencher is brought into close proximity to the donor. Useful quenchers include, but are not limited to, TAMRA, DABCYL, QSY 7 and QSY 33. Useful fluorescent donor/quencher pairs include, but are not limited to EDANS/DABCYL, Texas Red/DABCYL, BODIPY/DABCYL, Lucifer yellow/DABCYL, coumarin/DABCYL and fluorescein/QSY 7 dye.

In a preferred embodiment, the ubiquitin complex comprises a poly-ubiquitin chain, and the polyubiquitin chain comprises at least two ubiquitin moieties. In another preferred embodiment, the ubiquitin complex comprises a poly-ubiquitin chain, and the poly-ubiquitin chain comprises a first ubiquitin moiety and a second ubiquitin moiety. In another preferred embodiment, the first ubiquitin moiety comprises a first label and the second ubiquitin moiety comprises a second label. Also in another preferred embodiment, the first ubiquitin moiety comprises a first FRET label and the second ubiquitin moiety comprises a second FRET label. In a further aspect, the first ubiquitin moiety comprises a FRET label and the second ubiquitin moiety comprises a Quencher.

In another preferred embodiment, the ubiquitin complex comprises the fluorogenic complex Ubiquitin-AMC (7-amido-4-methylcoumarin), commercially available from Calbiochem (Cat. No. 662075) and Boston Biochem (Cat. No. U-550).

The skilled artisan will appreciate that FRET and fluorescence quenching allow for monitoring of binding of labeled molecules over time, providing continuous information regarding the time course of binding reactions.

In a preferred embodiment, the ubiquitin complex comprises a ubiquitin moiety attached to a ubiquitin substrate molecule, and the terminal carboxyl group of the ubiquitin moiety is ligated to a lysine residue of a ubiquitin substrate molecule. Therefore, attachment of labels or other tags should not interfere with either of these active groups on the ubiquitin moiety. Amino acids may be added to the sequence of protein, through means well known in the art and described herein, for the express purpose of providing a point of attachment for a label. In a preferred embodiment, one or more amino acids are added to the sequence of a component for attaching a tag thereto, preferably a fluorescent label. In a preferred embodiment, the amino acid to which a fluorescent label is attached is Cysteine.

By “label enzyme” is meant an enzyme which may be reacted in the presence of a label enzyme substrate which produces a detectable product. Suitable label enzymes for use in the present invention include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J Virol. Methods 24:227-236 (1989), which are each hereby incorporated by reference in their entirety.

By “radioisotope” is meant any radioactive molecule. Suitable radioisotopes for use in the invention include, but are not limited to ¹⁴C, ³H, ³²P, ³³P, ³⁵S, ¹²⁵I, and ¹³¹I. The use of radioisotopes as labels is well known in the art.

In addition, labels may be indirectly detected, that is, the tag is a partner of a binding pair. By “partner of a binding pair” is meant one of a first and a second moiety, wherein the first and the second moiety have a specific binding affinity for each other. Suitable binding pairs for use in the invention include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avid (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin etal., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto. Generally, in a preferred embodiment, the smaller of the binding pair partners serves as the tag, as steric considerations in ubiquitin moiety ligation may be important. As will be appreciated by those in the art, binding pair partners may be used in applications other than for labeling, as is further described below.

As will be appreciated by those in the art, a partner of one binding pair may also be a partner of another binding pair. For example, an antigen (first moiety) may bind to a first antibody (second moiety) which may, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair may comprise a label, as described above. It will further be appreciated that this allows for a tag to be indirectly labeled upon the binding of a binding partner comprising a label. Attaching a label to a tag which is a partner of a binding pair, as just described, is referred to herein as “indirect labeling”.

By “surface substrate binding molecule” or “attachment tag” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate [Field et al., Mol. Cell. Biol., 8:2159-2165 (1988)]; the c-myc tag and the 8F9, 3C7, 6E10, G4, B7 and 9E10 antibody substrates thereto [Evan et al., Molecular and Cellular Biology, 5:3610-3616 (1985)]; and the Herpes Simplex virus glycoprotein D (gD) tag and its antibody substrate [Paborsky et al., Protein Engineering, 3(6):547-553 (1990)]. In general, surface binding substrate molecules useful in the present invention include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

Production of antibody-embedded substrates is well known; see Slinkin et al., Bioconj. Chem. 2:342-348 (1991); Torchilin et al., supra; Trubetskoy et al., Bioconj. Chem. 3:323-327 (1992); King et al, Cancer Res. 54:6176-6185 (1994); and Wilbur et al., Bioconjucate Chem. 5:220-235 (1994) (all of which are hereby expressly incorporated by reference), and attachment of or production of proteins with antigens is described above.

Calmodulin-embedded substrates are commercially available , and production of proteins with CBP is described in Simcox et al., Strategies 8:40-43 (1995), which is hereby incorporated by reference in its entirety.

As will be appreciated by those in the art, tag-components of the invention can be made in various ways, depending largely upon the form of the tag. Components of the invention and tags are preferably attached by a covalent bond.

The production of tag-polypeptides by recombinant means when the tag is also a polypeptide is described below. Production of tag-labeled proteins is well known in the art and kits for such production are commercially available (for example, from Kodak and Sigma). Examples of tag labeled proteins include, but are not limited to, a Flag-polypeptide and His-polypeptide. Methods for the production and use of tag-labeled proteins are found, for example, in Winston et al., Genes and Devel. 13:270-283 (1999), incorporated herein in its entirety, as well as product handbooks provided with the above-mentioned kits.

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known (Id.).

Methods for labeling of proteins with radioisotopes are known in the art. For example, such methods are found in Ohta et al., Molec. Cell 3:535-541 (1999), which is hereby incorporated by reference in its entirety.

Production of proteins having tags by recombinant means is well known, and kits for producing such proteins are commercially available. For example, such a kit and its use is described in the QIAexpress Handbook from Qiagen by Joanne Crowe et al., hereby expressly incorporated by reference.

The functionalization of labels with chemically reactive groups such as thiols, amines, carboxyls, etc. is generally known in the art. In a preferred embodiment, the tag is functionalized to facilitate covalent attachment. In a preferred embodiment, the tag is a His tag, Flag tag, or GST tag.

The covalent attachment of the tag may be either direct or via a linker. In one embodiment, the linker is a relatively short coupling moiety, that is used to attach the molecules. A coupling moiety may be synthesized directly onto a component of the invention, e.g., a ubiquitin moiety, and contains at least one functional group to facilitate attachment of the tag. Alternatively, the coupling moiety may have at least two functional groups, which are used to attach a functionalized component to a functionalized tag, for example. In an additional embodiment, the linker is a polymer. In this embodiment, covalent attachment is accomplished either directly, or through the use of coupling moieties from the component or tag to the polymer. In a preferred embodiment, the covalent attachment is direct, that is, no linker is used. In this embodiment, the component preferably contains a functional group such as a carboxylic acid which is used for direct attachment to the functionalized tag. It should be understood that the component and tag may be attached in a variety of ways, including those listed above. What is important is that the manner of attachment does not significantly alter the functionality of the component, for example the manner of attachment should not alter the ability to fuse or attach a ubiquitin moiety to another ubiquitin or another polypeptide where the fusion or attachment of the ubiquitin moiety to another ubiquitin or another polypeptide is desired. In a preferred embodiment, the tag is attached to the amino or carboxl terminus of the polypeptide. For example, in a preferred embodiment, the ubiquitin fusion polypeptide of the present invention comprises a tag at the amino terminus and/or carboxyl terminus. As will be appreciated by those in the art, the above description of covalent attachment of a label and ubiquitin moiety applies equally to the attachment of virtually any two molecules of the present disclosure.

In a preferred embodiment, the tag is functionalized to facilitate covalent attachment, as is generally outlined above. Thus, a wide variety of tags are commercially available which contain functional groups, including, but not limited to, isothiocyanate groups, amino groups, haloacetyl groups, maleimides, succinimidyl esters, and sulfonyl halides, all of which may be used to covalently attach the tag to a second molecule, as is described herein. The choice of the functional group of the tag will depend on the site of attachment to either a linker, as outlined above or a component of the invention. Thus, for example, for direct linkage to a carboxylic acid group of a ubiquitin moiety, amino modified or hydrazine modified tags will be used for coupling via carbodiimide chemistry, for example using 1-ethyl-3-(3-dimethylaminopropyl)-carbodiimide (EDAC) as is known in the art (see Set 9 and Set 11 of the Molecular Probes Catalog, supra; see also the Pierce 1994 Catalog and Handbook, pages T-155 to T-200, both of which are hereby incorporated by reference). In one embodiment, the carbodiimide is first attached to the tag, such as is commercially available for many of the tags described herein.

In a preferred embodiment, ubiquitin moiety is in the form of tag-ubiquitin moiety, wherein, tag is a partner of a binding pair. Preferably in this embodiment the tag is FLAG and the binding partner is anti-FLAG. Preferably in this embodiment, a label is attached to the FLAG by indirect labeling. Preferably, the label is a label enzyme. Most preferably, the label enzyme is horseradish peroxidase, which is reacted with a fluorescent label enzyme substrate. Preferably, the label enzyme substrate is Luminol. Alternatively, the label is a fluorescent label.

In another preferred embodiment, ubiquitin moiety is in the form of tag-ubiquitin moiety, wherein the tag is a fluorescent label. In a particularly preferred embodiment, ubiquitin moiety is in the form of tag1-ubiquitin moiety and tag2-ubiquitin moiety, wherein tag1 and tag2 are the members of a FRET pair. In an alternate preferred embodiment, ubiquitin moiety is in the form of tag1-ubiquitin moiety and tag2-ubiquitin moiety, wherein tag 1 is a fluorescent label and tag2 is a quencher of the fluorescent label. In either of these preferred embodiments, when tag1-ubiquitin moiety and tag2-ubiquitin moiety are attached to a substrate molecule of interest through the activity of a ubiquitin agent, preferably tag1 and tag2 are within 100 Angstroms of each other, more preferable within 70 Angstroms, still more preferably within 50 Angstroms, even more preferably within 40 Angstroms, and in some cases, preferably within 30 Angstroms or less.

In yet another preferred embodiment, ubiquitin moiety is in the form of tag1, 2-ubiquitin moiety and tag1,3-ubiquitin moiety, wherein tag1 is a member of a binding pair, preferably FLAG, tag2 is a fluorescent label and tag3 is either a fluorescent label such that tag2 and tag3 are members of a FRET pair or tag3 is a quencher of tag2.

In a preferred embodiment, one or more amino acids are added to the ubiquitin moiety sequence, using recombinant techniques as described herein, to provide an attachment point for a tag, preferably a fluorescent label or a quencher. In a preferred embodiment, the one or more amino acids are Cys or Ala-Cys. Preferably, the one or more amino acids are attached to the N-terminal of the ubiquitin moiety. In a preferred embodiment, the one or more amino acids intervenes the sequence of a FLAG tag and the ubiquitin moiety. In a preferred embodiment, the tag, preferably a fluorescent label or a quencher, is attached to the added Cysteine.

As used herein, “deubiquitinating agent” encompasses naturally occurring alleles and man-made variants of a deubiquitinating enzyme. In a preferred embodiment, the deubiquitinating agent comprises an amino acid sequence or a nucleic acid sequence of a sequence corresponding to an accession number in the GenBank data base or ENSEMBL data base (a joint project of the European Molecular Biology Laboratories and the Sanger Institute) listed in Table 1 below and incorporated herein by reference. The accession numbers from the GenBank data base at www.ncbi.nlm.nih.gov. The accession numbers from the ENSEMBL data base are found at www.ensembl.org.

TABLE 1

| | |
|---|---|
| nucleic acid | amino acid |

GenBank Accession No.s

| | |
|---|---|
| XM_086378 | XP_086378 |
| XM_088736 | XP_088736 |
| NM_024292 | NP_077268 |
| M10939 | AAA36788 |
| NM_007278 | NP_009209 |
| XM_086494 | XP_086494 |
| NM_007285 | NP_009216 |
| NM_014235 | NP_055050 |
| BC012472 | AAH12472 |
| AF251700 | AAL99389 |
| XM_063384 | XP_063384 |
| XM_064899 | XP_064899 |
| BC008450 | AAH08450 |
| XM_030786 | XP_030786 |
| BC019910 | AAH19910 |
| BC014367 | AAH14367 |
| NM_032514 | NP_115903 |
| NM_001997 | NP_001988 |
| XM_087907 | XP_087907 |
| AK026593 | BAB15505 |
| XM_092407 | XP_092407 |
| XM_113737 | XP_113737 |
| AF348700 | AAK31162 |
| AF077046 | AAD27779 |
| NM_003333 | NP_003324 |
| XM_089415 | XP_089415 |
| NM_006156 | NP_006147 |
| XM_114058 | XP_114058 |
| XM_168354 | XP_168354 |
| NM_004707 | NP_004698 |
| NM_007106 | NP_009037 |
| NM_007108 | NP_009039 |
| NM_032568 | NP_115957 |
| NM_002954 | NP_002945 |
| NM_003352 | NP_003343 |
| NM_005101 | NP_005092 |
| NM_006936 | NP_008867 |
| XM_009805 | XP_009805 |
| XM_115124 | XP_115124 |
| BC011033 | AAH11033 |
| NM_024571 | NP_078847 |
| XM_093349 | XP_093349 |
| XM_091851 | XP_091851 |
| XM_166749 | XP_166749 |
| NM_022818 | NP_073729 |
| XM_058745 | XP_058745 |
| XM_066029 | XP_066029 |
| NM_006398 | NP_006389 |
| NM_003363 | NP_003354 |
| NM_006313 | NP_006304 |
| AF383173 | AAL78315 |
| AF130096 | AAG35521 |
| AB029020 | BAA83049 |
| BC003130 | AAH03130 |
| XM_113421 | XP_113421 |
| NM_004654 | NP_004645 |
| XM_166244 | XP_166244 |
| XM_070195 | XP_070195 |
| XM_167111 | XP_167111 |
| NM_003470 | NP_003461 |
| XM_065679 | XP_065679 |
| XM_093206 | XP_093206 |
| AF353989 | AAK49524 |
| AF217979 | AAG17222 |
| NM_014871 | NP_055686 |
| AK001647 | BAA91807 |
| BC016146 | AAH16146 |
| NM_020903 | NP_065954 |
| BC013737 | AAH13737 |
| AB046814 | BAB13420 |
| NM_031907 | NP_114113 |
| NM_006590 | NP_006581 |
| XM_032614 | XP_032614 |
| BC026072 | AAH26072 |
| XM_033922 | XP_033922 |
| BC000263 | AAH00263 |
| AK022574 | BAB14107 |
| AF035620 | AAC24200 |
| XM_038934 | XP_038934 |
| NM_017414 | NP_059110 |
| XM_165948 | XP_165948 |
| XM_033017 | XP_033017 |
| NM_022832 | NP_073743 |
| XM_113381 | XP_113381 |
| NM_015247 | NP_056062 |
| Y13619 | CAA73941 |
| XM_005624 | XP_005624 |
| XM_165946 | XP_165946 |
| XM_003288 | XP_003288 |
| AK022864 | BAB14279 |
| XM_042698 | XP_042698 |
| AB040886 | BAA95977 |
| XM_115909 | XP_115909 |
| AF077040 | AAD27773 |
| AK022759 | BAB14232 |
| NM_004652 | NP_004643 |
| NM_032147 | NP_115523 |
| NM_006044 | NP_006035 |
| NM_020886 | NP_065937 |
| XM_093148 | XP_093148 |
| AB067478 | BAB67784 |
| XM_036729 | XP_036729 |
| XM_030130 | XP_030130 |
| XM_050754 | XP_050754 |
| NM_032582 | NP_115971 |
| NM_021906 | NP_068706 |
| BC009452 | AAH09452 |
| A8037793 | BAA92610 |
| XM_027038 | XP_027038 |
| XM_034123 | XP_034123 |
| XM_007903 | XP_007903 |
| AK024318 | BAB14881 |
| AK027820 | BAB55392 |
| AB020656 | BAA74872 |
| NM_015017 | NP_055832 |
| XM_166526 | XP_166526 |
| XM_093964 | XP_093964 |
| XM_027791 | XP_027791 |
| NM_006768 | NP_006759 |
| NM_006676 | NP_006667 |
| XM_027039 | XP_027039 |
| XM_165973 | XP_165973 |
| XM_068007 | XP_068007 |
| AK055188 | BAB70869 |
| NM_004651 | NP_004642 |
| XM_051386 | XP_051386 |
| AF017306 | AAC27356 |
| BC018113 | AAH18113 |
| XM_058840 | XP_058840 |
| NM_025090 | NP_079366 |
| XM_028405 | XP_028405 |
| AK027362 | BAB55063 |
| XM_046769 | XP_046769 |
| NM_032236 | NP_115612 |
| NM_032663 | NP_116052 |
| AF000986 | AAC51833 |
| NM_016572 | NP_057656 |
| XM_114325 | XP_114325 |
| NM_032557 | NP_115946 |
| NM_005151 | NP_005142 |
| XM_068006 | XP_068006 |
| NM_006537 | NP_006528 |
| BC022094 | AAH22094 |
| AF233442 | AAF61308 |
| AB033029 | BAA86517 |
| D80012 | BAA11507 |
| AK001671 | BAA91825 |
| AF161450 | AAF29010 |
| XM_093962 | XP_093962 |
| NM_012475 | NP_036607 |
| XM_047413 | XP_047413 |
| AF153604 | AAD41086 |
| NM_006447 | NP_006438 |
| NM_005154 | NP_005145 |
| BC000350 | AAH00350 |
| AF174499 | AAF36540 |
| BC011576 | AAH11576 |
| AF155116 | AAD42882 |
| AF113219 | AAG39290 |
| AK026930 | BAB15591 |
| XM_033651 | XP_033651 |
| BC016663 | AAH16663 |
| XM_167944 | XP_167944 |
| BC015930 | AAH15930 |
| AF079564 | AAC28392 |
| NM_003481 | NP_003472 |
| NM_013396 | NP_037528 |
| AB040948 | BAA96039 |
| AB011142 | BAA25496 |
| XM_049683 | XP_049683 |
| BC025317 | AAH25317 |
| AF161542 | AAF29029 |
| AJ012755 | CAA10171 |
| NM_003940 | NP_003931 |
| XM_034147 | XP_034147 |
| AK057992 | BAB71627 |
| AY008763 | AAG33252 |
| AF335474 | AAK69630 |
| NM_015670 | NP_056485 |
| AB051494 | BAB21798 |
| XM_114357 | XP_114357 |
| BC028583 | AAH28583 |
| AB018340 | BAA34517 |
| XM_011455 | XP_011455 |
| NM_014554 | NP_055369 |
| BC008589 | AAH08589 |
| BC030705 | AAH30705 |
| AF308450 | AAL06294 |
| XM_084114 | XP_084114 |
| XM_058689 | XP_058689 |
| XM_113930 | XP_113930 |
| AB037752 | BAA92569 |
| AK027599 | BAB55222 |
| NM_021627 | NP_067640 |
| NM_020654 | NP_065705 |
| AB051514 | BAB21818 |
| AF199458 | AAL25651 |
| AF217504 | AAG09703 |
| NM_015571 | NP_056386 |
| XM_034262 | XP_034262 |

EMSEMBL Accession No.s

| | |
|---|---|
| ENST00000264281 | ENSP00000264281 |
| ENST00000281393 | ENSP00000281393 |
| ENST00000279003 | ENSP00000279003 |
| ENST00000296943 | ENSP00000296943 |
| ENST00000253105 | ENSP00000253105 |
| ENST00000241470 | ENSP00000241470 |
| ENST00000262306 | ENSP00000262306 |
| ENST00000285285 | ENSP00000285285 |
| ENST00000299678 | ENSP00000299678 |
| ENST00000250495 | ENSP00000250495 |
| ENST00000300630 | ENSP00000300630 |
| ENST00000294574 | ENSP00000294574 |
| ENST00000275108 | ENSP00000275108 |
| ENST00000259937 | ENSP00000259937 |
| ENST00000218299 | ENSP00000218299 |
| ENST00000286669 | ENSP00000286669 |
| ENST00000247526 | ENSP00000247526 |
| ENST00000291615 | ENSP00000291615 |
| ENST00000294270 | ENSP00000294270 |
| ENST00000274459 | ENSP00000274459 |
| ENST00000218154 | ENSP00000218154 |
| ENST00000258728 | ENSP00000258728 |
| ENST00000229699 | ENSP00000229699 |
| ENST00000003302 | ENSP00000003302 |
| ENST00000209500 | ENSP00000209500 |
| ENST00000215794 | ENSP00000215794 |
| ENST00000218348 | ENSP00000218348 |
| ENST00000219473 | ENSP00000219473 |
| ENST00000219689 | ENSP00000219689 |
| ENST00000226440 | ENSP00000226440 |
| ENST00000229268 | ENSP00000229268 |
| ENST00000232487 | ENSP00000232487 |
| ENST00000250066 | ENSP00000250066 |
| ENST00000251722 | ENSP00000251722 |
| ENST00000251784 | ENSP00000251784 |
| ENST00000252403 | ENSP00000252403 |
| ENST00000254181 | ENSP00000254181 |
| ENST00000257011 | ENSP00000257011 |
| ENST00000257548 | ENSP00000257548 |
| ENST00000258123 | ENSP00000258123 |
| ENST00000258399 | ENSP00000258399 |
| ENST00000258499 | ENSP00000258499 |
| ENST00000259103 | ENSP00000259103 |
| ENST00000259404 | ENSP00000259404 |
| ENST00000260187 | ENSP00000260187 |
| ENST00000260188 | ENSP00000260188 |
| ENST00000260419 | ENSP00000260419 |
| ENST00000261497 | ENSP00000261497 |
| ENST00000261601 | ENSP00000261601 |
| ENST00000261737 | ENSP00000261737 |
| ENST00000261843 | ENSP00000261843 |
| ENST00000262773 | ENSP00000262773 |
| ENST00000263184 | ENSP00000263184 |
| ENST00000263311 | ENSP00000263311 |
| ENST00000263858 | ENSP00000263858 |
| ENST00000263966 | ENSP00000263966 |
| ENST00000264208 | ENSP00000264208 |
| ENST00000265452 | ENSP00000265452 |
| ENST00000265560 | ENSP00000265560 |
| ENST00000265831 | ENSP00000265831 |
| ENST00000268049 | ENSP00000268049 |
| ENST00000269134 | ENSP00000269134 |
| ENST00000271487 | ENSP00000271487 |
| ENST00000276019 | ENSP00000276019 |
| ENST00000276060 | ENSP00000276060 |
| ENST00000280377 | ENSP00000280377 |
| ENST00000280395 | ENSP00000280395 |
| ENST00000282088 | ENSP00000282088 |
| ENST00000282344 | ENSP00000282344 |
| ENST00000284174 | ENSP00000284174 |
| ENST00000285199 | ENSP00000285199 |
| ENST00000285679 | ENSP00000285679 |
| ENST00000285681 | ENSP00000285681 |
| ENST00000286782 | ENSP00000286782 |
| ENST00000289865 | ENSP00000289865 |
| ENST00000292729 | ENSP00000292729 |
| ENST00000294383 | ENSP00000294383 |
| ENST00000294617 | ENSP00000294617 |
| ENST00000295040 | ENSP00000295040 |
| ENST00000295041 | ENSP00000295041 |
| ENST00000296572 | ENSP00000296572 |
| ENST00000297228 | ENSP00000297228 |
| ENST00000297229 | ENSP00000297229 |
| ENST00000298462 | ENSP00000298462 |
| ENST00000299574 | ENSP00000299574 |
| ENST00000300924 | ENSP00000300924 |

In a preferred embodiment, variants of deubiquitinating agents have an overall amino acid sequence identity of preferably greater than about 75%, more preferably greater than about 80%, even more preferably greater than about 85% and most preferably greater than 90% of the amino acid sequence. In some embodiments the sequence identity will be as high as about 93 to 95 or 98%.

As is known in the art, a number of different programs can be used to identify whether a protein (or nucleic acid) has sequence identity or similarity to a known sequence; and such sequence identity and/or similarity can be determined using standard techniques known in the art as described herein and above. Further, sequences encoding a deubiquitinating agent may also be used to make variants thereof that are suitable for use in the methods and compositions of the present invention. The deubiquitinating agents and variants suitable for use in the methods and compositions of the present invention may be made as described herein.

In a preferred embodiment, the deubiquitinating agent or variant thereof comprises an amino acid sequence or a nucleic acid sequence of sequence depicted in Table 1. Some of these sequences were generated using the Genscan application available at the Web site www.ensemble.org and a Markov model, and a Gibbs sampling and genetic algorithm. This approach can be used to identify the sequences of deubiquitinating agents or variants thereof; or to identify the sequences of potential deubiquitinating agents or variants thereof.

In a preferred embodiment, the deubiquitinating agent comprises a HIS box amino acid sequence where the two conserved histidines of the HIS box are separated by 7 or 8 residues and more preferably, the HIS box comprises from amino to carboxyl terminus the following amino acid sequence: Y-x-L-x-[SAG]-[LIVMFT]-x(2)-H-x-G-x(4,5)-G-H-Y; and further comprises a CYS box amino acid sequence containing the conserved catalytic cysteine, and preferably other conserved flanking amino acid residues, or homologous amino acid sequence that that is minimally 50-80 amino acids in length. In a preferred embodiment, the deubiquitinating agent comprises, from amino to carboxyl terminus, the following amino acid sequence: G-[LIVMFY]-x(1,3)-[AGC]-[NASM]-x-C-[FYW]- [LIVMFC]-[NST]-[SACV]-x-[LIVMS]-Q [C], where the underlined C is the active site cysteine.

In a preferred embodiment, the deubiquitinating agent comprises a HIS box amino acid sequence or subdomain of a ubiquitin protease containing a conserved his and asp separated by 14 residues, and more preferably has the following amino acid sequence, from amino to carboxl terminus: -(A,V,I)-(K,R,D)-(E,T,D)-(E,M,K)-(D,E,V)-(A,D,N)-(F,L)-H-F-(V,I)-(S,A,L)-(Y,L,F)-(V,N)-(P,H,N)-(V,I)-(N,T,D)(G-(R,H)-L-(Y,F)-E-L-D-(G or other amino acid or no amino acid)-(L,R)-(E,V,P)-(G,Y,F)-P-(I,V)-(D,N)-(H,L)-(G-(A,P,E)-(C,W,T,S)-(N,G,S)-(Q,E,D)-(D,E)-; and further comprises a CYS box that comprises the following amino acid sequence, from amino to carboxyl terminus: (S or other amino acid or no amino acid)-(V,S, or other amino acid or no amino acid)-(V,I,Q, or other amino acid or no amino acid)-(D,Q,G, or other amino acid or no amino acid)-(D,Q, or other amino acid or no amino acid)-(D,S, or other amino acid or no amino acid)-(l,r,v, or other amino acid or no amino acid)-(V,L,T or other amino acid or no amino acid)-(N,D,S or other amino acid or no amino acid)-(N,T,S, or other amino acid or no amino acid)-(M,I,V, or other amino acid or no amino acid)-(F,Y, or other amino acid or no amino acid)-(F, or other amino acid or no amino acid)-(A,M)-(H,K)-Q-L,V,T)-I-(P,N,S,G)-N-(A,S)-C-(A,G)-T-(H,Q,I)-(A,G)-(I,L)-(L,I,V)-(S,H)-(V,A)-(L,I,V)-(S,H)-(V,A)-(L,I,V)-(L,A)-N-(C,N)-.

In another preferred embodiment, the deubiquitinating agent is a human ubiquitin protease, for example, UCH-L3, UCH-L1, similar to C. elegans 37.7 kD protein, BRCA1 associated protein 1, or CGI-70. Table 1 provides accession numbers corresponding to amino acid sequences and their encoding nucleic acid sequences for preferred human deubiquitinating agents.

Deubiquitinating agents, ubiquitin moieties, ubiquitin agents, and target molecules suitable for use in the methods and compositions of the present invention can be cloned and expressed as described herein and below. Thus, probe or degenerate polymerase chain reaction (PCR) primer sequences may be used to find other related or variant deubiquitinating agents, ubiquitin moieties, ubiquitin agents, and target proteins from humans or other organisms. As will be appreciated by those in the art, particularly useful probe and/or PCR primer sequences include the unique areas of a nucleic acid sequence. As is generally known in the art, preferred PCR primers are from about 15 to about 35 nucleotides in length, with from about 20 to about 30 being preferred, and may contain inosine as needed. The conditions for the PCR reaction are well known in the art. In a preferred embodiment, RT-PCR is employed. It is therefore also understood that provided along with the sequences cited herein are portions of those sequences, wherein unique portions of 15 nucleotides or more are particularly preferred. The skilled artisan can routinely synthesize or cut a nucleotide sequence to the desired length.

Once isolated from its natural source, e.g., contained within a plasmid or other vector or excised therefrom as a linear nucleic acid segment, the recombinant nucleic acid can be further-used as a probe to identify and isolate other nucleic acids. It can also be used as a “precursor” nucleic acid to make modified or variant nucleic acids and proteins.

Using the nucleic acids of the present invention which encode a protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence, similarly for proteins. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. As another example, operably linked refers to DNA sequences linked so as to be contiguous, and, in the case of a secretory leader, contiguous and in reading frame. However, enhancers do not have to be contiguous. Linking is accomplished by ligation at convenient restriction sites. If such sites do not exist, the synthetic oligonucleotide adapters or linkers are used in accordance with conventional practice. The transcriptional and translational regulatory nucleic acid will generally be appropriate to the host cell used to express the protein; for example, transcriptional and translational regulatory nucleic acid sequences from Bacillus are preferably used to express the protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

A preferred expression vector system is a retroviral vector system such as is generally described in PCT/US97/01019 and PCT/US97/01048, both of which are hereby expressly incorporated by reference.

Proteins of the present invention are produced by culturing a host cell transformed with an expression vector containing nucleic acid encoding the protein, under the appropriate conditions to induce or cause expression of the protein. The conditions appropriate for protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archaebacteria, fungi, and insect and animal cells, including mammalian cells. Of particular interest are Drosophila melanogaster cells, Pichia pastoris and P. methanolica, Saccharomyces cerevisiae and other yeasts, E. coli, Bacillus subtilis, SF9 cells, SF21 cells, C129 cells, Saos-2 cells, Hi-5 cells, 293 cells, Neurospora, BHK, CHO, COS, and HeLa cells. Of greatest interest are Pichia pastoris and P. methanolica, E. coli, SF9 cells, SF21 cells and Hi-5 cells.

In a preferred embodiment, the proteins are expressed in mammalian cells. Mammalian expression systems are also known in the art, and include retroviral systems. A mammalian promoter is any DNA sequence capable of binding mammalian RNA polymerase and initiating the downstream (3′) transcription of a coding sequence for a protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase 11 to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, proteins are expressed in bacterial systems. Bacterial expression systems are well known in the art.

A suitable bacterial promoter is any nucleic acid sequence capable of binding bacterial RNA polymerase and initiating the downstream (3′) transcription of the coding sequence of a protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3- 11 nucleotides upstream of the initiation codon.

The expression vector may also include a signal peptide sequence that provides for secretion of the protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatment, electroporation, and others.

In one embodiment, proteins are produced in insect cells. Expression vectors for the transformation of insect cells, and in particular, baculovirus-based expression vectors, are well known in the art.

In a preferred embodiment, proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii P. methanolica and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

The protein may also be made as a fusion protein, using techniques well known in the art. Thus, for example, the protein may be made as a fusion protein to increase expression, or for other reasons. For example, when the protein is a peptide, the nucleic acid encoding the peptide may be linked to other nucleic acid for expression purposes. Similarly, proteins of the invention can be linked to protein labels, such as green fluorescent protein (GFP), red fluorescent protein (RFP), blue fluorescent protein (BFP), yellow fluorescent protein (YFP), etc.

In a preferred embodiment, the protein is purified or isolated after expression. Proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the ubiquitin moiety protein may be purified using a standard anti-ubiquitin moiety antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the protein. In some instances no purification will be necessary.

Once made, the compositions find use in a number of applications, including, but not limited to, assaying for agents that modulate the activity of a deubiquitinating agent. The term “modulate” as used herein with reference to the activity of a deubiquitinating agent refers to the increase or decrease in an activity of a deubiquitinating agent. The skilled artisan will appreciate that agents that modulate the activity of deubiquitinating agents (or “modulators”) may affect, for example, a cellular function, enzymatic activity, or binding activity of the deubiquitinating agent, or affect the interaction between a ubiquitin moiety and a ubiquitin substrate.

By “candidate”, “candidate agent”, “candidate modulator”, “candidate modulating agent” or grammatical equivalents herein is meant any candidate molecule, e.g. a protein (which herein includes a protein, polypeptide, and peptide), small organic or inorganic molecule, polysaccharide, or polynucleotide which are to be tested for the ability to modulate the activity of a deubiquitinating agent. Candidate agents encompass numerous chemical classes. In a preferred embodiment, the candidate agents are small molecules. In another preferred embodiment, the candidate agents are organic molecules, particularly small organic molecules, comprising functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more chemical functional groups.

Candidate agents are obtained from a wide variety of sources, as will be appreciated by those in the art, including libraries of synthetic or natural compounds. As will be appreciated by those in the art, the present invention provides a rapid and easy method for screening any library of candidate modulators, including the wide variety of known combinatorial chemistry-type libraries.

In a preferred embodiment, candidate agents are synthetic compounds. Any number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods. As described in WO 94/24314, one of the advantages of the present method is that it is not necessary to characterize the candidate agent prior to the assay. Using the methods of the present invention, any candidate agents can be screened for the ability to increase or decease the activity of a ubiquitin agent, or more specifically for the ability to increase or decrease the attachment of ubiquitin moiety to a substrate. In addition, as is known in the art, coding tags using split synthesis reactions may be used to essentially identify the chemical moieties tested.

Alternatively, a preferred embodiment utilizes libraries of natural compounds, as candidate agents, in the form of bacterial, fungal, plant and animal extracts that are available or readily produced.

Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In a preferred embodiment, candidate agents include proteins, nucleic acids, and chemical moieties, mosr preferably organic chemicals.

In a preferred embodiment, the candidate agents are proteins, as defined above. In a preferred embodiment, the candidate agents are naturally occurring proteins or fragments of naturally occurring proteins. Thus, for example, cellular extracts containing proteins, or random or directed digests of proteinaceous cellular extracts, may be tested, as is more fully described below. In this way libraries of prokaryotic and eukaryotic proteins may be made for screening against any number of candidate agents. Particularly preferred in this embodiment are libraries of bacterial, fungal, viral, and mammalian proteins, with the latter being preferred, and human proteins being especially preferred.

In a preferred embodiment, the candidate agents are peptides of from about 2 to about 50 amino acids, with from about 5 to about 30 amino acids being preferred, and from about 8 to about 20 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

The library should provide a sufficiently structurally diverse population of randomized agents to effect a probabilistically sufficient range of diversity to allow interaction with a particular ubiquitin ligating agent enzyme. Accordingly, an interaction library must be large enough so that at least one of its members will have a structure that interacts with a ubiquitin agents or other components of a ubiquitin reaction, for example, ubiquitin moiety or target protein. Although it is difficult to gauge the required absolute size of an interaction library, nature provides a hint with the immune response: a diversity of 10⁷-10⁸ different antibodies provides at least one combination with sufficient affinity to interact with most potential antigens faced by an organism. Published in vitro selection techniques have also shown that a library size of 10⁷to 10⁸ is sufficient to find structures with affinity for a target. A library of all combinations of a peptide 7 to 20 amino acids in length, such as generally proposed herein, has the potential to code for 20⁷ (10⁹) to 20²⁰. Thus, with libraries of 10⁷ to 10⁸ different molecules the present methods allow a “working” subset of a theoretically complete interaction library for 7 amino acids, and a subset of shapes for the 20²⁰ library. Thus, in a preferred embodiment, at least 10⁶, preferably at least 10⁷, more preferably at least 10⁸ and most preferably at least 10⁹ different sequences are simultaneously analyzed in the subject methods. Preferred methods maximize library size and diversity.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

In a preferred embodiment, the bias is towards peptides or nucleic acids that interact with known classes of molecules. For example, when the candidate agent is a peptide, it is known that much of intracellular signaling is carried out via short regions of polypeptides interacting with other polypeptides through small peptide domains. For instance, a short region from the HIV-1 envelope cytoplasmic domain has been previously shown to block the action of cellular calmodulin. Regions of the Fas cytoplasmic domain, which shows homology to the mastoparan toxin from Wasps, can be limited to a short peptide region with death-inducing apoptotic or G protein inducing functions. Magainin, a natural peptide derived from Xenopus, can have potent anti-tumor and anti-microbial activity. Short peptide fragments of a protein kinase C isozyme (βPKC), have been shown to block nuclear translocation of βPKC in Xenopus oocytes following stimulation. And, short SH-3 target peptides have been used as psuedosubstrates for specific binding to SH-3 proteins. This is of course a short list of available peptides with biological activity, as the literature is dense in this area. Thus, there is much precedent for the potential of small peptides to have activity on intracellular signaling cascades. In addition, agonists and antagonists of any number of molecules may be used as the basis of biased randomization of candidate modulators as well.

Thus, a number of molecules or protein domains are suitable as starting points for the generation of biased randomized candidate modulators. A large number of small molecule domains are known, that confer a common function, structure or affinity. In addition, as is appreciated in the art, areas of weak amino acid homology may have strong structural homology. A number of these molecules, domains, and/or corresponding consensus sequences, are known, including, but are not limited to, SH-2 domains, SH-3 domains, Pleckstrin, death domains, protease cleavage/recognition sites, enzyme inhibitors, enzyme substrates, and Traf.

In a preferred embodiment, the candidate modulating agent is a polypeptide. In another preferred embodiment, the polypeptide is a cyclic peptide having at least 4 and up to 20 or more amino acids. Also in another preferred embodiment, the polypeptide is a catalytically inactive polypeptide. Examples of catalytically inactive polypeptides include, but are not limited to, catalytically inactive deubiquitinating agent and, more specifically a catalytically inactive UBP, UCH, SENP or JAMM-CP.

In another embodiment, the candidate modulating agent is a peptide fragment of a polypeptide of the ubiquitin complex. In another aspect, the candidate modulating agent is a peptide fragment of a fulllength cleavable ubiquitin fusion polypeptide (see, e.g., U.S. Ser. No. 09/800,770, filed Mar. 6, 2001, expressly incorporated herein by reference).

In a preferred embodiment, the candidate agents are nucleic acids. With reference to candidate agents, by “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl etal., Eur. J. Biochem. 81:579 (1977); Letsinger etal., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et aL, J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et aL., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y.S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y.S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2,1997 page 35. All of these references are hereby expressly incorporated by reference. These modifications of the ribose-phosphate backbone may be done to facilitate the addition of additional moieties such as labels, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made. Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribonucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

As described above generally for proteins, nucleic acid candidate agent may be naturally occurring nucleic acids, random nucleic acids, or “biased” random nucleic acids. For example, digests of prokaryotic or eukaryotic genomes may be used as is outlined above for proteins. Where the ultimate expression product is a nucleic acid, at least 10, preferably at least 12, more preferably at least 15, most preferably at least 21 nucleotide positions need to be randomized, with more preferable if the randomization is less than perfect. Similarly, at least 5, preferably at least 6, more preferably at least 7 amino acid positions need to be randomized; again, more are preferable if the randomization is less than perfect.

In a preferred embodiment, the candidate modulating agent is a mutant cDNA encoding a catalytically inactive polypeptide. Examples of such catalytically inactive polypeptides include, but are not limited to, catalytically inactive deubiquitinating agents and, more specifically, catalytically inactive UBP, UCH, SENP or JAMM-CP.

In a preferred embodiment, the candidate modulating agent is an RNA, for example an antisense RNA or siRNA (small inhibitory RNA). In another preferred embodiment, the siRNA inhibits translation of mRNA encoding a deubiquitinating agent, for example a UBP, UCH, SENP or JAMMCP. The siRNAs can be prepared using the methods described herein and known in the art.

In a preferred embodiment, the candidate agents are organic moieties. In this embodiment, as is generally described in WO 94/24314, candidate agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

As will be appreciated by those in the art, it is possible to screen more than one type of candidate agent at a time. Thus, the library of candidate agents used may include only one type of agent (i.e. peptides), or multiple types (peptides and organic agents). The assay of several candidates at one time is further discussed below.

The present invention provides methods and compositions comprising combining different combinations of ubiquitin agents, with ubiquitin moiety, in the presence or absence of a target protein. In preferred embodiments, a candidate agent is included in the combining to assay for an agent that modulates the attachment of a ubiquitin moiety to a substrate molecule. In preferred embodiments the ubiquitin moiety and/or the substrate molecule of interest in the assay comprises a tag.

Preferably the tag is a label, a partner of a binding pair, or a substrate binding molecule (or attachment tag). In a preferred embodiment, the tag is an epitope tag. In another preferred embodiment, the tag is a label. More preferably, the tag is a fluorescent label or a binding pair partner. In a preferred embodiment, the tag is a binding pair partner and the ubiquitin moiety is labeled by indirect labeling. In the indirect labeling embodiment, preferably the label is a fluorescent label or a label enzyme. In an embodiment comprising a label enzyme, preferably the substrate for that enzyme produces a fluorescent product. In a preferred embodiment, the label enzyme substrate is luminol. In a preferred embodiment, combining specifically excludes combining the components with a target protein.

In another preferred embodiments, a preferred combination is Tag1-ubiquitin moiety, tag2-ubiquitin moiety. Preferably, tag1 and tag2 are labels, preferably fluorescent labels, most preferably tag1 and tag2 constitute a FRET pair. In a preferred embodiment, a preferred combination is tag1-ubiquitin moiety and tag2-substrate molecule of interest. Preferably, tag1 is a label, a partner of a binding pair, or a substrate binding molecule and tag2is a different label, partner of a binding pair, or substrate binding molecule. More preferably, tag1 is a fluorescent label or a member of a binding pair. When tag1 is a member of a binding pair, preferably tag1 is indirectly labeled. Still more preferably, tag-1 is indirectly labeled with a label enzyme. Preferably the label enzyme substrate used to reveal the presence of the enzyme produces a fluorescent product, and more preferably is luminol. In the presently described combination, preferably tag2 is a surface substrate binding element, more preferably a His-tag.

In a preferred embodiment, the ubiquitin complex does not comprise a target protein. In a preferred embodiment, a mono- or poly-ubiquitin moiety is a ubiquitin substrate molecule, as discussed above.

Because the different combinations of ubiquitin agents are specific for particular target proteins, the present assays are more versatile then conventional assays which require a target protein.

The components of the present assays may be combined in varying amounts. In a preferred embodiment, ubiquitin moiety is combined at a final concentration of from 20 to 200 ng per 100 μl reaction solution, most preferable at about 100 ng per 100 μl reaction solution.

In a preferred embodiment, the deubiquitinating agent is combined at a final concentration of from 1 ng to 500 ng per 100 μl reaction solution, more preferably from 50 to 400 ng per 100 μl reaction solution, still more preferably from 100 to 300 ng per 100 μl reaction solution, and still more preferably about 100 ng per 100 μl reaction solution. The skilled artisan will recognize that optimum concentrations of components are easiliy determined by routine experimentation.

In preferred embodiments, the components of the present assays are combined under reaction conditions that favor the activity of the deubiquitinating agents of the present invention. Generally, this will be under physiological conditions. Incubations may be performed at any temperature which facilitates optimal activity, typically between 4 and 40° C. Incubation periods are selected for optimum activity, but may also be optimized to facilitate rapid high through put screening. Typically between 0.5 and 1.5 hours will be sufficient.

A variety of other reagents may be included in the compositions. These include reagents like salts, solvents, buffers, neutral proteins, e.g. albumin or detergents which may be used to facilitate the optimal activity of deubiquitinating agents; and/or reduce non-specific or background interactions. Also reagents that otherwise improve the efficiency of the assay, such as nuclease inhibitors, antimicrobial agents, etc., may be used.

The combining of components in a reaction mixture may be added in any order that promotes deubiquitinating activity by deubiquitinating agents, and more specifically promotes the cleavage of ubiquitin moiety in the assay; or optimizes identification of the modulating activity of a candidate modulating agent. In a preferred embodiment, a ubiquitin complex is provided in a reaction buffer solution, followed by addition of a deubiquitinating agent. In an alternate preferred embodiment, ubiquitin complex is provided in a reaction buffer solution, a candidate modulating agent is then added, followed by the addition of a deubiquitinating agent.

Once combined, in a preferred embodiment, the amount of ubiquitin moiety cleaved or released from the ubiquitin complex is measured. As will be understood by one of ordinary skill in the art, the mode of measuring may depend on the specific tag attached to the ubiquitin moiety. As will also be apparent to the skilled artisan, the amount of ubiquitin moiety attached to a substrate molecule will encompass not only the particular ubiquitin moiety bound directly to the substrate molecule, but also a mono- or poly-ubiquitin moiety preferably attached to the substrate molecule.

In a preferred embodiment, the tag attached to the ubiquitin moiety is a fluorescent label. In a preferred embodiment, the tag attached to ubiquitin moiety is an enzyme label or a binding pair member which is indirectly labeled with an enzyme label. In this latter preferred embodiment, the enzyme label substrate produces a fluorescent reaction product. In these preferred embodiments, the amount of ubiquitin moiety bound is measured by luminescence.

In other preferred embodiments, at least a first and a second ubiquitin moiety is used, wherein the first and second ubiquitin moieties comprise different fluorescent labels, and wherein the labels form a FRET pair.

As used herein, luminescence” or “fluorescent emission” means photon emission from a fluorescent label. In an embodiment where FRET pairs are used, fluorescence measurements may be taken continuously or at time-points during the ligation reaction. The skilled artisan will recognize that a FRET pair comprising a fluorophor and a quencher on either side of a cleavable bond will provide increasing fluorescence as the deubiquitination proceeds, while a similarly situated FRET pair comprising two fluorphors will result in decreasing fluorescence as deubiquitinaion proceeds. Equipment for such measurement is commercially available and easily used by one of ordinary skill in the art to make such a measurement.

Other modes of measuring the cleavage of ubiquitin moiety from a substrate molecule are well known in the art and easily identified by the skilled artisan for each of the labels described herein. For example, radioisotope labeling may be measured by scintillation counting, or by densitometry after exposure to a photographic emulsion, or by using a device such as a Phosphorimager. Likewise, densitometry may be used to measure the cleavage of ubiquitin moiety following a reaction with an enzyme label substrate that produces an opaque product when an enzyme label is used. The skilled artisan will recognize that measurements of activity are generally determined relative to similar conditions but in the absence of deubiquitinating agent or in the presence of an inhibitor. Titrations of the deubiquitinating agent may also be made to provide information on activity.

In a preferred embodiment, the methods of the invention include the use of liquid handling components. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, highdensity transfers, full-plate serial dilutions, and high capacity operation.

In a preferred embodiment, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on,an upgradable modular platform for additional capacity. This modular platform includes a variable speed orbital shaker, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of heat exchangers such as controlled blocks or platforms to provide accurate temperature control of incubating samples from 0° C. to 100° C.

In a preferred embodiment, interchangeable pipet heads (single or multi-channel ) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluroescence resonance energy transfer (FRET), luminescence, quenching, twophoton excitation, and intensity redistribution; CCD cameras to capture and transform data and images into quantifiable formats; and a computer workstation.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic apparatus includes a central processing unit which communicates with a memory and a set of inpuvoutput devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components such as label probes, etc.

In a preferred embodiment, a ubiquitin complex of interest in the assays of the present invention is bound to a surface substrate. This may be achieved as described above for the binding of a label to ubiquitin moiety.

In another preferred embodiment, a ubiquitin conjugating agent is bound to a surface substrate in the absence of a ubiquitin ligating agent. This may be achieved, as described above for the binding of a label to ubiquitin moiety. This may also be accomplished using tag-ubiquitin conjugating agent, wherein the tag is a surface substrate binding molecule.

In another preferred embodiment, a ubiquitin ligating agent is bound to a surface substrate in the absence of a target protein. This may be achieved, as described above for the binding of a label to ubiquitin moiety. This may also be accomplished using tag-ubiquitin ligating agent, wherein the tag is a surface substrate binding molecule.

In general, any substrate binding molecule can be used. In a preferred embodiment, the tag is a His-tag and the surface substrate is nickel. In a preferred embodiment, the nickel surface substrate is present on the surface of the wells of a multi-well plate, such as a 96 well plate. Such multi-well plates are commercially available. The binding of the enzyme to a surface substrate facilitates the separation of bound ubiquitin moiety from unbound ubiquitin moiety. In the present embodiment, the unbound ubiquitin moiety is easily washed from the receptacle following the ligation reaction. As will be appreciated by those of skill in the art, the use of any surface substrate binding element and receptacle having the surface substrate to which it binds will be effective for facilitating the separation of bound and unbound ubiquitin moiety.

In an alternative embodiment, the substrate molecule of interest in the assays of the present invention comprise a bead that is attached to, at most, all but one member of a ubiquitin complex, preferably only one member of a ubiquitin complex, directly or via a substrate binding element. Following cleavage, the beads may be separated from the unbound ubiquitin moiety and the bound ubiquitin moiety measured. In a preferred embodiment, the ubiquitin complex of interest in the assay of the present invention comprises a bead and a second member of the ubiquitin complex comprises a tag, wherein the tag is a fluorescent label. In this embodiment, the beads with bound ubiquitin moiety may be separated using a fluorescence-activated cell sorting (FACS) machine. Methods for such use are described, e.g., in U.S. patent application Ser. No. 09/047,119; and Boisclair et al. (2000) J. Biomol. Screening 5(5):319-328, each hereby incorporated in its entirety. The amount of bound ubiquitin moiety can then be measured.

The beads may also be independently labeled and sorted as well. In a preferred embodiment, different ubiquitin comples, such as complexes comprising different ubiquitin moieties, are attached to differently labeled and sortable substrates, such as beads, such that each particular ubiquitin moiety is attached to a particularly labeled substrate. The ubiquitin moiety/substrates may then be exposed, even as a mixture, to a deubiquitinating agent, whereby a preferred substrate (ubiquitin moiety) for the deubiquitinating agent may be identified.

In another embodiment, the ubiquitin moiety is bound to a surface substrate. Preferably in this embodiment, the assays comprise a tag1-ubiquitin moiety-tag2, wherein tag1 and tag2 are attached to the ubiquitin complex on opposite sides of the cleavable bond, e.g., attached to different members of the ubiquitin complex. Preferably, tag1 and tag2 are labels, preferably fluorescent labels, most preferably tag1 and tag2 constitute a FRET pair. In this embodiment, the deubiquitinating activity is measured by measuring the fluorescent emission spectrum. This measuring may be continuous or at one or more times following the combination of the components. Alteration in the fluorescent emission spectrum of the ubiquitin complex in the presence of a deubiquitinating enzyme as compared with in the absence of or in the additional presence of an inhibitor of the deubiquitinating agent indicates the amount of deubiquitination. The skilled artisan will appreciate that in this embodiment, alteration in the fluorescent emission spectrum results from ubiquitin moiety bearing different members of the FRET pair being brought into close proximity, for example, on different ubiquitins in a poly-ubiquitin moiety and/or on a ubiquitin moiety and a target protein near the ubiquitin moiety binding locations.

In one preferred embodiment of the present methods, the ubiquitin complex comprises an E3, for example an MdM2 protein, which E3 comprises a first FRET label and a ubiquitin moiety which comprises a second FRET label. In another embodiment, the E3 comprises an attachment tag. In another embodiment, the E3 is preferably provided on a solid support, and more preferably the solid support comprises a microtiter plate or a bead. In another embodiment, the E3 protein is preferably a mammalian mdm2 and more preferably a human mdm2.

In another preferred embodiment, the ubiquitin complex comprises a target protein, such as p53, which E3 comprises a first FRET label, and the ubiquitin complex comprises a ubiquitin moiety comprising a second FRET label.

In another embodiment, the target protein preferably comprises an attachment tag. In another embodiment, the target protein is preferably provided on a solid support, and more preferably the solid support comprises a microtiter plate or a bead.

In a preferred embodiment, the compositions of the invention are used to identify candidate modulating agents that modulate the deubiquitinating activity of a deubiquitinating agent. In this embodiment, the composition includes a candidate modulating agent. In a preferred embodiment, the measured amount and/or rate of tag-ubiquitin moiety released or cleaved from ubiquitin substrate or cleavable ubiquitin fusion polypeptide is compared with that when the candidate modulating agent is absent from the ubiquitin substrate or cleavable ubiquitin fusion polypeptide, respectively, whereby the presence or absence of the agent's effects on the deubiquitinating activity is determined. In this embodiment, it is determined whether the candidate modulating agent enhances or inhibits, or reduces or increases the deubiquitinating activity of the deubiquitinating agent.

In a preferred embodiment, cleavage or release of ubiquitin moiety from an E2 of a ubiquitin complex comprising a poly ubiquitin moiety or a ubiquin moiety attached, via an isopeptide bond, to the E2, is measured. This embodiment may also comprise the step of comparing the amount and/or rate of ubiquitin moiety cleaved or released from the ubiquitin complex in a composition comprising a candidate agent, whereby the modulating activity of the candidate agent is determined.

In another preferred embodiment, the compositions of the invention are used to identify candidate modulating agents that modulate the cleavage or release of a ubiquitin moiety from a ubiquitin complex comprising a ubiquitin moiety attached to a ubiquitin substrate. In this embodiment, the present assays include a candidate modulating agent. In a preferred embodiment, where tag1 and tag2 constitute a FRET pair, the measured amount and/or rate of tag1-ubiquitin moiety and tag2-ubiquitin moiety released or cleaved from the ubiquitin substrate (as a poly-ubiquitin moiety and/or ubiquitin moiety cleaved or released from the ubiquitin substrate molecule) is compared with the amount or rate of such cleavage or release in the absence of the candidate modulating agent, whereby the presence or absence of the candidate modulating agent's effect on the cleavage or release of ubiquitin moiety from the ubiquitin complex is determined. In this embodiment, it is determined whether the candidate agent enhances or inhibits, or increases or decreases, the cleavage or release of the ubiquitin moiety from the ubiquitin complex.

In a preferred embodiment, multiple assays are performed simultaneously in a high throughput screening system. In this embodiment, multiple assays may be performed in multiple receptacles, such as the wells of a 96 well plate or other multi-well plate. As will be appreciated by one of skill in the art, such a system may be applied to the assay of multiple candidate modulating agents, multiple ubiquitin complexes, and/or multiple deubiquitinating agents. In an alternate preferred embodiment, the present invention is used in a high throughput screening system for simultaneously testing the effect of individual candidate modulating agents by additionally combining a candidate modulating agent.

In another aspect, the invention provides a method of assaying for the cleavage or release of a ubiquitin moiety from a ubiquitin complex in a reaction mixture or in a cell. In this embodiment, the ubiquitin complex comprises tag1-ubiquitin moiety and tag2-ubiquitin moiety, wherein tag1 and tag2 constitute a FRET pair or tag1 is a fluorescent label and tag2 is a quencher of tag1. Fluorescent emission spectrum is measured as an indication of whether deubiquitinating activity is present in the mixture or cell. In a preferred embodiment, the ubiquitin moiety also comprises a member of a binding pair, such as FLAG. In this latter embodiment, components involved in the deubiquitination can be isolated from the mixture using any one of a number of affinity-based separation means such as fluorescent beads coated with anti-FLAG antibody or amino precipitation using anti-FLAG antibodies, or using anti-FLAG antibody attached to a solid support. Other means of separating or detecting cleaved or released ubiquitin moiety or ubiquitin attached or fused components from components of the cell or mixture will be readily apparent to the skilled artisan. The skilled artisan will appreciate that separation of these components for individual identification or subsequent investigation may be obtained by several means well known in the art, such as by HPLC or electrophoresis.

In a preferred embodiment, the target protein comprises a first FRET label and the ubiquitin moiety comprises a second FRET label. In another preferred embodiment, the target protein comprises a FRET label and the ubiquitin moiety comprises a Quencher. In another preferred embodiment, the target protein comprises an attachment moiety, and in yet another embodiment, the target protein is provided on a solid support, for example a microtiter plate or a bead.

In a preferred embodiment, the ubiquitin complex comprises a ubiquitin ligating agent comprising a first FRET label and a ubiquitin moiety comprising a second FRET label. In another preferred embodiment, the ubiquitin ligating agent comprises a FRET label and the ubiquitin moiety comprises a Quencher, or vice versa. Also in another preferred embodiment, the ubiquitin ligating agent comprises an attachment moiety. In another preferred embodiment, the ubiquitin ligating agent is provided on a solid support, for example, a microtiter plate or a bead.

In another preferred embodiment, the ubiquitin comple comprises a ubiquitin conjugating enzyme comprising a first FRET label and a ubiquitin moiety comprising a second FRET label. Also in another preferred embodiment, the ubiquitin conjugating enzyme comprises a FRET label and the ubiquitin moiety comprises a Quencher, or vice versa. In another preferred embodiment, the ubiquitin conjugating agent comprises an attachment moiety; and in yet another preferred embodiment, the ubiquitin conjugating agent is provided on a solid support, for example a microtiter plate or a bead.

In a preferred embodiment, the ubiquitin complex comprises a cleavable ubiquitin fusion polypeptide comprising at least one tag. In another preferred embodiment, the cleavable ubiquitin fusion polypeptide comprises a first tag and a second tag. Also, in another preferred embodiment, the first tag is at the amino terminus of the cleavable ubiquitin fusion polypeptide and the second tag is at the carboxyl terminus of the cleavable ubiquitin moiety. In another preferred embodiment, the first tag is a first label and the second tag is a second label. Also, in another preferred embodiment wherein there is a first label and a second label, one label is a first FRET label and the other label is a second FRET label; or one label is a FRET label and the other label is a Quencher of the FRET label. In another preferred embodiment, one tag comprises a Flag tag and the other tag comprises a His tag.

In a preferred embodiment, the cleavable ubiquitin fusion polypeptide comprises, from amino to carboxyl terminus, a first ubiquitin moiety comprising a first tag operably linked to a second ubiquitin moiety comprising a second tag. In another preferred embodiment, the first tag is at the amino terminus of the first ubiquitin moiety and the second tag is at the carboxyl terminus of the second ubiquitin moiety and in a further aspect, either the first tag or the second tag is a His tag; or the first tag or the second tag is a GST tag. In another preferred embodiment, the cleavable ubiquitin fusion polypeptide comprises a ubiquitin moiety operably linked to a reporter protein.

In a preferred embodiment, the cleavable ubiquitin fusion polypeptide comprises a ubiquitin moiety operably linked to a reporter protein. In another preferred embodiment, the reporter protein is betagalactosidase or a fluorescent reporter protein, for example, Green Fluorescent Protein (GFP) and preferably, Green Fluorescent Protein (GFP) of a renilla species.

In a preferred embodiment, the assaying is by FACS. In another aspect, the assaying is by high pressure liquid chromatography (HPLC), for example, reverse phase HPLC, and in a further aspect, the assaying is by mass spectromety.

In a preferred embodiment, the present invention provides a method for performing functional deubiquitination screens. In a preferred embodiment, the method comprises contacting a cell with a negative effector of a deubiquitinating agent and screening for an altered phenotype in the cell. By “negative effector” is meant a molecule known or believed to decrease the functional activity of a deubiquitinating agent in a cell. The decrease in functional activity may arise via any mechanism, including through reduction of expression of the deubiquitinating agent, either at the.transcriptional or translational level (e.g., using siRNA or antisense nucleic acid directed against nucleic acid encoding the deubiquitinating agent), competition with an endogenous deubiquitinating agent (e.g., using a variant agent (including substitutions, insertions and deletions) and/or dominant negative mutant of the deubiquitinating agent) or binding and, preferably, interfering with function of a deubiquitinating agent (e.g., using a peptide, cyclic or linear, or other binding molecule such as a small organic molecule).

In an alternate embodiment, the methods include providing a cell culture, whose cells contain a library of nucleic acids comprising nucleic acids encoding at least one negative effector of deubiquitinating agents. The invention further provides screening the cell culture for altered phenotype as compared to control cells, isolating those with altered phenotypes and identifying the negative effector of the deubiquitin agent(s) that resulted in the altered phenotype.

In one embodiment, the invention provides culturing cells expressing or over-expressing different deubiquitinating agents and assaying a functional readout for the activity of the deubiquitinating agents. Modulation of the functional readout indicates involvement of the deubiquitinating agent in that pathway.

In a preferred general embodiment, the methods involve expressing a negative effector of a deubiquitinating agent in a cell system and determining the effect of the variant deubiquitinating agent in a functional assay. The functional assay may involve a cellular readout as described below, or may involve determining the amount of ubiquitin on a target protein. That is, the method involves measuring the amount of ubiquitin moiety attached to at least one of the following substrate molecules: a deubiquitinating agent; a target protein; or a mono- or poly-ubiquitin moiety which is preferably attached to a deubiquitinating agent or target protein.

Accordingly, the compositions of the invention find use in a variety of functional screens. The functional screens are used to elucidate the physiological role of the deubiquitinating agent examined in the screen, i.e., to determine whether a particular deubiquitinating agent is a modulator of a particular function. By “modulator” is meant the ability to enhance or inhibit, or increase or decrease a partuclular functional event. Such information provides instruction for the development of therapies for disease states associated with the function screened. In many instances, the negative effectors of the deubiquitinating agents may serve as therapeutics themselves, or as models for the prduction of therapeutic molecules.

Examples of functional screens are varied, and can include any of a variety of screens including cellular assays. In addition, the functional screens can include biochemical assays such as detecting in increase or decrease in a putative ubiquitin substrate or target molecule.

In any event, in one embodiment the functional screens include expressing in a cell or cell population one ot more deubiquitinating agents or negative effectors thereof, and determining an increase or decrease in a potential ubiquitin substrate or target molecule.

The level of proteins can be examined in any of a variety of methods as are known to those of ordinary skill of the art. These methods include immunoblotting, or detecting labeled proteins, for example His-tagged proteins or radio-labeled proteins, and the like. In addition, protein identification can be accomplished by mass spectrometry. This is particularly useful when the identity of the proteins is unknown.

In a preferred embodiment, the functional screens include detecting a change in cell viability. That is, cells can be cultured expressing a negative effector of a deubiquitinating agent, such as a dominant negative, or wild type deubiquitinating agent . The cultures can be compared to control cultures and the level of cell viability examined. Cell viability can be determined by any of a variety of methods that are known to those of ordinary skill in the art.

In addition, cell cycle progression can be monitored as a function of expression of various wild type or dominant negative mutant deubiquitinating agent. The cell cycle progression can be examined by methods known in the art as described in U.S. patent application Ser. No. 09/157,748, filed Sep. 21, 1998, which is expressly incorporated herein by reference.

Additional functional assays include screening for modulators of IgE as described in more detail in U.S. Ser. Nos. 09/076,624, filed May 12, 1998, 09/963,247, filed Sep. 25, 2001, 60/165,189, filed Nov. 12,1999, 09/963,206, filed Sep. 25, 2001, and 09/966,976, filed Sep. 27, 2001, which are expressly incorporated herein by reference.

Additional functional assays include screening for exocytosis modulators as set forth in 09/062,330, filed Apr. 17,1998, which is expressly incorporated herein by reference.

Additional functional assays include screening for modulators of T-cells and B-cells as set forth and 09/429,578, filed Oct. 28,1999, which is expressly incorporated herein by reference.

Additional functional assays include screening for modulators of angiogenesis, macrophage activation, astrocyte differentiation. Preferred functional assays include but are not limited to cell cycle assays, cell proliferation assays, assays for apoptosis, assays for T-cell and B-cell activation, assays for macrophage and monocyte activation, assays for cell adhesion, assays for ostecloast differentiation, assays for cholesterol metabolism and assays for neurodegenerative disease. These assays are described as cited above and in more detail in the examples. All references are expressly incorporated herein by reference.

The functional assays of the present invention may be useful to screen a large number of cell types under a wide variety of conditions. In one embodiment, host cells are cells that are involved in disease states.

In a preferred embodiment, the present methods are useful in cancer applications. The ability to rapidly and specifically kill tumor cells is a cornerstone of cancer chemotherapy. In general, using the methods of the present invention, a deubiquitinating agent or a negative effector of a deubiquitinating agent can be introduced into any tumor cell (primary or cultured), and deubiquitinating agents can thereby be identified which modulate apoptosis, cell death, loss of cell division or decreased cell growth. In an alternive embodiment, libraries encoding deubiquitinating agents or putative negative effectors of a deubiquitinating agent(s) can be introduced into any tumor cell (primary or cultured), and deubiquitinating agents or negative effector(s) of deubiquitinating agents can be identified which induce apoptosis, cell death, loss of cell division or decreased cell growth.

Alternatively, the methods of the present invention can be combined with other cancer therapeutics (e.g. drugs, such as taxol, or radiation) to sensitize the cells and thus induce rapid and specific apoptosis, cell death, loss of cell division or decreased cell growth after exposure to a secondary agent. Similarly, the present methods may be used in conjunction with known cancer therapeutics to screen for agonists to make the therapeutic more effective or less toxic. This is particularly preferred when the chemotherapeutic is very expensive to produce such as taxol. Other cancer applications are described in more detail in U.S. Ser. No. 09/800,770, filed Mar. 6, 2001, which is expressly incorporated herein by reference.

In a preferred embodiment, the present methods are useful in cardiovascular applications. In a preferred embodiment, cardiomyocytes may be screened for the prevention of cell damage or death in the presence of normally injurious conditions, including, but not limited to, the presence of toxic drugs (particularly chemotherapeutic drugs), for example, to prevent heart failure following treatment with adriamycin; anoxia, for example in the setting of coronary artery occlusion; and autoimmune cellular damage by attack from activated lymphoid cells (for example as seen in post viral myocarditis and lupus). Deubiquitinating agents or negative effectors of deubiquitinating agents can inserted into cardiomyocytes, which cells are subjected to the insult, and deubiquitinating agents are identified which modulate any or all of: apoptosis; membrane depolarization (i.e. decrease arrythmogenic potential of insult); cell swelling; or leakage of specific intracellular ions, second messengers and activating molecules (for example, arachidonic acid and/or lysophosphatidic acid).

In a preferred embodiment, the present methods are used to screen for diminished arrhythmia potential in cardiomyocytes. The screens comprise the introduction of one or more deubiquitinating agents or one or more negative effectors of deubiquitinating agents into the cardiomycytes, followed by the application of arrythmogenic insults, thereby identifying deubiquitinating agents that modulate specific depolarization of cell membrane. This may be detected using patch clamps, or via fluorescence techniques). Similarly, channel activity (for example, potassium and chloride channels) in cardiomyocytes could be regulated using the present methods in order to enhance contractility and prevent or diminish arrhythmias.

In a preferred embodiment, the present methods are used to screen for enhanced contractile properties of cardiomyocytes and diminish heart failure potential. The introduction of one or more deubiquitinating agents, one or more negative effectors of deubiquitinating agents, or libraries thereof, followed by measuring the rate of change of myosin polymerization/depolymerization using fluorescent techniques can be done. Deubiquitinating agents may be identified that modulate this cellular electrochemical flux. An increase in the rate of change of this phenomenon can result in a greater contractile response of the entire myocardium, similar to the effect seen with digitalis.

In a preferred embodiment, the present methods are useful to identify agents that will regulate the intracellular and sarcolemmal calcium cycling in cardiomyocytes in order to prevent arrhythmias. Deubiquitinating agents or negative effectors of deubiquitinating agents are selected that regulate sodium-calcium exchange, sodium proton pump function, and regulation of calcium-ATPase activity.

In a preferred embodiment, the present methods are useful to identify deubiquitinating agents that modulate embolic phenomena in arteries and arterioles leading to strokes (and other occlusive events leading to kidney failure and limb ischemia) and angina precipitating a myocardial infarct. For example, deubiquitinating agents or negative effectors of deubiquitinating agents are identified that will diminish the adhesion of platelets and leukocytes, and thus diminish the occlusion events.

Adhesion in this setting can be inhibited by the deubiquitinating agents, negative effectors, or libraries thereof of the invention being introduced into endothelial cells (quiescent cells, or activated by cytokines, i.e. IL-1, and growth factors, i.e. PDGF/EGF) by screening for deubiquitinating agents or negative effectors of deubiquitinating agents that induce either: 1) down regulation of adhesion molecule expression on the surface of the endothelial cells (binding assay); 2) blockade of adhesion molecule activation on the surface of these cells (signaling assay); or 3) release in an autocrine manner peptides that block receptor binding to the cognate receptor on the adhering cell.

Embolic phenomena can also be addressed by activating proteolytic enzymes on the cell surfaces of endothelial cells, and thus releasing active enzyme which can digest blood clots. Thus, delivery of the deubiquitinating agents, negative effectors of deubiquitinating agents, or libraries thereof, of the invention to endothelial cells is done, followed by standard fluorogenic assays, which will allow monitoring of proteolytic activity on the cell surface towards a known substrate. Deubiquitinating agents can then be identified which modulate activation of specific enzymes towards specific substrates.

In a preferred embodiment, arterial inflammation in the setting of vasculitis and post-infarction can be regulated by decreasing the chemotactic responses of leukocytes and mononuclear leukocytes. This can be accomplished by blocking chemotactic receptors and their responding pathways on these cells. Deubiquitinating agents, negative effectors of deubiquitinating agents, or libraries thereof, can be inserted into these cells, and the chemotactic response to diverse chemokines (for example, to the IL-8 family of chemokines, RANTES) determined in cell migration assays.

In a preferred embodiment, arterial restenosis following coronary angioplasty can be controlled by regulating the proliferation of vascular intimal cells and capillary and/or arterial endothelial cells.

Deubiquitinating agents, negative effectors of deubiquitinating agents, or libraries thereof, can be inserted into these cell types and their proliferation in response to specific stimuli monitored.

The control of capillary and blood vessel growth is an important goal in order to promote increased blood flow to ischemic areas (growth), or to cut-off the blood supply (angiogenesis inhibition) of tumors. Deubiquitinating agents, negative effectors of deubiquitinating agents, or libraries thereof, can be inserted into capillary endothelial cells and their growth monitored. Stimuli such as low oxygen tension and varying degrees of angiogenic factors can regulate the responses, and peptides isolated that produce the appropriate phenotype. Screening for modulation of vascular endothelial cell growth factor, important in angiogenesis, would also be useful.

In a preferred embodiment, the present methods are useful in screening for modulators of atherosclerosis producing mechanisms to find deubiquitinating agents that regulate LDL and HDL metabolism. Deubiquitinating agents, negative effectors of deubiquitinating agents, or libraries thereof, can be inserted into the appropriate cells (including hepatocytes, mononuclear leukocytes, endothelial cells) and deubiquitinating agents can be identified which modulate release of LDL or synthesis of LDL, or conversely release of HDL or synthesis of HDL. Deubiquitinating agents, negative effectors of deubiquitinating agents, or libraries thereof, can also be used to identify deubiquitinating wagents that modulate the production of oxidized LDL, which has been implicated in atherosclerosis and isolated from atherosclerotic lesions. Modulation could occur by altering its expression, modulating reducing systems or enzymes, or affecting the activity or production of enzymes implicated in production of oxidized LDL, such as 15-lipoxygenase in macrophages.

In a preferred embodiment, the present methods are used in screens to identify deubiquitinating agents that regulate obesity via the control of food intake mechanisms or the responses of receptor signaling pathways that regulate metabolism. Identification of deubiquitinating agents or negative effectors of deubiquitinating agents that regulate or inhibit the responses of neuropeptide Y (NPY), cholecystokinin and galanin receptors, are particularly desirable. Candidate libraries can be inserted into cells that have these receptors cloned into them, and modulatory molecules selected.

In a preferred embodiment, the present methods are useful in neurobiology applications. Deubiquitinating agents, negative effectors of deubiquitinating agents, or libraries thereof, may be used for screening for modulators of neuronal apoptotis, with an eye to preserving neuronal function and preventing of neuronal death. Initial screens would be done in cell culture. One application would include determining modulation of neuronal death, by apoptosis, in cerebral ischemia resulting from stroke. Apoptosis is known to be blocked by neuronal apoptosis inhibitory protein (NAIP); screens for its upregulation, down regulation, or affecting any coupled step could identify molecules which selectively modulate neuronal apoptosis. Other applications include neurodegenerative diseases such as Alzheimer's disease and Huntington's disease.

In a preferred embodiment, the present methods are useful in bone biology applications. Osteoclasts are known to play a key role in bone remodeling by breaking down “old” bone, so that osteoblasts can lay down “new” bone. In osteoporosis one has an imbalance of this process. Osteoclast overactivity can be regulated by inserting deubiquitinating agents, negative effectors of deubiquitinating agents, or libraries thereof, into these cells, and then looking for molecules that result in: 1) altrered processing of collagen by these cells; 2) altered pit formation on bone chips; and 3) altered release of calcium from bone fragments.

The present methods may also be used to screen for agonists of bone morphogenic proteins, hormone mimetics to stimulate, regulate, or enhance new bone formation (in a manner similar to parathyroid hormone and calcitonin, for example). These have use in osteoporosis, for poorly healing fractures, and to accelerate the rate of healing of new fractures. Furthermore, cell lines of connective tissue origin can be treated with deubiquitinating agents, negative effectors of deubiquitinating agents, or libraries thereof, and screened for their growth, proliferation, collagen stimulating activity, and/or proline incorporating ability on the target osteoblasts. Alternatively, deubiquitinating agents, negative effectors of deubiquitinating agents, or libraries thereof, can be expressed directly in osteoblasts or chondrocytes and screened for modulation of production of collagen or bone.

In a preferred embodiment, the present methods are useful in skin biology applications. Keratinocyte responses to a variety of stimuli may result in psoriasis, a proliferative change in these cells. Deubiquitinating agents, negative effectors of deubiquitinating agents, or libraries thereof, can be inserted into cells removed from active psoriatic plaques, and candidate deubiquitinating agents or dominant negative deubiquitinating agents isolated which modulate the rate of growth of these cells.

In a preferred embodiment, the present methods are useful in the identification of modulators of regulation of keloid formation (i.e. excessive scarring). Deubiquitinating agents, negative effectors of deubiquitinating agents, or libraries thereof, inserted into skin connective tissue cells isolated from individuals with this condition, and eubiquitinating agents can identify deubiquitinating agents that modulate proliferation, collagen formation, or proline incorporation. Results from this work can be extended to treat the excessive scarring that also occurs in burn patients. If a common modulator is found in the context of the keloid work, then it can be used widely in a topical manner to diminish scarring post burn.

Similarly, wound healing for diabetic ulcers and other chronic “failure to heal” conditions in the skin and extremities can be regulated by providing additional growth signals to cells which populate the skin and dermal layers. Growth factor mimetics may in fact be very useful for this condition. Deubiquitinating agents, negative effectors of deubiquitinating agents, or libraries thereof, can be inserted into skin connective tissue cells, and deubiquitinating agents identified which modulate the growth of these cells under “harsh” conditions, such as low oxygen tension, low pH, and the presence of inflammatory mediators.

Cosmeceutical applications of the present invention include the control of melanin production in skin melanocytes. A naturally occurring peptide, arbutin, is a tyrosine hydroxylase inhibitor, a key enzyme in the synthesis of melanin. Deubiquitinating agents, negative effectors of deubiquitinating agents, or libraries thereof, can be inserted into melanocytes and known stimuli that increase the synthesis of melanin applied to the cells. Candidate deubiquitinating agents can be identified that modulate the synthesis of melanin under these conditions.

In a preferred embodiment, the present methods are useful in endocrinology applications. The delivery methods desdcribed herein can be applied broadly to any endocrine, growth factor, cytokine or chemokine network which involves a signaling peptide or protein that acts in either an endocrine, paracrine or autocrine manner that binds or dimerizes a receptor and activates a signaling cascade that results in a known phenotypic or functional outcome. The methods are applied so as to identify a deubiquitinating agent that modulates the desired hormone (i.e., insulin, leptin, calcitonin, PDGF, EGF, EPO, GMCSF, IL1-17, mimetics) or or its action by either modulating the release of the hormone, modulating its binding to a specific receptor or carrier protein (for example, CRF binding protein), or modualting the intracellular responses of the specific target cells to that hormone. Identification of deubiquitinating agents which modulate the expression or release of hormones from the cells which normally produce them could have broad applications to conditions of hormonal deficiency.

In a preferred embodiment, the present methods are useful in infectious disease applications. Viral latency (herpes viruses such as CMV, EBV, HBV, and other viruses such as HIV) and their reactivation are a significant problem, particularly in immunosuppressed patients (patients with AIDS and transplant patients). The ability to block the reactivation and spread of these viruses is an important goal. Cell lines known to harbor or be susceptible to latent viral infection can be infected with the specific virus, and then stimuli applied to these cells which have been shown to lead to reactivation and viral replication. This can be followed by measuring viral titers in the medium and scoring cells for phenotypic changes. Deubiquitinating agents, negative effectors of deubiquitinating agents, or libraries thereof, can then be introduced into these cells under the above conditions, and agents identified which modulate the growth and/or release of the virus. As with chemotherapeutics, these experiments can also be done with drugs which are only partially effective towards this outcome, and bioactive peptides isolated which enhance the virucidal effect of these drugs. Agents may also be tested for the ability to block some aspect of viral assembly, viral replication, entry or infectious cycle. Additional disclosure directed to reduction of viral infection, including HIV, is set forth in 09/800,770, filed Mar. 6, 2001, which is expressly incorporated herein by reference.

In a preferred embodiment, the present invention finds use with infectious organisms. Intracellular organisms such as mycobacteria, listeria, salmonella, pneumocystis, yersinia, leishmania, T. cruzi, can persist and replicate within cells, and become active in immunosuppressed patients. There are currently drugs on the market and in development which are either only partially effective or ineffective against these organisms. Deubiquitinating agents, negative effectors of deubiquitinating agents, or libraries thereof, can be inserted into specific cells infected with these organisms (pre- or post-infection), and deubiquitinatinmg agents identified which modulate the intracellular destruction of these organisms in a manner analogous to intracellular “antibiotic peptides” similar to magainins. In addition deubiquitinating agents can be identified which modulate the cidal properties of drugs already under investigation which have insufficient potency by themselves, but when combined with a specific peptide from a candidate library, are dramatically more potent through a synergistic mechanism. Finally, deubiquitinating agents can be identified which affect the metabolism of these intracellular organisms, with an eye towards terminating their intracellular life cycle by inhibiting a key organismal event.

Antibiotic drugs that are widely used have certain dose dependent, tissue specific toxicities. For example renal toxicity is seen with the use of gentamicin, tobramycin, and amphotericin; hepatotoxicity is seen with the use of INH and rifampin; bone marrow toxicity is seen with chloramphenicol; and platelet toxicity is seen with ticarcillin, etc. These toxicities limit their use. deubiquitinating agents, negative effectors of deubiquitinating agents, or libraries thereof, can be introduced into the specific cell types where specific changes leading to cellular damage or apoptosis by the antibiotics are produced, and deubiquitinating agents can be identified that modulate sensitivity, when these cells are treated with these specific antibiotics.

Furthermore, the present invention finds use in screening for deubiquitinating agents that modulate antibiotic transport mechanisms. The rapid secretion from the blood stream of certain antibiotics limits their usefulness. For example penicillins are rapidly secreted by certain transport mechanisms in the kidney and choroid plexus in the brain. Probenecid is known to block this transport and increase serum and tissue levels. Deubiquitinating agents, negative effectors of deubiquitinating agents, or libraries thereof, can be inserted into specific cells derived from kidney cells and cells of the choroid plexus known to have active transport mechanisms for antibiotics. Deubiquitinating agents can then be identified which block the active transport of specific antibiotics and thus extend the serum halflife of these drugs.

In a preferred embodiment, the present methods are useful in drug toxicities and drug resistance applications. Drug toxicity is a significant clinical problem. This may manifest itself as specific tissue or cell damage with the result that the drug's effectiveness is limited. Examples include myeloablation in high dose cancer chemotherapy, damage to epithelial cells lining the airway and gut, and hair loss. Specific examples include adriamycin induced cardiomyocyte death, cisplatinin-induced kidney toxicity, vincristine-induced gut motility disorders, and cyclosporin-induced kidney damage. Deubiquitinating agents, negative effectors of deubiquitinating agents, or libraries thereof, can be introduced into specific cell types with characteristic drug-induced phenotypic or functional responses, in the presence of the drugs, and deubiquitinating agents identified which modulate toxicity in the specific cell type when exposed to the drug. These effects may manifest as modulating the drug induced apoptosis of the cell of interest, thus initial screens will determine relative survival of the cells in the presence of high levels of drugs or combinations of drugs used in combination chemotherapy.

Drug toxicity may be due to a specific metabolite produced in the liver or kidney which is highly toxic to specific cells, or due to drug interactions in the liver which block or enhance the metabolism of an administered drug. Deubiquitinating agents, negative effectors of deubiquitinating agents, or libraries thereof, can be introduced into liver or kidney cells following the exposure of these cells to the drug known to produce the toxic metabolite. Deubiquitinating agents can be identified which alter how the liver or kidney cells metabolize the drug, and specific deubiquitinating agents identified which modulate the generation of a specific toxic metabolite. The generation of the metabolite can be followed by mass spectrometry, and phenotypic changes can be assessed by microscopy. Such a screen can also be done in cultured hepatocytes, cocultured with readout cells which are specifically sensitive to the toxic metabolite. Applications include reversible (to limit toxicity) inhibitors of enzymes involved in drug metabolism.

Multiple drug resistance, and hence tumor cell selection, outgrowth, and relapse, leads to morbidity and mortality in cancer patients. Deubiquitinating agents, negative effectors of deubiquitinating agents, or libraries thereof, can be introduced into tumor cell lines (primary and cultured) that have demonstrated specific or multiple drug resistance. Deubiquitinating agents can then be identified which modulate drug sensitivity when the cells are exposed to the drug of interest, or to drugs used in combination chemotherapy. The readout can be the onset of apoptosis in these cells, membrane permeability changes, the release of intracellular ions and fluorescent markers. The cells in which multidrug resistance involves membrane transporters can be preloaded with fluorescent transporter substrates, and selection carried out for deubiquitinating agents which modulate the normal efflux of fluorescent drug from these cells.

Deubiquitinating agents, negative effectors of deubiquitinating agents, and in particular libraries thereof, are suited to screening for deubiquitinating agents which modulate poorly characterized or recently discovered intracellular mechanisms of resistance or mechanisms for which few or no chemosensitizers currently exist, such as mechanisms involving LRP (lung resistance protein). This protein has been implicated in multidrug resistance in ovarian carcinoma, metastatic malignant melanoma, and acute myeloid leukemia. Particularly interesting examples include screening for deubiquitinating agents which modulate more than one important resistance mechanism in a single cell, which occurs in a subset of the most drug resistant cells, which are also important targets. Applications would include screening for deubiquitinating agent modulators of both MRP (multidrug resistance related protein) and LRP for treatment of resistant cells in metastatic melanoma, for modulators of both p-glycoprotein and LRP in acute myeloid leukemia, and for modulation (by any mechanism) of all three proteins for treating pan-resistant cells.

In a preferred embodiment, the present methods are useful in improving the performance of existing or developmental drugs. First pass metabolism of orally administered drugs limits their oral bioavailability, and can result in diminished efficacy as well as the need to administer more drug for a desired effect. Reversible inhibitors of enzymes involved in first pass metabolism may thus be a useful adjunct enhancing the efficacy of these drugs. First pass metabolism occurs in the liver, thus inhibitors of the corresponding catabolic enzymes may enhance the effect of the cognate drugs. Reversible inhibitors would be delivered at the same time as, or slightly before, the drug of interest. Screening of deubiquitinating agents, negative effectors of deubiquitinating agents, or libraries thereof, in hepatocytes for modulators (by any mechanism, such as protein downregulation as well as a direct inhibition of activity) of particularly problematical isozymes would be of interest. These include the CYP3A4 isozymes of cytochrome P450, which are involved in the first pass metabolism of the anti-HIV drugs saquinavir and indinavir. Other applications could include reversible inhibitors of UDPglucuronyltransferases, sulfotransferases, N-acetyltransferases, epoxide hydrolases, and glutathione S-transferases, depending on the drug. Screens would be done in cultured hepatocytes or liver microsomes, and could involve antibodies recognizing the specific modification performed in the liver, or co-cultured readout cells, if the metabolite had a different bioactivity than the untransformed drug. The enzymes modifying the drug would not necessarily have to be known, if screening was for lack of alteration of the drug.

In a preferred embodiment, the present methods are useful in immunobiology, inflammation, and allergic response applications. Selective regulation of T lymphocyte responses is a desired goal in order to modulate immune-mediated diseases in a specific manner. Deubiquitinating agents, negative effectors of deubiquitinating agents, or libraries thereof, can be introduced into specific T cell subsets (TH1, TH2, CD4+, CD8+, and others) and the responses which characterize those subsets (cytokine generation, cytotoxicity, proliferation in response to antigen being presented by a mononuclear leukocyte, and others) modified by members of the library. Deubiquitinating agents can be identified which modulate the known T cell subset physiologic response. This approach will be useful in any number of conditions, including: 1) autoimmune diseases where one wants to induce a tolerant state (select a peptide that inhibits T cell subset from recognizing a self-antigen bearing cell); 2) allergic diseases where one wants to decrease the stimulation of IgE producing cells (select peptide which blocks release from T cell subsets of specific B-cell stimulating cytokines which induce switch to IgE production); 3) in transplant patients where one wants to induce selective immunosuppression (select peptide that diminishes proliferative responses of host T cells to foreign antigens); 4) in lymphoproliferative states where one wants to inhibit the growth or sensitize a specific T cell tumor to chemotherapy and/or radiation; 5) in tumor surveillance where one wants to inhibit the killing of cytotoxic T cells by Fas ligand bearing tumor cells; and 5) in T cell mediated inflammatory diseases such as Rheumatoid arthritis, Connective tissue diseases (SLE), Multiple sclerosis, and inflammatory bowel disease, where one wants to inhibit the proliferation of disease-causing T cells (promote their selective apoptosis) and the resulting selective destruction of target tissues (cartilage, connective tissue, oligodendrocytes, gut endothelial cells, respectively).

Regulation of B cell responses will permit a more selective modulation of the type and amount of immunoglobulin made and secreted by specific B cell subsets. Deubiquitinating agents, negative effectors of deubiquitinating agents, or libraries thereof, can be inserted into B cells and deubiquitinating agents identified which modulate the release and synthesis of a specific immunoglobulin. This may be useful in autoimmune diseases characterized by the overproduction of auto antibodies and the production of allergy causing antibodies, such as IgE. Deubiquitinating agents can also be identified which inhibit or enhance the binding of a specific immunoglobulin subclass to a specific antigen either foreign of self. Finally, deubiquitinating agents can be identified which inhibit the binding of a specific immunoglobulin subclass to its receptor on specific cell types.

Similarly, deubiquitinating agents which affect cytokine production may be identified, generally using two cell systems. For example, cytokine production from macrophages, monocytes, etc. may be evaluated. Similarly, deubiquitiniating agents which modulate cytokines, for example erythropoetin and IL1-17, may be identified.

Antigen processing by mononuclear leukocytes (ML) is an important early step in the immune system's ability to recognize and eliminate foreign proteins. Deubiquitinating agents, negative effectors of deubiquitinating agents, or libraries thereof, can be inserted into ML cell lines and agents selected which alter the intracellular processing of foreign peptides and sequence of the foreign peptide that is presented to T cells by MLs on their cell surface in the context of Class II MHC. One can look for dubiquitinating agents, negative effectors of deubiquitinating agents, or libraries thereof, that affect responses of a particular T cell subset (for example, the peptide would in fact work as a vaccine). This approach could be used in transplantation, autoimmune diseases, and allergic diseases.

The release of inflammatory mediators (cytokines, leukotrienes, prostaglandins, platelet activating factor, histamine, neuropeptides, and other peptide and lipid mediators) is a key element in maintaining and amplifying aberrant immune responses. Deubiquitinating agents, negative effectors of deubiquitinating agents, or libraries thereof, can be inserted into MLs, mast cells, eosinophils, and other cells participating in a specific inflammatory response, and deubiquitinating agents identifies that modulate the release and binding to the cognate receptor of each of these types of mediators.

In one embodiment wherein a libray is screened, the method further comprises isolating at least one altered cell with said altered phenotype. Methods of isolating cells are known in the art and include, but are not limited to, FACS analysis and isolation, growth on selective medium, clonal isolation of cells and the like. In general, once the cell with the altered phenotype is identified, the cell(s) is then isolated for further analysis, e.g. to determine which deubiquitinating agent variant resulted in the altered phenotype.

Accordingly, the method further comprises identifying said variant agent in said altered cell. That is, once the cell(s) with the altered phenotype is identified and isolated, the nucleic acid encoding the deubiquitinating agents or negative effector of a deubiquitinating agent is identified. This is accomplished by isolating from the cellular DNA the insert encoding the deubiquitinating agent variant. Preferably this is performed by PCR.

It is understood by the skilled artisan that the steps of the assays provided herein can vary in order. It is also understood, however, that while various options (of compounds, properties selected or order of steps) are provided herein, the options are also each provided individually, and can each be individually segregated from the other options provided herein. Moreover, steps which are obvious and known in the art that will increase the sensitivity of the assay are intended to be within the scope of this invention. For example, there may be additionally washing steps, blocking steps, etc.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are expressly incorporated by reference in their entirety.

### EXAMPLES

In the first four Examples that follow, the deubiquitinating activity of the deubiquitinating agent UbpM was assayed for using different ubiquitin complexes as described below and two different tagged UbpM constructs, Gst-UbpM and His-UbpM. The full-length UbpM was used to construct Gst-UbpM, and His-UbpM. In Gst-UbpM. the Gst tag is located at the amino terminus of the full length UbpM, and in His UbpM, the His tag is located at the amino terminus of the full length UbpM. Gst-UbpM was expressed in E. coli cells and His-UbpM was expressed in Sf9 cells.

The deubiquitinating activity of Gst-UbpM and His-UbpM, was detected using the following reaction buffer: 50 mM Hepes pH7.6, 0.5 mM EDTA, 0.1 mg/ml BSA, lmM DTT.

### Example 1

### Assayinq for UbDM Deubiguitinating Activity Using Ubiquitin-AMC as a Ubiguitin Complex.

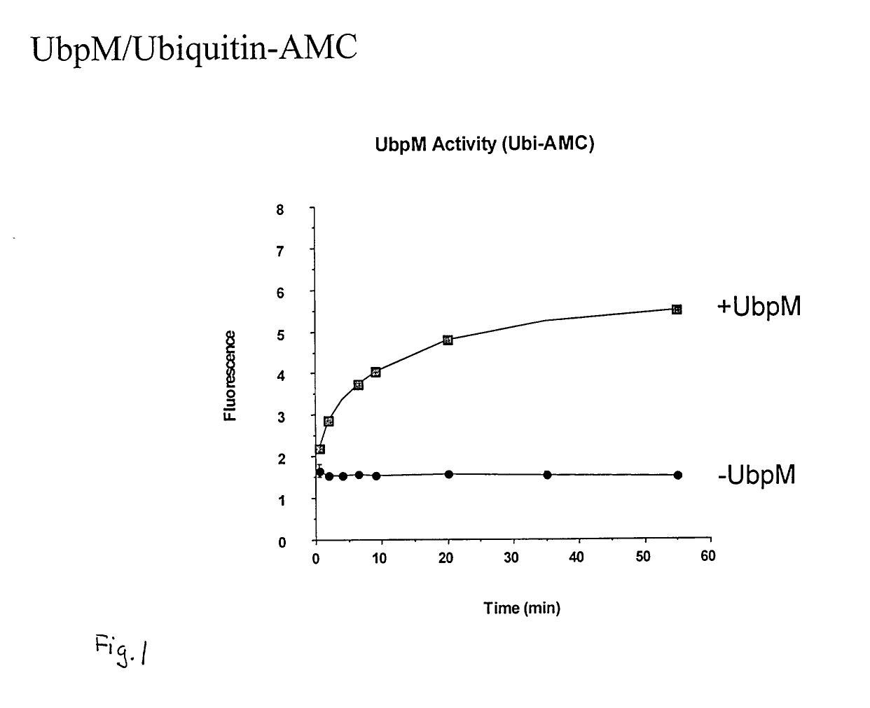

In separate reaction mixtures, Gst-UbpM and His-UbpM were each incubated with the fluorogenic substrate of Ubiquitin-AMC (7-amido-4-methylcoumarin) at room temperature up to 1 hour. The results indicate that Ub-AMC cleavable ubiquitin fusion polypeptide was hydrolyzed with release of highly fluorescent AMC, which was detected by a fluoro-scanner (see FIG. 1).

### Example 2

### Assaving for UbpM Deubiquitinating Activity Using Purified Poly-Ubiguitin2-7 Protein as a Ubiguitin Complex.

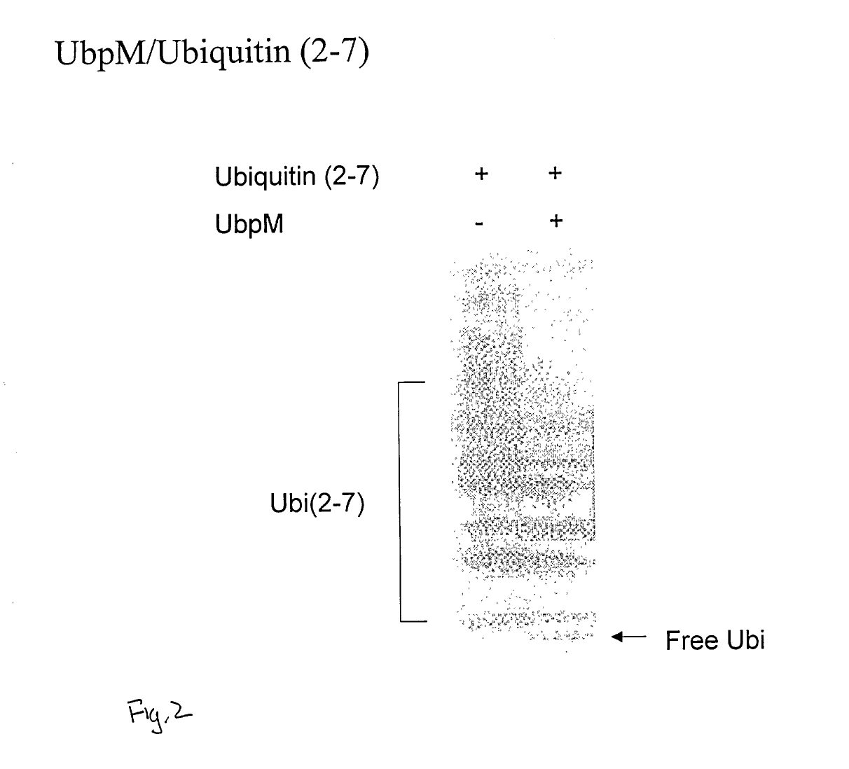

Purified poly-Ubiquitin 2-7 cleavable ubiquitin fusion polypeptide was purchased from commercial source. In separate reaction mixtures, Gst-UbpM and His-UbpM were each incubated with Ubi2-7 for 1 hr at room temperature. The reaction was stopped and then a Western blot was performed on the products of the reaction using anti-ubiquitin antibody. Results of the Western blot analysis show a reduction of the polyubiquitin ladders of the ubiquitin complex, and appearance of free ubiquitin moiety, indicative of deubiquitination (see FIG. 2).

### Example 3

### Assaying for UbpM Deubiguitinating Activity Using Linear Flag-Ubiquitin-His(6) Fusion Proteins as a Ubiguitin complex.

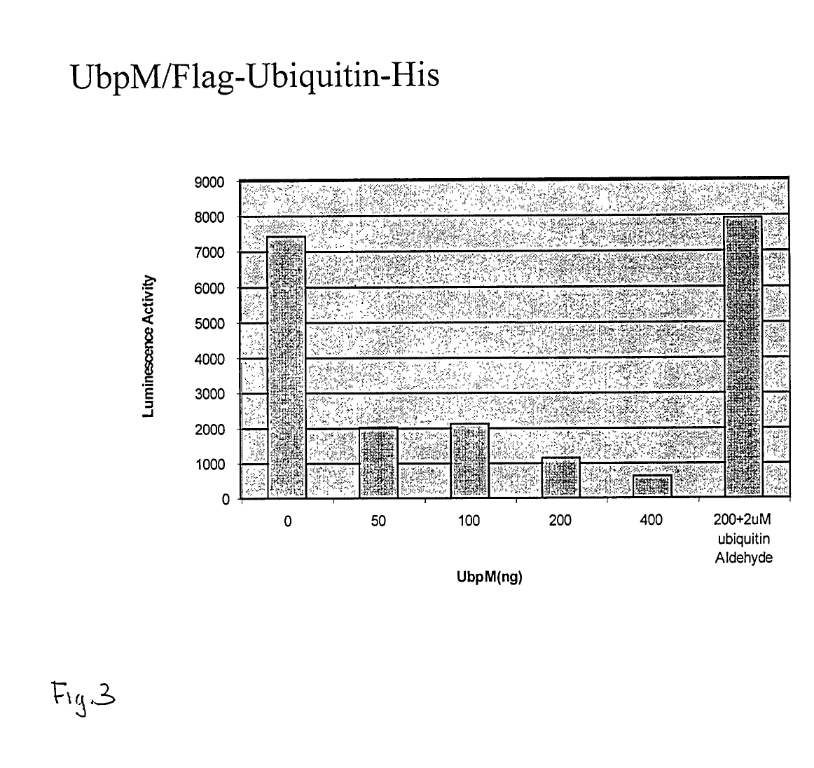

Flag-ubiquitin-His(6) cleavable ubiquitin fusion polypeptide was expressed in E. coli and then purified. In separate reaction mixtures, Gst-UbpM and His-UbpM were each incubated with purified Flagubiquitin-His in a 96 well Ni-plate for 1 hour at room temperature. The plate was then washed. Flagubiquitin moiety cleaved by Gst-UbpM or His-UbpM was released from the plate leaving only His(6) attached to the plate. Any uncleaved Flag-ubiquitin-His attached to the Ni-plate was then detected by anti-Flag ELISA (see FIG. 3).

### Example 4

### Assaying for UbpM Deubiquitinating Activity Using Auto-Ubiquitinated Ligase Containing Poly-Ubiquitin Chain as a Ubiguitin Complex.

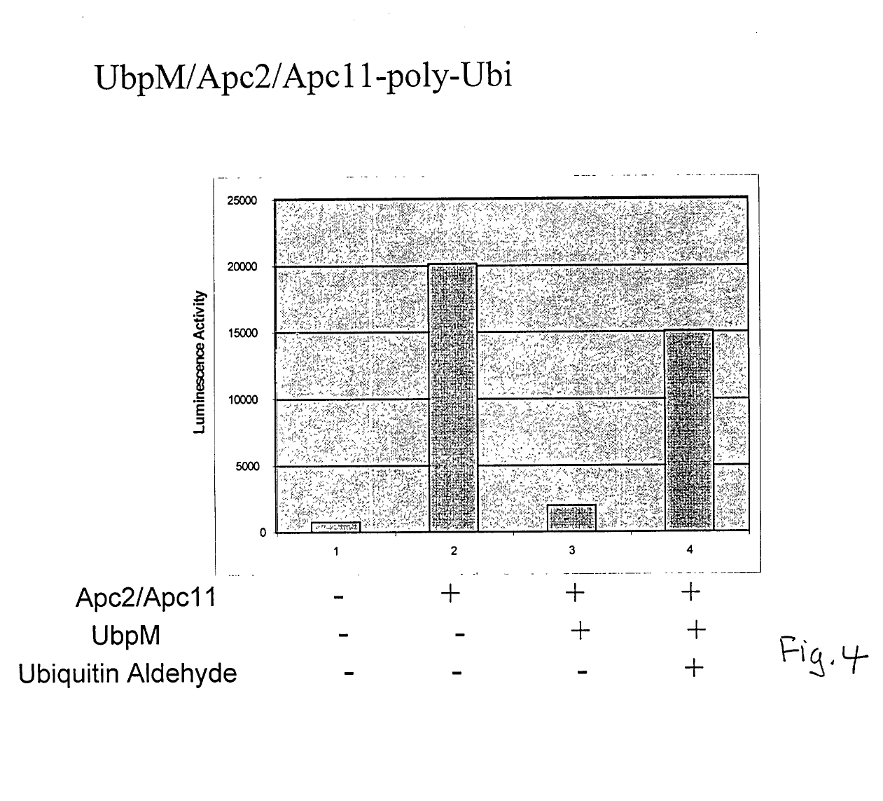

E1, E2, and His-E3 ubiquitin agents were combined in vitro in a reaction mixture to form the auto-ubiquitinated E3 ubiquitin complex attached to a Ni-plate. The plate was then washed to remove any free ubiquitin or unbound poly-ubiquitin chains. In separate reaction mixtures, Gst-UbpM and His-UbpM, were each combined with the auto-ubiquitinated E3 ubiquitin complex attached to a Ni-plate for an hour at room temperature for the deubiquitination reaction. Free Flag-ubiquitin moiety resulting from deubiquitination of the poly-ubiquitin chain of the ubiquitin complex was then washed away, and any remaining poly-Flag-ubiquitin moiety attached to the Ni-plate was detected by an anti-Flag immunoassay (see FIG. 4).

### Example 5

### Assaving for UbpM Deubiquitinating Activity Using FRET-Pair Labeled Poly-Ubiguitin Chain

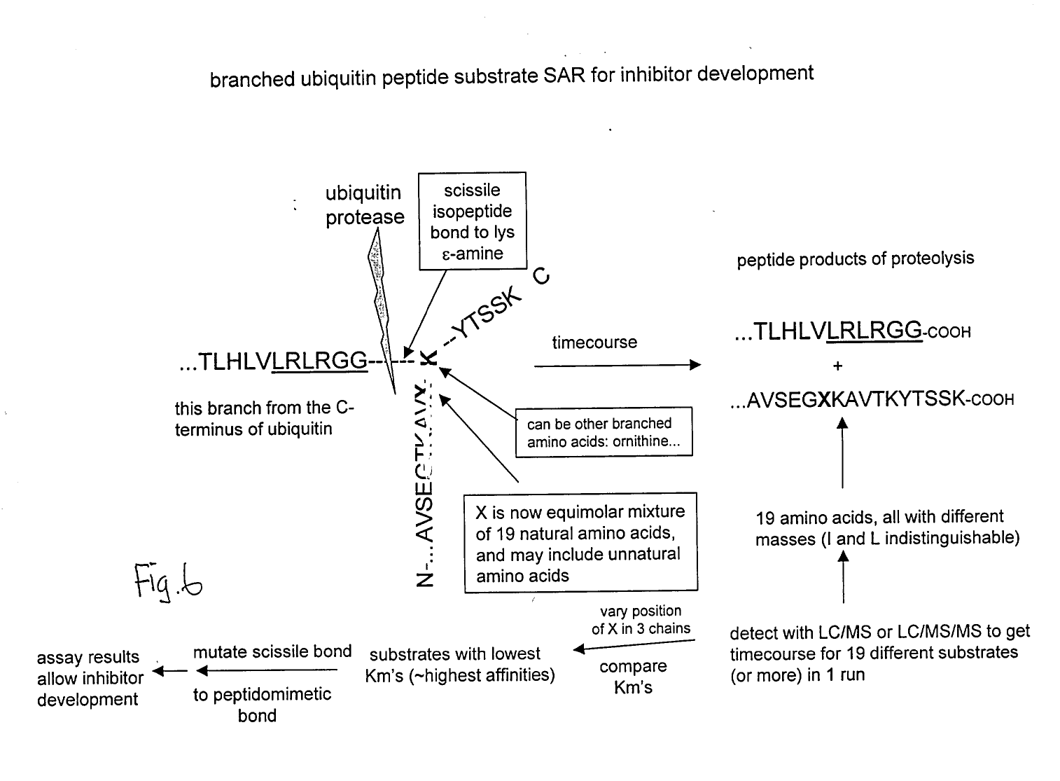

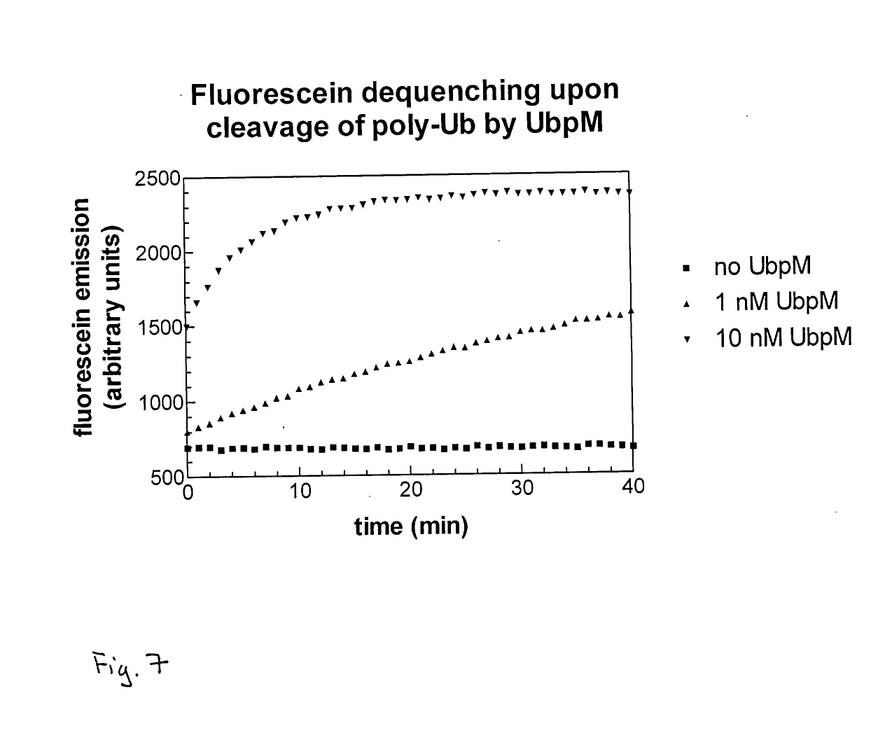

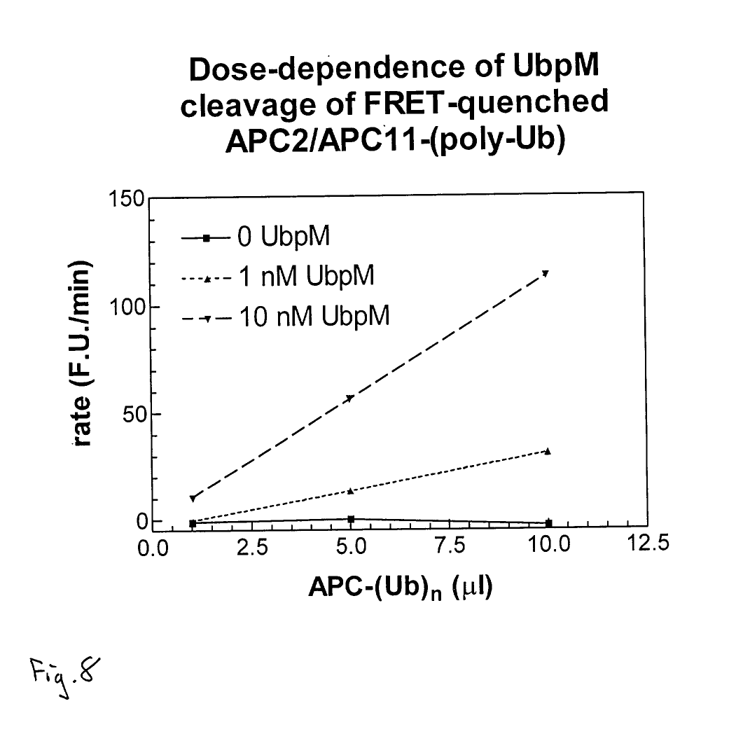

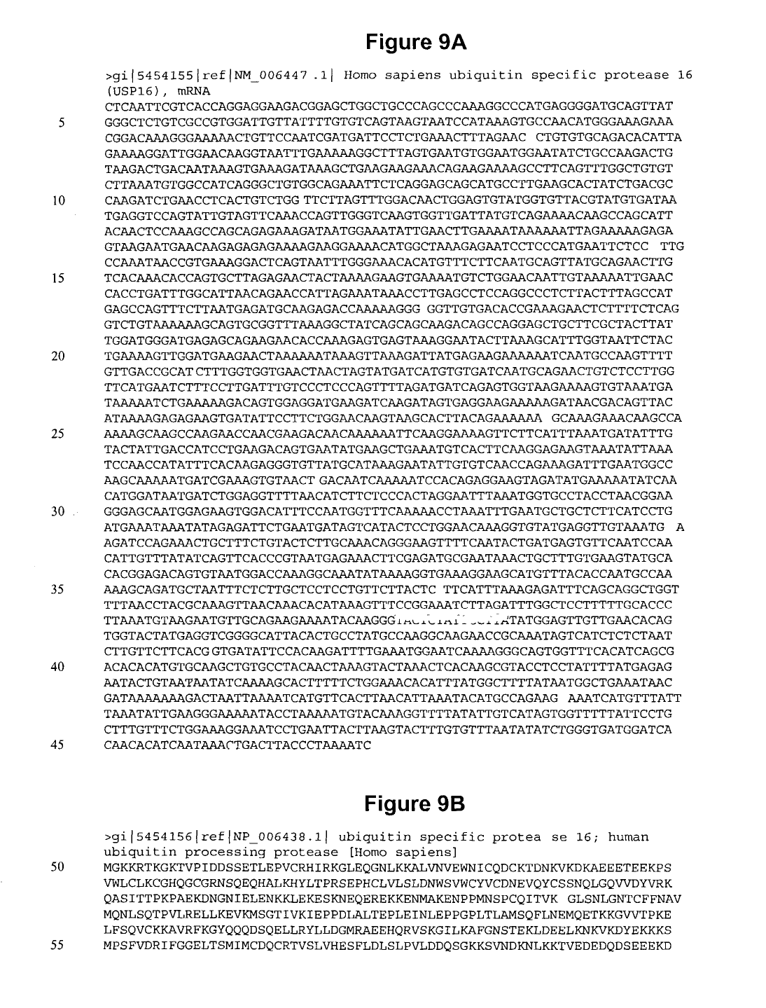

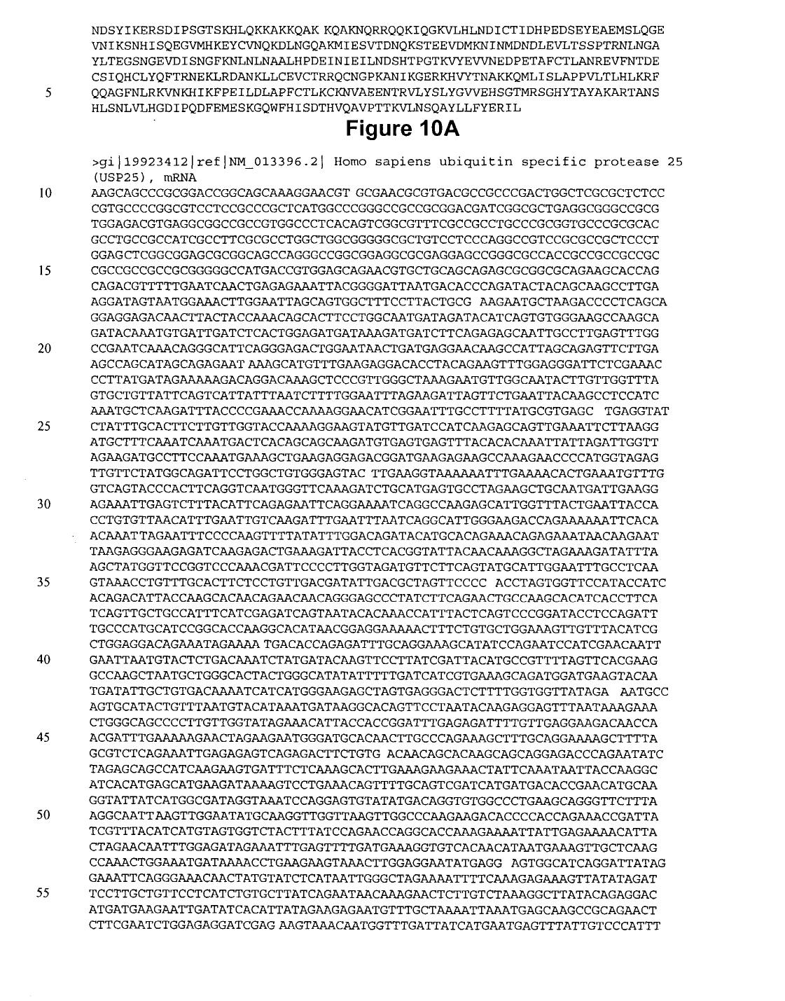

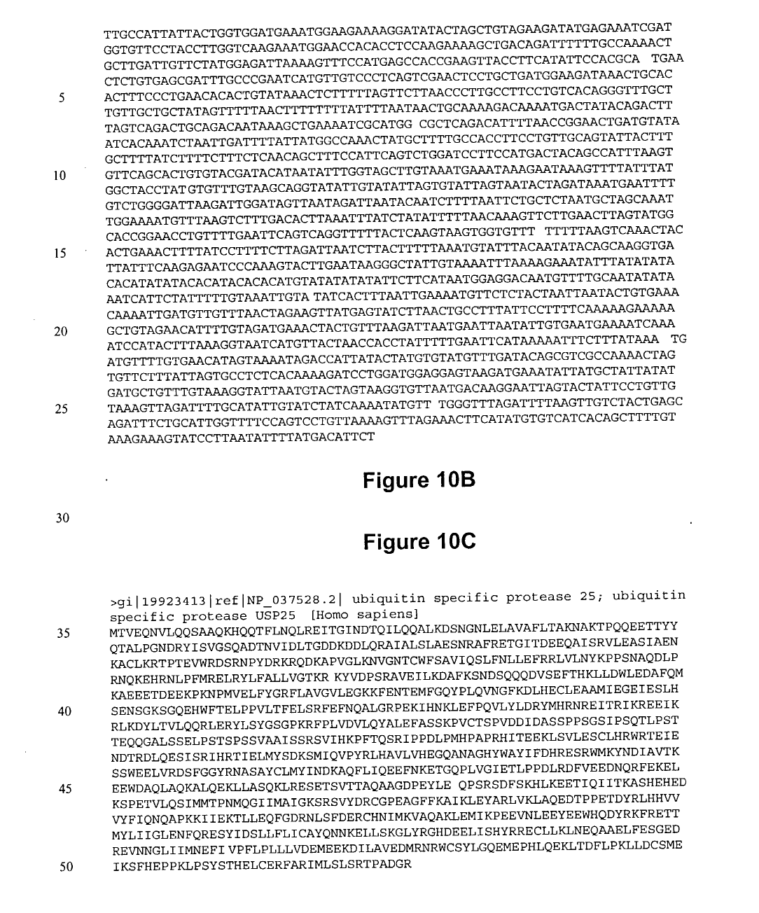

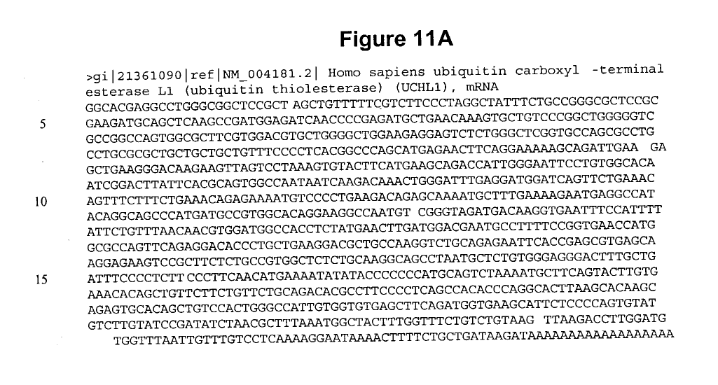

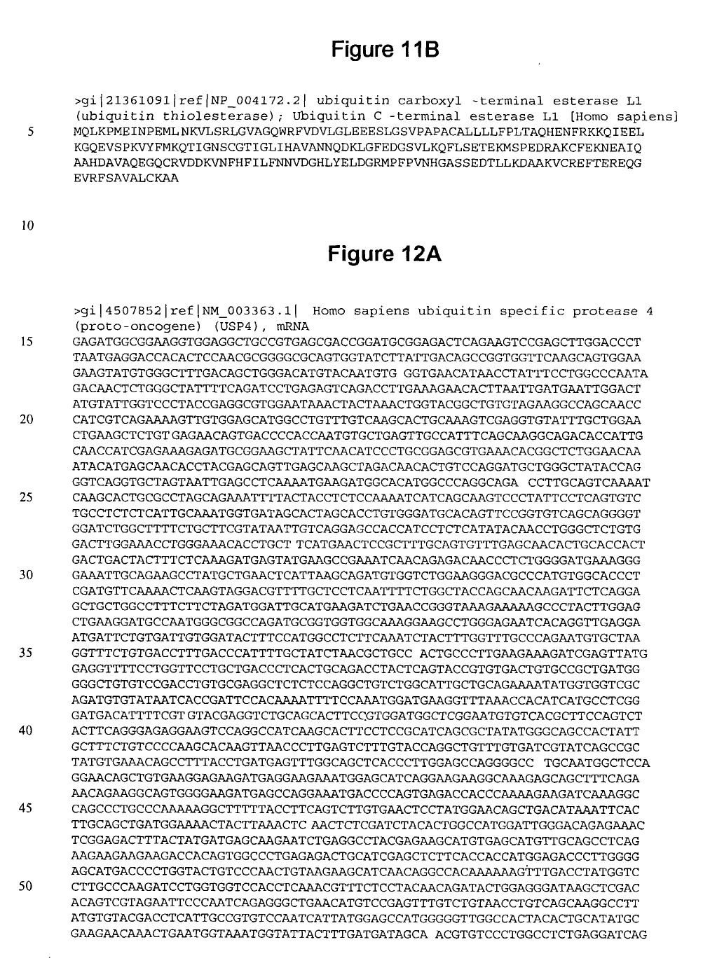

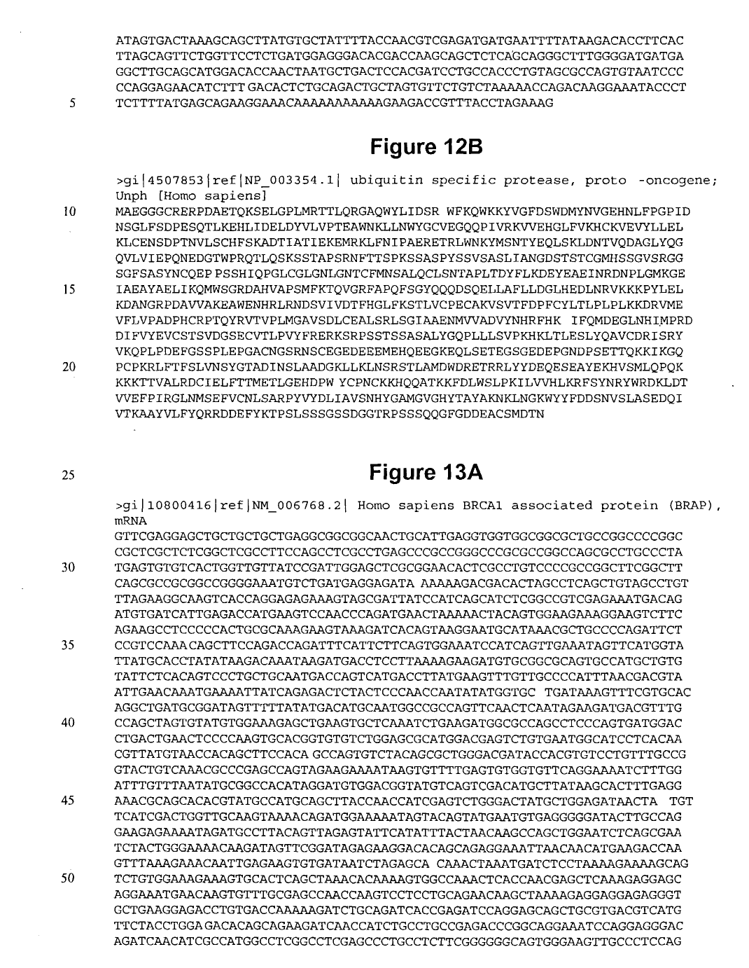

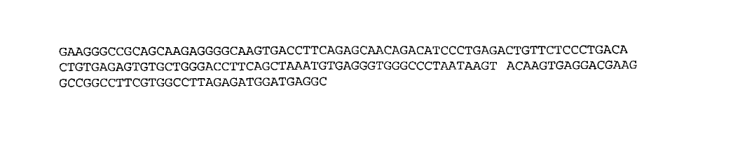

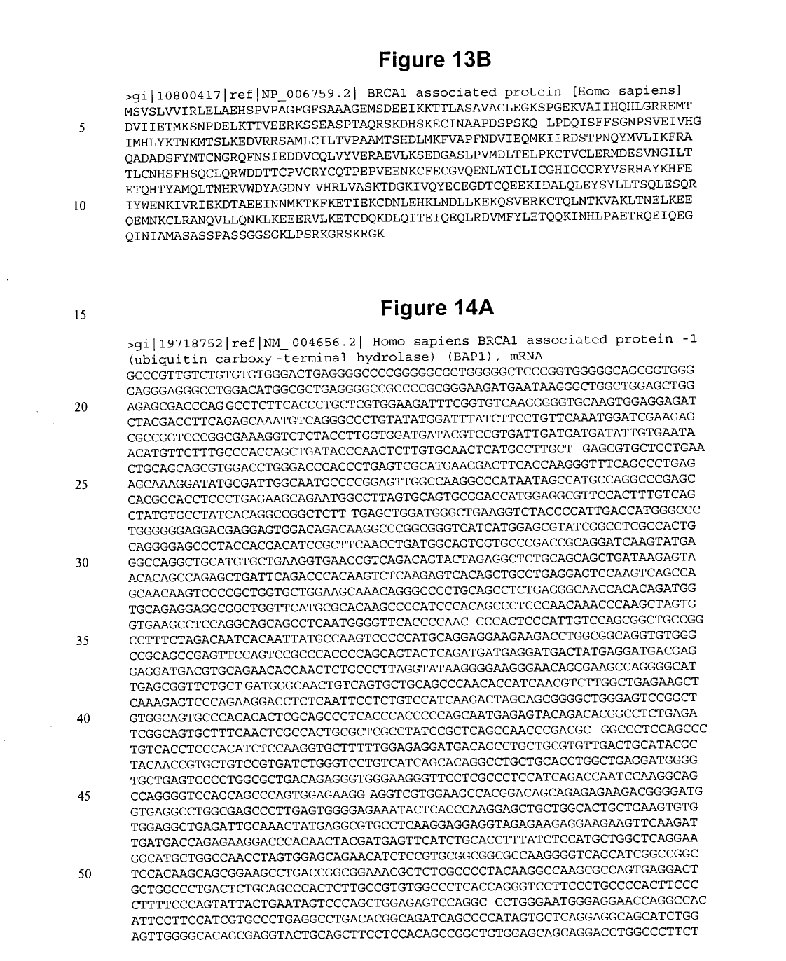

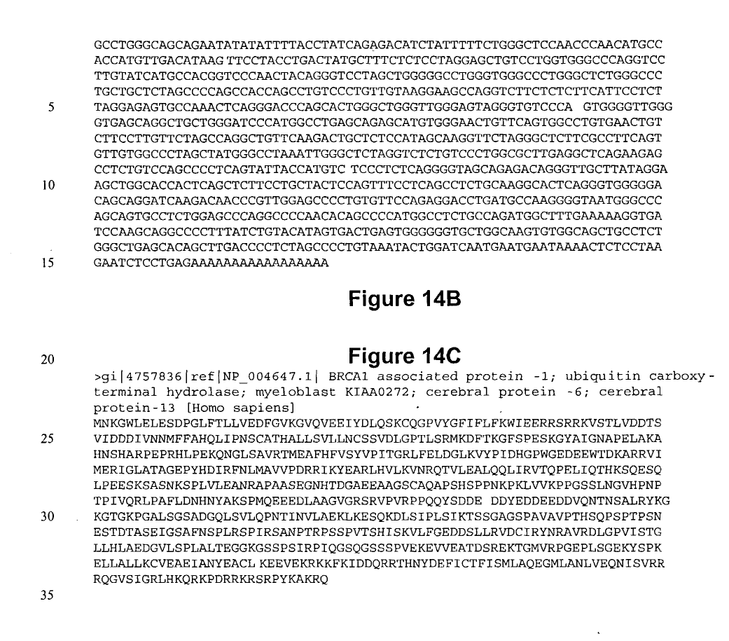

The following reaction was carried out in a buffer containing 50 mM Tris buffer (pH 8), 0.5 mM EDTA and 1 mM TCEP. FLAG-Cys(fluorescein)-Ub, FLAG-Cys(TAMRA)-Ub, E1, E2, and His-E3 agents were combined in vitro in a reaction mixture to form the auto-ubiquitinated E3 complex in solution, rather than attached to a Ni-plate as in example 4. This E3-Ub complex contains equal proportions of the FRET pair fluorophores fluorescein and TAMRA linked via a Cys residue between the FLAG tag and Ub moiety; TAMRA strongly quenches fluorescein emission from these Ub polymers. An aliquot of this reaction product was then incubated with His-UbpM at the concentrations noted in FIGS. 7 and 8, and the reaction progress was continuously monitored by exciting at 488 nm and detecting at 520 nm on a fluorescence microplate reader. The time-dependent and dose-dependent fluorescence enhancement correlates with the deubiquitinating activity of UbpM, and results from the spatial separation of Cys(fluorescein)-Ub from Cys(TAMRA)-Ub upon proteolytic cleavage.

### Example6

### A549, HUVEC, HBEC ICAM (CD54) Induction Assay

The ICAM upregulation assay models the inflammatory process and cytokine signaling. ICAM is an adhesion molecule that is expressed on the surface of cells at local sites of inflammation. ICAM expression is induced in the presence of various cytokines such as IL-1β, TNFα, and IFNγ. Each cytokine acts through different signaling molecules therefore this assay can delineate the specificity of a particular genetic effector (i.e. siRNA or a dominant interfering mutant) (see FIG. 2).

Day1:

Split cells (A549, HBEC, or HUVEC) cells 4.5×10⁴ in a 24 well plate in the appropriate media and incubate at 37□C., 5% C02.

Day 2: Cells Should be 40-50% Confluent

siRNA

Transfect siRNA with oligofectamine. Pipette out the media and replace it with 500 uL of fresh media. Mix 3 uL of 20 uM siRNA duplexes with 50 uL of Optimem media. Add 3 uL of oligofectamine to 12 uL Optimen. Wait 7-10 minutes. Combine the two solutions and gently pipette up and down 3 times. Wait 20-25 minutes. Add 32 uL of Optimen to adjust the volume to 100 uL. Add the entire mixture to the cells.

Retroviral

Infect cells using a standard spin infection protocol.

Day 3: Add 0.5 mL of Fresh Media

Day 4:

Wash cells in 1 mL PBS, remove PBS and add 100 uL of Trypsin/EDTA. 5 min later add 100 uL of FK12. Pipette 4× up and down then transfer the cells to a V-bottom 96 well plate. Spin down at 1200 rpm for 3 min. Resuspend in 200 uL of fresh media. Count representative wells by hemocytometer then compute the average cells/ml. Plate 1.5×10⁴ cells/well in a 96 well plate, the total final volume is 50 uL.

Day 5:

Add 50 uL of a 2× cytokine mixture; the final concentrations of recombinant IL-1α, TNFβ, and IFNγ should be 75 ng/mL. All cytokines can be purchased from Peprotech as a lyophilized powder.

Day 6: Stain Cells and FACs Analysis

Rinse the cells 1×200 uL PBS. Add 50 uL of Trypsin/EDTA-lncubate 5 min at 37□C. Add 150 uL of PBS-2% FCS B Pipette up and down 5× and transfer to a V-bottom 96 well plate. Spin down and wash 1× in 200 uL PBS-EDTA, remove solution. Add 25 uL of a 1:7 dilution of ICAM-APC (Pharmingen). Pipette up and down gently 4x to resuspend the cells. Incubate in the dark for 15 min at 4□C. Add 175 uL of PBS-2%FCS. Spin down at 2000 rpm for 30 sec. Wash once with 200 uL PBS-2%FCS. Add 150 uL of PBS-2%, resuspend the cells, then transfer to cluster tubes.

Perform FACS analysis on FL4-APC for siRNA analysis, FL4-APC vs. FL1-GFP for retroviral IRES or GFP-fusion analysis.

### Example 7

Jurkat and BJAB Activation Protocols

T/B Cell CD69 assay: For CD69 upregulation experiments, tTA-BJAB or tTA-Jurkat cells are split to 2.5×10⁵ cells/ml 24 hours prior to stimulation. Cells are spun and resuspended at 5×10⁵ cells/ml in fresh complete RPMI medium in the presence of 0.3 ug/mI anti-lgM F(ab′)2 (Jackson Immunoresearch), 300 ng/ml C305 (anti-Jurkat clonotypic TCR (19)) or 5 ng/ml PMA for 16-20 hours at 371 C. Jurkat-N or tTA-BJAB cells are then stained with an APC-conjugated mouse monoclonal anti-human CD69 antibody (Caltag) at 41C for 30 minutes and analyzed using a Facscalibur instrument (Becton Dickinson) with Celiquest software. T cell CD28RE-RFP assay: tTA-Jurkat cells stably transfected with a CD28RE/AP-driven RFP construct are split to 2.5×10⁵ cells/ml 24 hours prior to stimulation. Cells are spun and resuspended at 5×10⁵ cells/ml in fresh complete RPMI medium in the presence of plate-coated 300 ng/ml C305 (anti-Jurkat clonotypic TCR (19)) plus 1 ug/ml a-CD28, or 5 ng/ml PMA plus 1 uM lonmycin for 16-20 hours at 371C. Jurkat-N cells are then analyzed using a Facscalibur instrument (Becton Dickinson) with Cellquest software (data not shown).

### Example 8

LDL-Receptor Upregulation

This assay measures cytokine induced LDL-Receptor expression on HepG2 cells. Similar to A549-ICAM screen, HepG2 cells can be infected with retroviral vectors or transfected with siRNA, stimulated with various cytokines, and LDL receptor can be measured with FACs or by an LDL-binding assay (J Biol Chem 1993 Aug 15;268(23):17489-94, which is expressly incorporated herein by reference).

Day1:

Split cells HepG2 cells 4.5×10⁴ in a 24 well plate in the appropriate media and incubate at 37□C., 5% C02.

Day 2: Cells Should be 40-50% Confluent

siRNA

Transfect siRNA with oligofectamine. Pipet out media and replace with 500 uL of fresh media. Mix 3uL of 20 uM siRNA duplexes with 50 uL of Optimem media. Add 3 uL of oligofectamine to 12 uL optimem. Wait 7-10 minutes. Combine the two solutions and pipet up and gently pipet up and down 3 times. Wait 20-25 minutes. Add 32 uL of optimem to adjust the volume to 100 uL. Add the entire mixture to the cells.

Retroviral

Infect cells using a standard spin infection protocol.

Day 3: Add 0.5 mL of Fresh Media

Day 4:

Wash cells in 1 mL PBS, remove PBS and add 100 uL of Trypsin/EDTA. 5 min later add 100 uL of fresh media. Pipet 4× up and down then transfer to a V-boftom 96 well plate. Spin down at 1200 rpm for 3 min. Resuspend in 200 uL of fresh media. Count representative wells by hemocytometer then compute the average cells/ml. Plate 1.5×10⁴ cells/well in a 96 well plate, the total final volume is 50 uL.

Day 5:

Add 50 uL of a 2× cytokine mixture. All cytokines can be purchased from Peprotech as a lyopholized powder.

Day 6: Detect LDL-Recptor Numbers with the LDL Binding Assay.

Rinse the cells 1×200 uL PBS and proceed with the binding assay as described previously (J Biol Chem Aug 15, 1993 ;268(23):17489-94).

### Example 9

### CHMC Low Cell Density IgE Activation: Tryptase and LTC4 Assays

Cultured human mast cells (CHMC) are obtained as described in U.S. Ser. No. 10/053,355, particularly at pages 46-50 which is expressly incorporated herein by reference. Screens for mast cell activation are performed as described below.

To duplicate 96-well U-bottom plates (Costar 3799) add 65 ul of compound dilutions or control samples that have been prepared in MT [137 mM NaCl, 2.7 mM KCl, 1.8 mM CaCl₂, 1.0 mM MgCl₂, 5.6 mM Glucose, 20 mM Hepes (pH 7.4), 0.1% Bovine Serum Albumin, (Sigma A4503)] containing 2% MeOH and 1% DMSO. Pellet CHMC cells (980 rpm, 10 min) and resuspend in pre-warmed MT. Add 65 ul of cells to each 96-well plate. Depending on the degranulation activity for each particular CHMC donor, load 1000-1500 cells/well. Mix four times followed by a 1 hr incubation at 37° C. During the 1 hr incubation, prepare 6× anti-IgE solution [rabbit anti-human IgE (1 mg/ml, Bethyl Laboratories A80-109A) diluted 1:167 in MT buffer]. Stimulate cells by adding 25 ul of 6× anti-IgE solution to the appropriate plates. Add 25 ul MT to un-stimulated control wells. Mix twice following addition of the anti-igE. Incubate at 37° C. for 30 minutes. During the 30 minute incubation, dilute the 20 mM tryptase substrate stock solution [(Z-Ala-Lys-Arg-AMC2TFA; Enzyme Systems Products, #AMC-246)] 1:2000 in tryptase assay buffer [0.1 M Hepes (pH 7.5), 10% w/v Glycerol, 10 uM Heparin (Sigma H-4898) 0.01% NaN₃]. Spin plates at 1000 rpm for 10 min to pellet cells. Transfer 25 ul of supernatant to a 96well black bottom plate and add 100 ul of freshly diluted tryptase substrate solution to each well. Incubate plates at room temperature for 30 min. Read the optical density of the plates at 355nm/460nm on a spectrophotometric plate reader.

Leukotriene C4 (LTC4) is also quantified using an ELISA kit on appropriately diluted supernatant samples (determined empirically for each donor cell population so that the sample measurement falls within the standard curve) following the supplier's instructions.

### Example 10

### CHMC High Cell Density IgE Activation: Degranulation (Trytase, Histamine), Leukotriene (LTC4). and Cytokine (TNFaliha, IL-13) Assays

Cultured human mast cells (CHMC) are sensitized for 5 days with IL-4 (20 ng/ml), SCF (200 ng/ml), IL-6 (200 ng/ml), and Human IgE (CP 1035K from Cortx Biochem, 100-500ng/mi depending on generation) in CM medium. After sensitizing, cells are counted, pelleted (1000 rpm, 5-10 minutes), and resuspended at 1-2×10⁶ cells/ml in MT buffer. Add 100 ul of cell suspension to each well and 100 ul of compound dilutions. The final vehicle concentration is 0.5% DMSO. Incubate at 37□C. (5% CO₂) for 1 hour. After 1 hour of compound treatment, stimulate cells with 6X anti-IgE. Mix wells with the cells and allow plates to incubate at 37□C. (5% CO₂) for one hour. After 1 hour incubation, pellet cells (10 minutes, 1000 RPM) and collect 200 ul per well of the supernatant, being careful not to disturb pellet. Place the supernatant plate on ice. During the 7-hour step (see next) perform tryptase assay on supernatant that had been diluted 1:500. Resuspend cell pellet in 240 ul of CM media containing 0.5% DMSO and corresponding concentration of compound. Incubate CHMC cells for 7 82 hours at 37□C. (5% CO₂). After incubation, pellet cells (1000 RPM, 10 minutes) and collect 225 ul per well and place in −80□C. until ready to perform ELISAS. ELISAS are performed on appropriately diluted samples (determined empirically for each donor cell population so that the sample measurement falls within the standard curve) following the supplier's instructions.

### Example 11

### BMMC High Cell Density IgE Activation: Degranulation (Hexosiminidase. Histamine), Leukotriene (LTC4), and Cytokine (TNFalpha. IL-6) Assays Preparation of WEHI-Conditioned Medium

WEHI-conditioned medium is obtained by growing murine myelomonocytic WEHI-3B cells (American Type Culture Collection, Rockville, Md.) in Iscove's Modified Eagles Media (Mediatech, Hernandon, VA) supplemented with 10% heat-inactivated fetal bovine serum (FBS; JRH Biosciences, Kansas City, Mo.), 50 □M 2-mercaptoethanol (Sigma, St. Louis, Mo.) and 100 IU/mL penicillin-steptomycin (Mediatech) in a humidified 37□C., 5% CO₂/95% air incubator. An initial cell suspension is seeded at 200,000 cells/mL and then split 1:4 every 3-4 days over a period of two weeks. Cell-free supernatants are harvested, aliquoted and stored at −80□C. until needed.

Preparation of BMMC Medium

BMMC media consists of 20% WEHI-conditioned media, 10% heat-inactivated FBS (JHR Biosciences), 25 mM HEPES, pH7.4 (Sigma), 2 mM L-glutamine (Mediatech), 0.1 mM non-essential amino acids (Mediatech), 1 mM sodium pyruvate (Mediatech), 50 □M 2-mercaptoethanol (Sigma) and 100 IU/mL penicillin-streptomycin (Mediatech) in RPMI 1640 media (Mediatech). To prepare the BMMC Media, all components are added to a sterile IL filter unit and filtered through a 0.2 □m filter prior to use.

Protocol

Bone marrow derived mast cells (BMMC) are sensitized overnight with murine SCF (20 ng/ml) and monoclonal anti-DNP (10 ng/ml, Clone SPE-7, Sigma # D-8406) in BMMC media at a cell density of 666×10³ cells/ml. After sensitizing, cells are counted, pelleted (1000 rpm, 5-10 minutes), and resuspended at 1-3×10⁶ cells/ml in MT buffer. Add 100 ul of cell suspension to each well and 100 ul of compound dilutions. The final vehicle concentration is 0.5% DMSO. Incubate at 37□C. (5% CO₂) for 1 hour. After 1 hour of compound treatment, stimulate cells with 6× stimulus (60 ng/ml DNP-BSA). Mix wells with the cells and allow plates to incubate at 37□C. (5% CO₂) for one hour. After 1 hour incubation, pellet cells (10 minutes, 1000 RPM) and collect 200 ul per well of the supernatant, being careful not to disturb pellet, and transfer to a clean tube or 96-well plate. Place the supernatant plate on ice. During the 4-5 hour step (see next) perform the hexosiminidase assay. Resuspend cell pellet in 240 ul WEI-conditioned media containing 0.5% DMSO and corresponding concentration of compound. Incubate BMMC cells for 4-5 hours at 37□C. (5% CO₂). After incubation, pellet cells (1000 RPM, 10 minutes) and collect 225 ul per well and place in −80□C. until ready to perform ELISAS. ELISAS are performed on appropriately diluted samples (determined empirically for each donor cell population so that the sample measurement falls within the standard curve) following the supplier's instructions.

Hexosaminidase assay: In a solid black 96-well assay plate, add 50 uL hexosaminidase substrate (4methylumbelliferyl-N-acetyl-□-D-glucosaminide; 2 mM) to each well. Add 50 uL of BMMC cell supernatant (see above) to the hexoseaminidase substrate, place at 37□C. for 30 minutes and read the plate at 5, 10, 15, and 30 minutes on a spectrophotometer.

### Example 12

### Basophil IgE or Dustmite Activation: Histamine Release Assay (watch tense)

The basophil activation assay is carried out using whole human peripheral blood from donors allergic to dust mites with the majority of the red blood cells removed by dextran sedimentation. Human peripheral blood is mixed 1:1 with 3% dextran T500 and RBCs are allowed to settle for 20-25min. The upper fraction is diluted with 3 volumes of D-PBS and cells are spun down for 10 min at 1500 rpm, RT. Supernatant is aspirated and cells are washed in an equal volume MT-buffer. Finally, cells are resuspended in MT-buffer containing 0.5% DMSO in the original blood volume. 80 uL cells are mixed with 20 uL compound in the presence of 0.5% DMSO, in triplicate, in a V-bottom 96-well tissue culture plate. A dose range of 8 compound concentrations is tested resulting in a 10-point dose response curve including maximum (stimulated) and minimum (unstimulated) response. Cells are incubated with compound for 1 hour at 37□C., 5% CO₂ after which 20 uL of 6× stimulus [1 ug/mL anti-IgE (Bethyl Laboratories) 667 au/mL house dustmite (Antigen Laboratories)] is added. The cells are stimulated for 30 minutes at 37□C., 5% CO₂. The plate is spun for 10 min at 1500 rpm at room temperature and 80 uL the supernatant is harvested for histamine content analysis using the histamine ELISA kit supplied by Immunotech. The ELISA is performed according to supplier's instructions.

### Example 13

### Monocyte Activation

This protocol measures cell surface markers of monocyte activation THP-1, U937 monocyte cell lines transfected with siRNA (see previous protocols) or infected with retroviral. Transfected or infected cells grown at 37□C. in 5% CO₂ are stimulated with IFNy for either 3 days (U937) or 4 days (THP-1) cells in the appropriate growth media. The cells are treated with Nozyme to release them from the plate, then stained with various antibodies against CD11 b, CD32, CD14, CD64, and HLA-DR conjugated to FITC, phycoerythrin (PE) or allophytin conugate (APC). As a control naive cells were stained and compared to stimulated cells.

### Example 14

### Osteoclast Differentiation Assay

This protocol is used to measure osteoclast differentiation in osteoclast precursors expressing a dominant negative mutant or siRNA. Differentiation is induced by treatment with TRANCE and MCSF.

Mouse cells: From bone marrow, spleen, or the monocytic cell line RAW264.7:

Mouse bone marrow cells or spleen cells are cultured in α-MEM (Life Technologies, Grand Island, N.Y.) containing 10% FBS with M-CSF (5 ng/mi) for 12 h in 100-mm diameter dishes (Corning, Glass, Corning, N.Y.; 1×10⁷ cells/10 ml/dish) to separate adherent cells and nonadherent cells. Then, nonadherent cells are harvested and cultured with M-CSF (30 ng/ml) in 100-mm diameter dishes (1×10⁷ cells/10 ml/dish). After 2 days of culture, floating cells are removed and attached cells are used as osteoclast precursors. To generate osteociasts, osteoclast precursors are cultured with TRANCE (300 ng/ml) and M-CSF (30 ng/ml) for 3 days in 96-well culture plates (Corning; 2×10⁴ cells/0.2 ml/well) or in 60-mm diameter dishes (Corning; 2.5×10⁶ cells/5 ml/dish). To purify mature osteoclasts, cells are treated with cell dissociation solution (Sigma-Aldrich) for 5 min, and the sides of the plates are tapped. Most mononuclear cells are detached after tapping, but multinucleated osteoclasts remained attached to the culture plates. To generate osteoclasts from the murine myeloid RAW264.7 cell line (American Type Culture Collection, Manassas, VA), cells are cultured in 96-well culture plates (1×10³ cells/0.2 ml/well) with TRANCE (300 ng/ml) for 4 days. Old media are replaced with fresh media containing TRANCE (300 ng/ml) on day 3. To generate human osteociasts, freshly isolated human peripheral blood monocytes are cultured in 96-well culture plates (5×10⁴ cells/0.2 ml/well) with TRANCE (300 ng/ml) and M-CSF (30 ng/ml) for 5 days. Old media are replaced with fresh media containing TRANCE (300 ng/ml) and M-CSF (30 ng/ml) on day 3. In some experiments, indicated concentration of PGN, poly(l:C) RNA, LPS, or CpG DNA is added to the cultures with or without TRANCE and MCSF. All cells are cultured at 37□C. and 5% CO₂.

Osteoclast formation is measured by a tartrate-resistant acid phosphatase (TRAP) solution assay or TRAP staining as described (Mol Cell. 1999 Dec;4(6):1041-9, Nature. 2002 Jul 25;418(6896):443-7).

For human cells: THP-1cells, human PBMC, human CD14+PBMC, U937 cells, human bone marrow. Osteolcast differentiation is induced by treating the cells in the appropriate media with recombinant soluble TRANCE (10-100 ng/mL) and M-CSF (10-100 ng/mL) as described (Calcif Tissue Int. 1998 Jun;62(6):527-31).. Fresh media and cytokines are added every 3-4 days. Typically multinucleated giant cells are produced in 5 days B 3 weeks. Osteoclast formation is measured by a tartrateresistant acid phosphatase (TRAP) solution assay or TRAP staining as described (Mol Cell. 1999 Dec;4(6):1041-9, Nature. 2002 Jul 25;418(6896):443-7).

### Example 15

### Hcs Pad Assay

Following staining, as described below, the cells are analyzed using the methods described in U.S. Ser. No. ______ (attorney docket no. RIGL-016-

00US), filed Aug. 28, 2002.

Fix and Dapi stain Procedure

Using Hudson Plate Crane, Bio-Tek Elx405 plate washer, and Labsystems Multidrop 384

Plates should be Packard View black 96-well plates #6005182, clear plate seals #6005185 PBS B calcium & magnesium-free Cellgro cat # 21-040-CM Supplies: plate seals, marker, 20 uL pipettman & tips, 5 mL tube and holder, conical 5 mL flasks & holder, timer, 1 mg/mL DAPI stock

Make fix and warm

Fix is 7.4% formaldehyde in PBS MUST BE PRE-WARMED TO 37° C.

| | Add mL of 10% | To mL warm PBS, then |
|---|---|---|
| Number of plates | formaldehyde stock | place in incubator to warm |
| 1 plate | 7.4 mL | 2.6 mL |
| 12 (round up to 15) | 111 | 39 |
| 24 (round up to 30) | 222 | 78 |

HCS_FIX method on robot removes media down to 100 uL, then adds 100 uL of fix, FAC: 3.7% formaldehyde. Make fix for 10 mL per plate, plus 6 mL dead vol. in Multidrop tubing.

Make Dapi int. stock in DW

| | | | Then add this mixture |
|---|---|---|---|
| | | | to mL PBS |
| Number of | Add uL of | To mL | just before use, |
| plates | 1 mg/mL DAPI stock | DW | shake immediately |
| 12 plates | 18 uL | 7.2 mL | 300 mL |

HCS_DAPI method removes wash down to 30 uL, then adds 170 uL of DAPI per well, FAC′ 0.5 ng/mL DAPI in PBS. Make DAPI for 17 mL per plate, plus 6 mL dead volumn

Empty robot's waste bottle and rinse

Put fresh PBS in correct bottle, transfer drawing tube and prime the system full of PBS

SET MULTIDROP TO 100 uL, 96 well plate and 12 columns and PRIME the Multidrop with formaldehyde

Take plates out of incubator and stack with flange facing inward, label w/ bar code

RUN HCS_FIX and 5_TO_{—}4, START TIMER COUNTDOWN FROM 30 MIN when fix goes on the first plate

At 30 minute mark, if have 12 plates, set methods for:

HCS_WASH 5_TO_4
HCS_DAPI
5_TO_4 (if less than 12, stop here & time
15 minutes from DAPI onto first plate)
HCS_WASH
5_TO_4
HCS_WASH

As the wash begins, CHANGE MULTIDROP TO 170 uL, rinse tubing and PRIME with DAPI

CLEANUP

Seal plates and store in frig

Empty waste bottle and rinse

Transfer drawing tube to water bottle and prime the system full of water

Clean and remove Multidrop tubing and place in drawer, reset Multidrop to 100 uL

Fixative:

Polysciences, Inc. Cat#04018, 1 liter, 10% formaldehyde (methanol-free) ultrapure EM grade

DAPI:

Molecular Probes D-1306 10 mg

Dilute to 5 mg/mL in DW, keep in frig. Make 1 mg/mL stock in DW and store in fig for 3 months

### Example 16

### Dissociated Spinal Cord Cultures

Primary cultures of dissociated spinal cord and DRGs are prepared as described by Roy et al. (1998). In brief, spinal cords and associated ganglia are dissected from embryos, dissociated with trypsin, and plated on 12-mm coverslips precoated with poly-D-lysine and extracellular matrix (Sigma-Aldrich) at a density of 2.5×10⁵ cells per well of a four-well plate (Nunclon). Approximately 1 B2×106 cells are obtained from each spinal cord, each cord being processed and plated separately. For microinjection studies, cultures are prepared from embryos and plated at a density of 6.5×10⁵ per well in 12-well dishes (Roy et al., 1998). All cells are plated in modified N3 medium as described in Roy et al. (1998). On days 4 and 5, cultures are treated with 1 μM cytosine arabinoside for 1 B2 d to limit growth of nonneuronal cells, and are maintained in modified N3 medium at 37□C. in 5% CO². Cultures are used for analyses after 14 d in vitro studies and after 4B6 wk for microinjection studies.

### Example 17

### DRG NeuronBdissociated Spinal Cord Cocultures

DRG cultures are prepared as described in O'Ferrall et al. (2000) with the following modifications. The medium for plating and general maintenance is as for the dissociated spinal cord cultures described above. DRG neurons are plated at 12B15 dissociated DRGs per well of a four-well plate containing coverslips precoated as above.

For coculture experiments, Falcon cell culture inserts (0.4 μM polyethylene terephthalate track etched membrane, six-well format; Becton Dickinson) are placed in six-well insert companion plates that contained medium only, or that had been preplated with dissociated spinal cord cultures at a density of 106 cells per well. DRG neurons are plated on glass coverslips as described above and allowed to establish for 4 d. Coverslips are then transferred to Falcon cell culture inserts and cocultured with the dissociated spinal cord cultures or with medium only for 10B14 d. After this time, coverslips are removed and labeled using the TUNEL assay as a marker of apoptosis.

Immunocvtochemistrv

Immunocytochemistry is performed as in Roy et al. (1998) using antibodies from Chemicon (peripherin, monoclonal MAB1527, and polyclonal AB1515; poylclonal neurofilament antibodies to NF-L, AB1983; NF-M, AB1981; and neurofilament heavy subunit [NF-H], AB1982; all 1:1,000), SigmaAldrich (monoclonal antibodies to neurofilaments NF-L, NR4; NF-M, NN18; NF-H, N52; and -tubulin, DM1A; all 1:1,000), and nuclear envelope breakdown (polyclonal antibody to activated caspase-3, 1:100; following supplier recommendations). Antibody distribution is visualized by epifluorescence/confocal microscopy after incubation with the appropriate secondary antibody (Alexa FluorBlabeled secondary antibody; 1:100; Molecular Probes).

For electron microscopy and immunohistochemical analysis of transgenic mouse tissue sections, the method of Beaulieu et al. (1999) is used. Immunoblotting Cells are harvested in 7 mM Tris, pH 6.75, containing 2% SDS and 10% glycerol, and assayed for total protein using the bicinchoninic acid assay. Loadings of 10B15 μg of protein are routinely analyzed on 6B12% gradient SDSBpolyacrylamide gels and then blotted to polyvinyldifluoride membrane. For immunoblotting, membranes are incubated with monoclonal antibodies recognizing peripherin (MAB1527,1:5,000; Chemicon) or actin (MAB1501,1:10,000; Chemicon), and antibody binding is revealed using the ECL detection system (NEN Life Sciences).

TUNEL Assavs

The In Situ Cell Death Detection Kit, POD, from Roche Molecular Diagnostics (Laval, QC) is used for TUNEL assays, with DAB as the substrate (Gavrieli et al., 1992). Fluorescent double labeling of cultures with antibody to peripherin is performed in conjunction with the TUNEL assay to enable correlation of TUNEL-positive cells with the presence of peripherin aggregates. TUNEL labeling in itself is not indicative of apoptosis, and confirmatory evidence of apoptosis is obtained from morphological criteria such as cell shrinkage and maintenance of an intact plasma membrane, chromatin condensation, clearly observed with DAB-TUNEL labeling and labeling with antibody recognizing activated caspase-3 (Wyllie, 1980; Majno and Joris, 1995; Thornberry and Lazebnik, 1998; Nijhawan et al., 2000). TUNEL-positive DRG neurons from dissociated spinal cord cultures are counted after 14 and 21 d in culture. To calculate the percentage of TUNEL-positive DRG neurons, cell cultures are counted using the 25x objective covering ten fields in the vertical axis and ten in the horizontal axis. Individual cultures are counted a minimum of three times and each time no less than 100 DRG neurons are counted. The percentage specific apoptosis (% experimental apoptosis−% spontaneous apoptosis/100−% spontaneous apoptosis) is calculated using the averages of the total counts from Per and WT cultures from the same litter. This enables a direct comparison between different culturing experiments.

### Example 18

### Cell Cycle analysis with BrdU

Cells ( A549, Hela) are plated 24 hours before transfection on 24-well plate ( Costar) in 500 ?I growth media supplemented with 10% FBS.

siRNA are obtained from Dharmacon Inc. or Xeragon. Inc.

60 pmol of siRNA duplex is mixed with 50 μl of Opti-Mem media (Gibco). In another tube 3 μl of Oligofectamine Reagent (Invitrogen) is mixed with 12 μl of Opti-Mem media and incubated 10 min at room temperature. Solutions are combined and incubated 25 min at room temperature. Then 32 μl of fresh of Opti-Mem media is added to final volume of 100 μl . The 100 μl of siRNA- Oligofectamine mix is added to the cells. 16 hours after transfection cells are ished 2 times with PBS, trypsinized and plated on 6 well plate with density 2500 cells/cm² for Cell Cycle analysis with BrdU and FACScan instrument or 1500 cells per well onto 96 well tissue citure plate (Costar) for PAD assay with Cellomics instrument.

72 hours after transfection BrdU is added at concentration 10 μ.

4 hours after incubation with BrdU cells are collected, fixed and prepared for Cell Cycle analysis as previously described (Kastan et al.,1991, Cancer research, 51:6304-6311; White etal.,1994, Genes and Development 8: 666-677; Serrano et al, 1997, Cell, 88(5):593-602, which are expressly incorporated herein by reference). Cell cycle analysis is performed using a Becton Dickinson FACScan instrument.

### Example 19

### GFP Cell Tracker

Cell tracker assays are performed as is known in the art and described in the Molecular Probes catalog. Cells also are contacted with or co-express negative effectors deubiquitinating agents.

We claim:

1) A method of assaying for a candidate modulating agent that modulates the cleavage of a ubiquitin complex by a deubiquitinating agent, said method comprising the steps of:
a) combining:
i) a candidate modulating agent;
ii) a ubiquitin complex;
iii) a deubiquitinating agent; and
b) assaying for the modulation of said cleavage by said candidate modulating agent.

2) The method according to claim 1, wherein said candidate agent is an organic molecule.

3) The method according to claim 1, wherein said deubiquitinating agent is a protein comprising an amino acid sequence selected from the group consisting of the amino acid sequences represented by the amino acid sequence accession numbers presented in Table 1 or encoded by the nucleic acid sequences represented by the nucleic acid sequence accession numbers presented in Table 1 or an allelic variant thereof, or a functional fragment thereof.

4) The method according to claim 1, wherein said ubiquitin complex comprises a target protein comprising at least one ubiquitin moiety.

5) The method of claim 4, wherein said ubiquitin moiety is selected from the group consisting of ubiquitin, NEDD8, ISG-15, APG12, APG8, Fat10, Fau, SUMO-1, SUMO-2 and SUMO-3.

6) The method according to claim 4, wherein said target protein comprises a first FRET label and said ubiquitin moiety comprises a second FRET label.

7) The method according to claim 4, wherein one member of said ubiquitin complex comprises a FRET label and another member of said ubiquitin complex comprises a Quencher.

8) The method according to claim 4, wherein one member of said ubiquitin complex comprises an attachment moiety.

9) The method according to claim 4, wherein said member of said ubiquitin complex comprising an attachment moiety is attaches to a solid support.

10) The method according to claim 4, wherein said member of said ubiquitin complex comprising an attachment moiety is attaches to a microtiter plate.

11) The method according to claim 4, wherein said member of said ubiquitin complex comprising an attachment moiety is attached to a bead.

22) The method according to claim 1, wherein said ubiquitin complex comprises a ubiquitin agent comprising at least one ubiquitin moiety, wherein said ubiquitin agent is bound to said at least one ubiquitin via an isopeptide bond or a peptide bond.

33) The method according to claim 12, wherein said ubiquitin agent is selected from the group consisting of a ubiquitin ligating agent and a ubiquitin conjugating agent.

44) The method according to claim 13, wherein said ubiquitin agent comprises a first FRET label and said ubiquitin moiety comprises a second FRET label.

55) The method according to claim 13, wherein said one member of said ubiquitin complex comprises a FRET label and another member of said ubiquitin complex comprises a Quencher.

16) The method according to claim 13, wherein one member of said ubiquitin complex comprises an attachment moiety.

17) The method according to claim 13, wherein said member of said ubiquitin complex comprising an attachment moiety is provided on a solid support.

18) The method according to claim 17, wherein said member of said ubiquitin complex comprising an attachment moiety is provided on a microtiter plate.

19) The method according to claim 17, wherein said member of said ubiquitin complex comprising an attachment moiety is attached to a bead.

20) The method according to claim 1, wherein said ubiquitin complex comprises a cleavable ubiquitin fusion polypeptide.

21) The method according to claim 20, wherein said cleavable ubiquitin fusion polypeptide comprises a first ubiquitin moiety bound, via a peptide bond or an isopeptide bond, to a second ubiquitin moiety.

22) The method of claim 20, wherein said cleavable ubiquitin fusion polypeptide comprises a ubiquitin moiety bound, via a peptide bond or an isopeptide bond, to a polypeptide.

23) The method according to claim 20, wherein said cleavable ubiquitin fusion polypeptide is a branched ubiquitin peptide comprising a first branch and a second branch,
a) said first branch comprising, from amino to carboxyl terminus:
i) flanking amino acids 1, 2, and 3
ii) a branched lysine, K; and
iii) flanking amino acids 4, 5, and 6,
wherein said flanking amino acids 1, 2, 3, 4, 5, and 6 are selected from amino acids flanking the lysine in a ubiquitin substrate and located within about 10-20 amino acids from said lysine in said ubiquitin substrate; and
b) said second branch comprising an amino acid sequence encoded by the
C-terminus of a ubiquitin moiety,
wherein said amino acid sequence is at least about 3-20 amino acids in length, and wherein said second branch is joined to said branched lysine of said first branch.

24) The method according to claim 23, wherein said amino acid sequence of said second branch is, from amino to carboxyl terminus, LRLRGG.

25) The method according to claim 24, wherein said first branch comprises the amino acid sequence, from amino to carboxyl terminus, KSSTYKTVA.

26) The method according to claim 20, wherein said cleavable ubiquitin fusion polypeptide comprises at least one tag.

27) The method according to claim 26, wherein said cleavable ubiquitin fusion polypeptide comprises a first tag and a second tag.

28) The method according to claim 27, wherein said first tag is on one side of the cleavable bond and said second tag is on the other side of the cleavable.bond of said cleavable ubiquitin fusion polypeptide.

29) The method according to claim 28, wherein said first tag is a first label and said second tag is a second label.

30) The method according to claim 29, wherein said first label is a first FRET label and said second label is a second FRET label.

31) The method according to claim 29, wherein first label is a FRET label and said second label is a Quencher of said FRET label.

32) The method according to claim 28, wherein said first tag comprises a Flag tag and said second tag comprises a His tag.

33) The method according to claim 28, wherein said cleavable ubiquitin fusion polypeptide comprises a first ubiquitin moiety comprising said first tag bound, via a peptide bond or an isopeptide bond, to a second ubiquitin moiety comprising said second tag.

34) The method according to claim 33, wherein said first tag is at the amino terminus of said first ubiquitin moiety and said second tag is at the carboxyl terminus of said second ubiquitin moiety.

35) The method according to claim 34, wherein only one of said first tag or said second tag is selected from the group consisting of a FLAG tag, a His tag and a GST tag.

36) The method according to claim 35, wherein the other of said first tag or said second tag is label.

37) A method of assaying for a candidate modulating agent that modulates the cleavage of a ubiquitin complex in a cell by a deubiquitinating agent, said method comprising the steps of:
a) providing a cell comprising a deubiquitinating agent and a ubiquitin complex; b) introducing into said cell a candidate modulating agent; and
c) assaying for the modulation of said cleavage by said candidate modulating agent.

38) The method according to claim 37, wherein said ubiquitin complex comprises a target protein comprising at least one ubiquitin moiety.

39) The method according to claim 37, wherein said ubiquitin complex comprises a ubiquitin agent comprising at least one ubiquitin moiety.

40) The method according to claim 39, wherein said ubiquitin agent is a ubiquitin ligating agent or a ubiquitin conjugating agent.

41) The method according to claim 37, wherein said ubiquitin complex is a cleavable ubiquitin fusion polypeptide.

42) The method according to claim 37, wherein said cell is a mammalian cell.

43) A method comprising:
a) contacting a cell with a negative effector of a deubiquitinating agent;
b) screening said cell for an altered phenotype, whereby said ubiquitin agent is identified as a modulator of said phenotype.

44) The method of claim 43, wherein said deubiquitinating agent is a protein comprising an amino acid sequence selected from the group consisting of the amino acid sequences represented by the amino acid sequence accession numbers presented in Table 1 or encoded by the nucleic acid sequences represented by the nucleic acid sequence accession numbers presented in Table 1 or an allelic variant thereof.

45) The method of claim 43, wherein said contacting comprises introducing a nucleic acid into said cell.

46) The method of claim 45, wherein said nucleic acid is said negative effector of said deubiquitinating agent.

47) The method of claim 46, wherein said nucleic acid is an siRNA targeted against mRNA encoding said deubiquitinating agent.

48) The method of claim 46, wherein said nucleic acid is antisense to an mRNA or gene encoding said deubiquitinating agent.

49) The method of claim 45, wherein said nucleic acid comprises a sequence encoding said negative effector of said deubiquitinating agent, operably linked to transcriptional and translational regulatory elements.

50) The method of claim 49, wherein said expression construct is contained within a vector.

51) The method of claim 50, wherein said vector is a retroviral vector.

52) The method of claim 49, wherein said negative effector is selected from the group consisting of an siRNA targeted against mRNA encoding said deubiquitinating agent, nucleic acid antisense to an mRNA or gene encoding said deubiquitinating agent, and a dominant negative variant of said deubiquitinating agent.

53) The method of claim 43, wherein said altered phenotype is altered cell cycle regulation.

54) The method of claim 43, wherein said altered phenotype is altered cellular proliferation and/or altered cell viability.

55) The method of claim 43, wherein said altered phenotype is altered response to an inflammatory cytokine.

56) The method of claim 43, wherein said cell is a T cell and said altered phenotype is altered response to a T cell activating agent.

57) The method of claim 43, wherein said cell is a B cell and said altered phenotype is altered response to a B cell activating agent.

58) The method of claim 43, wherein said cell is an endothelial cell and said altered phenotype is altered response to an angiogenesis stimulating agent.

59) The method of claim 43, wherein said altered phenotype is altered chemotaxis and/or haplotaxis.

60) The method of claim 43, wherein said cell is a mast cell and said altered phenotype is altered response to mast cell activation.

61) The method of claim 43, wherein said altered phenotype is altered exocytosis.

62) The method of claim 43, wherein said altered phenotype is altered release or synthesis of LDL.

63) The method of claim 43, wherein said altered phenotype is altered response to a signaling agent.
